(19) United States
(12) Patent Application Publication
(10) Pub. No.: US 2015/0274777 A1
(43) Pub. Date: Oct. 1, 2015

(54) KETOAMIDE IMMUNOPROTEASOME INHIBITORS

(71) Applicant: HOFFMANN-LA ROCHE INC., Nutley, NJ (US)

(72) Inventors: Stephen M. Lynch, Westfield, NJ (US); Arjun Narayanan, Cambridge, MA (US); Sandra Steiner, Little Falls, NJ (US)

(21) Appl. No.: 14/434,453

(22) PCT Filed: Sep. 30, 2013
(86) PCT No.: PCT/EP2013/070351

§ 371 Date: Apr. 9, 2015

Related U.S. Application Data
(60) Provisional application No. 61/712,312, filed on Oct. 11, 2012.

(51) Int. Cl. C07K 7/02; C07K 5/02

(52) U.S. Cl. CPC C07K 7/02; C07K 5/02; A61K 38/00

(57) ABSTRACT

The invention is concerned with the compounds of formula (I): and pharmaceutically acceptable salts thereof, wherein X, R¹, R^{1′}, R², R^{2′}, R³, R⁴, R^{4′} and R⁵ are defined in the detailed description and claims. In addition, the present invention relates to methods of manufacturing and using the compounds of formula (I) as well as pharmaceutical compositions containing such compounds. The compounds of formula (I) are LMP7 inhibitors and may be useful in treating associated inflammatory diseases and disorders such as, for example, rheumatoid arthritis, lupus and irritable bowel disease.

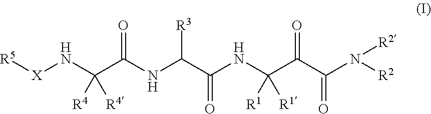

### FIELD OF THE INVENTION

The present invention relates to organic compounds useful for therapy and/or prophylaxis in a mammal of an inflammatory disease or disorder, and in particular to ketoamide compounds for the treatment of rheumatoid arthritis, lupus and irritable bowel disease (IBD), their manufacture, pharmaceutical compositions containing them and their use as LMP7 inhibitors.

### BACKGROUND OF THE INVENTION

LMP7 is an essential component of the immunoproteasome, mainly expressed in immune cells such as T/B lymphocytes and monocytes, as well as non-immune cells that have exposed to inflammatory cytokines, including IFN-γ and TNFα. The immunoproteasome plays an essential role in generation of antigenic peptide repertoire and shaping MHC class I restricted CD8+ T cell response. Moebius J. et al. European Journal of Immunology. 2010; Basler, M et al. Journal of Immunology. 2004. 3925-34. Emerging data suggested that LMP7 also regulates inflammatory cytokine production and immune cell functions beyond the regulation of MHC class I mediated antigen presentation.

A small molecule LMP7 inhibitor, PR-957, was shown to potently block Th1/17 differentiation, B cell effector functions and production of inflammatory cytokines (IL-6, TNF-α, IL-23). Muchamuel T et al. Natural Medicine. 2009. 15, 781-787; Basler M. et al. Journal of Immunology. 2010, 634-41.

In addition, LMP7 blockade with PR-957 was shown to produce therapeutic benefits in several preclinical autoimmune disease models. First, PR-957 significantly decreased disease score in mouse CAIA and CIA arthritis models, with hallmarks of significantly reduced inflammation and bone erosion. Muchamuel T. et al. Natural Medicine. 2009. 15, 781-787. In addition, PR-957 reduced plasma cells numbers and levels of anti-dsDNA IgG in MRL/lpr lupus-prone mice model, and prevented disease progression in these mice. Ichikawa H T, et al. Arthritis & Rheumatism. 2012. 64, 493-503. Furthermore, PR-957 reduced inflammation and tissue destruction in a DSS-induced colitis model in mice. Basler M. et al. Journal of Immunology. 2010, 634-41. Lastly, LMP7 knockout mice were shown to be protected from disease in IBD models. Schmidt N. et al. Gut 2010. 896-906.

Taken together, data strongly suggests that LMP7 activity is closely related to the functions of B/T lymphocytes and production of inflammatory cytokines, all of which are clinically validated targets/pathways in the pathogenesis of rheumatoid arthritis, lupus and IBD. Thus, existing data provide a strong rationale for targeting LMP7 for autoimmune disease indications. Due to potential liability with long term usage of a covalent inhibitor in chronic diseases like autoimmunity, a covalent reversible or non-covalent small molecule LMP7 inhibitor is highly desired for autoimmune disease indications.

### SUMMARY OF THE INVENTION

The invention provides for a compound of formula (I):

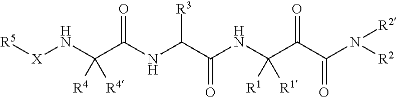

wherein:
X is —C(O)— or —S(O)₂—;
one of R¹ or R^{1′} is H or unsubstituted C₁₋₇ alkyl and the other is unsubstituted C₁₋₇ alkyl or C₁₋₇ alkyl substituted with phenyl, or
   R¹ and R^{1′}, together with the carbon atom to which they are attached, combine to form an indanyl moiety;
one of R² or R^{2′} is H or methyl and the other is cycloalkyl, unsubstituted C₁₋₇ alkyl, or C₁₋₇ alkyl substituted with phenyl, alkoxy or heteroaryl;
R³ is unsubstituted C₁₋₇ alkyl or C₁₋₇ alkyl substituted with phenyl, methoxyphenyl, indolyl, alkoxy, —SO₂CH₃, heteroaryl, chlorophenyl, heterocycle or CF₃;
one of R⁴ or R^{4′} is H or unsubstituted C₁₋₇ alkyl and the other is unsubstituted C₁₋₇ alkyl or C₁₋₇ alkyl substituted with alkoxy or cycloalkyl, or
   R⁴ or R^{4′}, together with the carbon atom to which they are attached, combine to form a cycloalkyl moiety;
R⁵ is selected from:
   CH₃C(O)NHCH(CH₂-phenylmethyl),
   isoindolyl,
   dihydroisoindolyl,
   —CH₂-heterocycle,
   —CH₂-heteroaryl,
   —CH₂—CH₂-methylpyrazolyl,
   methyl-indenyl,
   —CH₂-phenyl,
   indanyl,
   methyl-isoxazolyl,
   unsubstituted heteroaryl,
   heteroaryl mono- or bi-substituted independently with C₁₋₇ alkyl or CF₃,
   unsubstituted phenyl,
   phenyl mono- or bi-substituted independently with C₁₋₇ alkyl or halogen,
   —CH₂-benzo[1,4]oxazinyl,
   —CH₂-dihydrobenzo[1,4]oxazinyl,
   —O—CH₂-phenyl,
   methyl-indolyl,
   methyl-pyrrolo[3,2-b]pyridinyl or
   imidazo[1,2-a]pyridinyl,
or a pharmaceutically acceptable salt thereof.

The invention also provides for pharmaceutical compositions comprising the compounds, methods of using the compounds and methods of preparing the compounds.

All documents cited to or relied upon are expressly incorporated herein by reference.

### DETAILED DESCRIPTION OF THE INVENTION

Unless otherwise indicated, the following specific terms and phrases used in the description and claims are defined as follows:

The term “moiety” refers to an atom or group of chemically bonded atoms that is attached to another atom or molecule by one or more chemical bonds thereby forming part of a molecule. For example, the variables R1 to R6 of formula I refer to moieties that are attached to the core structure of formula I by a covalent bond.

In reference to a particular moiety with one or more hydrogen atoms, the term “substituted” refers to the fact that at least one of the hydrogen atoms of that moiety is replaced by another substituent or moiety. For example, the term “C₁₋₇ alkyl substituted by halogen” refers to the fact that one or more hydrogen atoms of a C₁₋₇ alkyl (as defined below) is replaced by one or more halogen atoms (e.g., trifluoromethyl, difluoromethyl, fluoromethyl, chloromethyl, etc.).

The term “alkyl” refers to an aliphatic straight-chain or branched-chain saturated hydrocarbon moiety having 1 to 20 carbon atoms. In particular embodiments the alkyl has 1 to 10 carbon atoms.

The term “C₁₋₇ alkyl” refers to an alkyl moiety having 1 to 7 carbon atoms. Examples of lower alkyls include methyl, ethyl, propyl, isopropyl, butyl, isobutyl, sec-butyl and tert-butyl.

The term “alkoxy” denotes a group of the formula —O—R′, wherein R′ is an alkyl group. Examples of alkoxy moieties include methoxy, ethoxy, isopropoxy, and tert-butoxy.

“Aryl” means a monovalent cyclic aromatic hydrocarbon moiety having a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof, each being optionally substituted.

The term “heteroaryl” denotes a monovalent aromatic heterocyclic mono- or bicyclic ring system of 5 to 12 ring atoms, comprising 1, 2, 3 or 4 heteroatoms selected from N, O and S, the remaining ring atoms being carbon. Examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, or quinoxalinyl.

The terms “halo”, “halogen” and “halide”, which may be used interchangeably, refer to a substituent fluoro, chloro, bromo, or iodo.

The term “haloalkyl” denotes an alkyl group wherein at least one of the hydrogen atoms of the alkyl group has been replaced by same or different halogen atoms, particularly fluoro atoms. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, or trifluoromethyl.

“Cycloalkyl” means a monovalent saturated carbocyclic moiety having mono- or bicyclic rings. The cycloalkyl moiety can optionally be substituted with one or more substituents. Examples of cycloalkyl moieties include, but are not limited to, cyclopropyl, cyclobutyl, cyclopentyl, cyclohexyl, cycloheptyl, and the like, including partially unsaturated (cycloalkenyl) derivatives thereof.

Unless otherwise indicated, the term “hydrogen” or “hydro” refers to the moiety of a hydrogen atom (—H) and not H₂.

Unless otherwise indicated, the term “a compound of the formula” or “a compound of formula” or “compounds of the formula” or “compounds of formula” refers to any compound selected from the genus of compounds as defined by the formula (including any pharmaceutically acceptable salt or ester of any such compound if not otherwise noted).

The term “pharmaceutically acceptable salts” refers to those salts which retain the biological effectiveness and properties of the free bases or free acids, which are not biologically or otherwise undesirable. Salts may be formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxalic acid, maleic acid, malonic acid, salicylic acid, succinic acid, fumaric acid, tartaric acid, citric acid, benzoic acid, cinnamic acid, mandelic acid, methanesulfonic acid, ethanesulfonic acid, p-toluenesulfonic acid, N-acetylcystein and the like. In addition, salts may be prepared by the addition of an inorganic base or an organic base to the free acid. Salts derived from an inorganic base include, but are not limited to, the sodium, potassium, lithium, ammonium, calcium, and magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like.

The compounds of the present invention can be present in the form of pharmaceutically acceptable salts. The compounds of the present invention can also be present in the form of pharmaceutically acceptable esters (i.e., the methyl and ethyl esters of the acids of formula I to be used as prodrugs). The compounds of the present invention can also be solvated, i.e. hydrated. The solvation can be effected in the course of the manufacturing process or can take place i.e. as a consequence of hygroscopic properties of an initially anhydrous compound of formula I (hydration).

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “a therapeutically effective amount” of a compound means an amount of compound that is effective to prevent, alleviate or ameliorate symptoms of disease or prolong the survival of the subject being treated. Determination of a therapeutically effective amount is within the skill in the art. The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 0.1 mg to about 5,000 mg, 1 mg to about 1,000 mg, or 1 mg to 100 mg may be appropriate, although the lower and upper limits may be exceeded when indicated. The daily dosage can be administered as a single dose or in divided doses, or for parenteral administration, it may be given as continuous infusion.

The term “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

In the practice of the method of the present invention, an effective amount of any one of the compounds of this invention or a combination of any of the compounds of this invention or a pharmaceutically acceptable salt or ester thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

In detail, the present invention provides for compounds of formula (I):

A compound of formula (I):

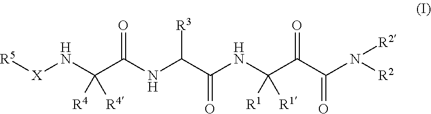

wherein:
X is —C(O)— or —S(O)₂—;
one of R¹ or R^{1′} is H or unsubstituted C₁₋₇ alkyl and the other is unsubstituted C₁₋₇ alkyl or C₁₋₇ alkyl substituted with phenyl, or
   R¹ and R^{1′}, together with the carbon atom to which they are attached, combine to form an indanyl moiety;
one of R² or R^{2′} is H or methyl and the other is cycloalkyl, unsubstituted C₁₋₇ alkyl, or C₁₋₇ alkyl substituted with phenyl, alkoxy or heteroaryl;
R³ is unsubstituted C₁₋₇ alkyl or C₁₋₇ alkyl substituted with phenyl, methoxyphenyl, indolyl, alkoxy, —SO₂CH₃, heteroaryl, chlorophenyl, heterocycle or CF₃;
one of R⁴ or R^{4′} is H or unsubstituted C₁₋₇ alkyl and the other is unsubstituted C₁₋₇ alkyl or C₁₋₇ alkyl substituted with alkoxy or cycloalkyl, or
   R⁴ or R^{4′}, together with the carbon atom to which they are attached, combine to form a cycloalkyl moiety;
R⁵ is selected from:
   CH₃C(O)NHCH(CH₂-phenylmethyl),
   isoindolyl,
   dihydroisoindolyl,
   —CH₂-heterocycle,
   —CH₂-heteroaryl,
   —CH₂—CH₂-methylpyrazolyl,
   methyl-indenyl,
   —CH₂-phenyl,
   indanyl,
   methyl-isoxazolyl,
   unsubstituted heteroaryl,
   heteroaryl mono- or bi-substituted independently with C₁₋₇ alkyl or CF₃,
   unsubstituted phenyl,
   phenyl mono- or bi-substituted independently with C₁₋₇ alkyl or halogen,
   —CH₂-benzo[1,4]oxazinyl,
   —CH₂-dihydrobenzo[1,4]oxazinyl,
   —O—CH₂-phenyl,
   methyl-indolyl,
   methyl-pyrrolo[3,2-b]pyridinyl or
   imidazo[1,2-a]pyridinyl,
or a pharmaceutically acceptable salt thereof.

In another embodiment of the invention, provided is a compound according to formula (I), wherein X is —C(O)—.

In another embodiment of the invention, provided is a compound according to formula (I), wherein one of R¹ or R^{1′} is H and the other is butyl or —CH₂-phenyl.

In another embodiment of the invention, provided is a compound according to formula (I), wherein one of R¹ or R^{1′} is H and the other is —CH₂-phenyl.

In another embodiment of the invention, provided is a compound according to formula (I), wherein one of R² or R^{2′} is H and the other is cyclopropyl, methyl, —CH₂-phenyl, —CH₂—CH₂-phenyl, —CH₂CH₂OCH3 or —CH₂-pyridinyl.

In another embodiment of the invention, provided is a compound according to formula (I), wherein R² or R^{2′} is H and the other is —CH₂-phenyl.

In another embodiment of the invention, provided is a compound according to formula (I), wherein R³ is tert-butyl, iso-butyl, —CH₂-phenyl, —CH₂-methoxyphenyl, —CH₂-indolyl, —CH₂-methoxy, —CH₂CH₂SO₂CH₃, —CH₂-pyranyl, —CH₂-pyridinyl, —CH₂-chlorophenyl, —CH₂-tetrahydropyranyl or —CH₂CF₃.

In another embodiment of the invention, provided is a compound according to formula (I), wherein R³ is —CH₂-methoxyphenyl or —CH₂-indolyl.

In another embodiment of the invention, provided is a compound according to formula (I), wherein one of R⁴ or R^{4′} is H and the other is methyl, tert-butyl, —CH₂—OCH₃ or cyclopropyl.

In another embodiment of the invention, provided is a compound according to formula (I), wherein R⁴ or R^{4′}, together with the carbon atom to which they are attached, combine to form a cyclopropyl moiety.

In another embodiment of the invention, provided is a compound according to formula (I), wherein one of R⁴ or R^{4′} is H and the other is methyl.

In another embodiment of the invention, provided is a compound according to formula (I), wherein R⁵ is CH₃C(O)NHCH(CH₂-phenylmethyl), -dihydroindolyl, —CH₂-morpholine, —CH₂—CH₂-methylpyrazolyl, methyl-indenyl, —CH₂-phenyl, indanyl, methyl-isoxazolyl, pyrazinyl, methyl-pyrazolyl, dimethyl-pyrazolyl, ethyl-pyrazolyl, methyl-trifluoromethyl-pyrazolyl, phenyl, dichloro-phenyl, methyl-phenyl, —CH₂-benzo[1,4]oxazinyl, —CH₂-dihydrobenzo[1,4]oxazinyl, —O—CH₂-phenyl, methyl-indolyl, methyl-pyrrolo[3,2-b]pyridinyl or imidazo[1,2-a]pyridinyl.

In another embodiment of the invention, provided is a compound according to formula (I), wherein R⁵ is indanyl or —O—CH₂-phenyl.

Another embodiment of the invention relates to a compound of formula (I′):

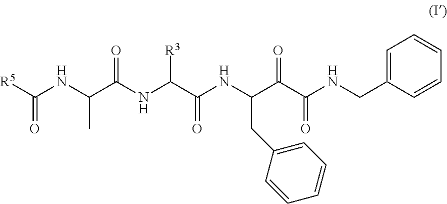

wherein R³ and R⁵ are as defined above, or a pharmaceutically acceptable salt thereof.

A particular embodiment of the invention relates to a compound of formula (I′) wherein

R³ is —CH₂-methoxyphenyl or —CH₂-indolyl,
R⁵ is indanyl or —O—CH₂-phenyl,
or a pharmaceutically acceptable salt thereof.

In another embodiment of the invention, provided are compounds of formula (I) wherein the compound is:
(S)-3-{(S)-2-[(S)-2-((S)-2-Acetylamino-3-o-tolyl-propionylamino)-3,3-dimethyl-butyrylamino]-4-methyl-pentanoylamino}-2-oxo-heptanoic acid benzylamide;
(S)-3-{(S)-2-[(S)-3,3-Dimethyl-2-(2-morpholin-4-yl-acetylamino)-butyrylamino]-4-methyl-pentanoylamino}-2-oxo-heptanoic acid benzylamide;
(S)-3-{(S)-3-(4-Methoxy-phenyl)-2-[(S)-2-(2-morpholin-4-yl-acetylamino)-propionylamino]-propionylamino}-2-oxo-heptanoic acid benzylamide;
5-Methyl-isoxazole-3-carboxylic acid {(S)-1-[(S)-1-((S)-1-benzylaminooxalyl-pentylcarbamoyl)-2-(1H-indol-3-yl)-ethylcarbamoyl]-ethyl}-amide;
3-Methyl-1H-indene-2-carboxylic acid {(S)-1-[(S)-1-((S)-1-benzylaminooxalyl-pentylcarbamoyl)-2-(1H-indol-3-yl)-ethylcarbamoyl]-ethyl}-amide;
5-Methyl-isoxazole-3-carboxylic acid {(S)-1-[(S)-1-((S)-1-benzylaminooxalyl-pentylcarbamoyl)-2-methoxy-ethylcarbamoyl]-ethyl}-amide;
3-Methyl-1H-indene-2-carboxylic acid ((S)-1-{(S)-2-(1H-indol-3-yl)-1-[(S)-1-((S)-1-phenyl-ethylaminooxalyl)-pentylcarbamoyl]-ethylcarbamoyl}-ethyl)-amide;
3-Methyl-1H-indene-2-carboxylic acid ((S)-1-{(S)-2-(1H-indol-3-yl)-1-[(S)-1-((R)-1-phenyl-ethylaminooxalyl)-pentylcarbamoyl]-ethylcarbamoyl}-ethyl)-amide;
{(S)-1-[(S)-1-((S)-1-Benzylaminooxalyl-pentylcarbamoyl)-2-(1H-indol-3-yl)-ethylcarbamoyl]-ethyl}-carbamic acid benzyl ester;
(S)-3-{(S)-3-(1H-Indol-3-yl)-2-[(S)-2-(2-morpholin-4-yl-acetylamino)-propionylamino]-propionylamino}-2-oxo-heptanoic acid benzylamide;
Indan-2-carboxylic acid {(S)-1-[(S)-1-((S)-1-benzylaminooxalyl-pentylcarbamoyl)-2-(1H-indol-3-yl)-ethylcarbamoyl]-ethyl}-amide;
N-{(S)-1-[(S)-1-((S)-1-Benzylaminooxalyl-pentylcarbamoyl)-2-(1H-indol-3-yl)-ethylcarbamoyl]-ethyl}-2,3-dichloro-benzamide;
2-Methyl-2H-pyrazole-3-carboxylic acid {(S)-1-[(S)-1-((S)-1-benzylaminooxalyl-pentylcarbamoyl)-2-(1H-indol-3-yl)-ethylcarbamoyl]-ethyl}-amide;
(S)-3-{(S)-3-(4-Methoxy-phenyl)-2-[(S)-2-(2-morpholin-4-yl-acetylamino)-propionylamino]-propionylamino}-2-oxo-heptanoic acid methylamide;
3-Methyl-1H-indene-2-carboxylic acid {(S)-1-[(S)-1-((S)-1-benzylaminooxalyl-pentylcarbamoyl)-3-methanesulfonyl-propylcarbamoyl]-ethyl}-amide;
{(S)-1-[(S)-1-((S)-1-Benzyl-2-benzylcarbamoyl-2-oxo-ethylcarbamoyl)-2-(1H-indol-3-yl)-ethylcarbamoyl]-ethyl}-carbamic acid benzyl ester;
{(S)-1-[(S)-1-((S)-1-Benzyl-2-benzylcarbamoyl-2-oxo-ethylcarbamoyl)-2-(4-methoxy-phenyl)-ethylcarbamoyl]-ethyl}-carbamic acid benzyl ester;
Indan-2-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide;
Indan-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
3-Methyl-1H-indene-2-carboxylic acid {(S)-1-[(S)-1-((S)-1-benzyl-2-benzylcarbamoyl-2-oxo-ethylcarbamoyl)-3-methanesulfonyl-propylcarbamoyl]-ethyl}-amide;
5-Methyl-isoxazole-3-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
(S)—N-Benzyl-3-{(S)-3-(1H-indol-3-yl)-2-[(S)-2-(2-morpholin-4-yl-acetylamino)-propionylamino]-propionylamino}-2-oxo-4-phenyl-butyramide;
Indan-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-2-methoxy-ethyl}-amide;
Indan-2-carboxylic acid {(S)-1-[(S)-1-((S)-1-benzyl-2-benzylcarbamoyl-2-oxo-ethylcarbamoyl)-3-methyl-butylcarbamoyl]-ethyl}-amide;
3-Methyl-1H-indene-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
Indan-2-carboxylic acid {(S)-[(S)-1-((S)-1-benzyl-2-benzylcarbamoyl-2-oxo-ethylcarbamoyl)-2-(4-methoxy-phenyl)-ethylcarbamoyl]-cyclopropyl-methyl}-amide;
Indan-2-carboxylic acid {1-[(S)-1-((S)-1-benzyl-2-benzylcarbamoyl-2-oxo-ethylcarbamoyl)-2-(4-methoxy-phenyl)-ethylcarbamoyl]-cyclopropyl}-amide;
{1-[(S)-1-((S)-1-Benzyl-2-benzylcarbamoyl-2-oxo-ethylcarbamoyl)-2-(4-methoxy-phenyl)-ethylcarbamoyl]-cyclopropyl}-carbamic acid benzyl ester;
Pyrazine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
2-Methyl-2H-pyrazole-3-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide;
{(S)-1-[(S)-1-((S)-1-Benzyl-2-methylcarbamoyl-2-oxo-ethylcarbamoyl)-2-(1H-indol-3-yl)-ethylcarbamoyl]-ethyl}-carbamic acid benzyl ester;
{(S)-1-[(S)-1-((S)-1-Benzyl-2-cyclopropylcarbamoyl-2-oxo-ethylcarbamoyl)-2-(1H-indol-3-yl)-ethylcarbamoyl]-ethyl}-carbamic acid benzyl ester;
{(S)-1-[(S)-1-[(S)-1-Benzyl-2-(2-methoxy-ethylcarbamoyl)-2-oxo-ethylcarbamoyl]-2-(1H-indol-3-yl)-ethylcarbamoyl]-ethyl}-carbamic acid benzyl ester;
{(S)-1-[(S)-1-{(S)-1-Benzyl-2-oxo-2-[(pyridin-2-ylmethyl)-carbamoyl]-ethylcarbamoyl}-2-(1H-indol-3-yl)-ethylcarbamoyl]-ethyl}-carbamic acid benzyl ester;
(S)—N-Benzyl-3-[(S)-2-[(S)-2-(2-2,3-dihydro-benzo[1,4]oxazin-4-yl-acetylamino)-propionylamino]-3-(4-methoxy-phenyl)-propionylamino]-2-oxo-4-phenyl-butyramide;
{(S)-1-[(S)-1-((S)-1-Benzyl-2-benzylcarbamoyl-2-oxo-ethylcarbamoyl)-3-methyl-butylcarbamoyl]-ethyl}-carbamic acid benzyl ester;
2-Methyl-2H-pyrazole-3-carboxylic acid {(S)-1-[(S)-1-((S)-1-benzyl-2-benzylcarbamoyl-2-oxo-ethylcarbamoyl)-3-methyl-butylcarbamoyl]-ethyl}-amide;
{(S)-1-[(S)-1-[(S)-1-Benzyl-2-(benzyl-methyl-carbamoyl)-2-oxo-ethylcarbamoyl]-2-(1H-indol-3-yl)-ethylcarbamoyl]-ethyl}-carbamic acid benzyl ester;
(S)-3-[(S)-2-((S)-2-Benzenesulfonylamino-propionylamino)-3-(4-methoxy-phenyl)-propionylamino]-N-benzyl-2-oxo-4-phenyl-butyramide;
(S)—N-Benzyl-3-{(S)-3-(4-methoxy-phenyl)-2-[(S)-2-(toluene-2-sulfonylamino)-propionylamino]-propionylamino}-2-oxo-4-phenyl-butyramide;
(S)—N-Benzyl-3-((S)-3-(4-methoxy-phenyl)-2-{(S)-2-[3-(2-methyl-2H-pyrazol-3-yl)-propionylamino]-propionylamino}-propionylamino)-2-oxo-4-phenyl-butyramide;
Indan-2-carboxylic acid {(S)-1-[(S)-1-((S)-1-benzyl-2-benzylcarbamoyl-2-oxo-ethylcarbamoyl)-3-phenyl-propylcarbamoyl]-ethyl}-amide;
Indan-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
1-Methyl-1H-indole-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
1-Methyl-1H-pyrrolo[3,2-b]pyridine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
(S)—N-Benzyl-3-{(S)-3-(4-methoxy-phenyl)-2-[(S)-2-(2-morpholin-4-yl-acetylamino)-propionylamino]-propionylamino}-2-oxo-4-phenyl-butyramide;
5-Methyl-isoxazole-3-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide;
3-Methyl-1H-indene-2-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide;
Imidazo[1,2-a]pyridine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
{(S)-1-[(S)-1-((S)-1-Benzyl-2-benzylcarbamoyl-2-oxo-ethylcarbamoyl)-2-pyridin-2-yl-ethylcarbamoyl]-ethyl}-carbamic acid benzyl ester;
{(S)-1-[(S)-1-((S)-1-Benzyl-2-benzylcarbamoyl-2-oxo-ethylcarbamoyl)-2-(4-chloro-phenyl)-ethylcarbamoyl]-ethyl}-carbamic acid benzyl ester;
1,3-Dihydro-isoindole-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
2,5-Dimethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
2-Ethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
2-Methyl-5-trifluoromethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
{(S)-1-[1-((S)-1-Benzyl-2-benzylcarbamoyl-2-oxo-ethylcarbamoyl)-2-(tetrahydro-pyran-4-yl)-ethylcarbamoyl]-ethyl}-carbamic acid benzyl ester;
{(S)-1-[1-((S)-1-Benzyl-2-benzylcarbamoyl-2-oxo-ethylcarbamoyl)-3,3,3-trifluoro-propylcarbamoyl]-ethyl}-carbamic acid benzyl ester;
{(S)-1-[(S)-1-((S)-1-Benzyl-2-benzylcarbamoyl-2-oxo-ethylcarbamoyl)-2-pyridin-4-yl-ethylcarbamoyl]-ethyl}-carbamic acid benzyl ester; or
Indan-2-carboxylic acid {(S)-1-[(S)-1-(2-benzylaminooxalyl-indan-2-ylcarbamoyl)-2-(4-methoxy-phenyl)-ethylcarbamoyl]-ethyl}-amide; or
   pharmaceutically acceptable salts thereof.

In another embodiment of the invention, provided are compounds of formula (I) wherein the compound is:
{(S)-1-[(S)-1-((S)-1-Benzyl-2-benzylcarbamoyl-2-oxo-ethylcarbamoyl)-2-(1H-indol-3-yl)-ethylcarbamoyl]-ethyl}-carbamic acid benzyl ester;
{(S)-1-[(S)-1-((S)-1-Benzyl-2-benzylcarbamoyl-2-oxo-ethylcarbamoyl)-2-(4-methoxy-phenyl)-ethylcarbamoyl]-ethyl}-carbamic acid benzyl ester;
Indan-2-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide; or
Indan-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide; or
   pharmaceutically acceptable salts thereof.

In another embodiment, the invention provides for a pharmaceutical composition, comprising a therapeutically effective amount of a compound according to formula (I) and a pharmaceutically acceptable carrier.

In another embodiment, the invention provides for a compound according to formula (I) for use as a therapeutically active substance.

In another embodiment, the invention provides for the use of a compound according to formula (I) for the treatment or prophylaxis of an inflammatory disease or disorder.

In another embodiment, the invention provides for the use of a compound according to formula (I) for the preparation of a medicament for the treatment or prophylaxis of an inflammatory disease or disorder.

In another embodiment, the invention provides for a compound according to formula (I) for the treatment or prophylaxis of an inflammatory disease or disorder.

In another embodiment, the invention provides for a method for treating an inflammatory disease or disorder selected from rheumatoid arthritis, lupus and irritable bowel disease (IBD), comprising the step of administering a therapeutically effective amount of a compound according to formula (I) to a subject in need thereof.

In another embodiment, provided is an invention as hereinbefore described.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40.

The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Compounds of the invention may be made by any number of conventional means. For example, they may be made according to the processes outlined in Schemes 1 to 3 below.

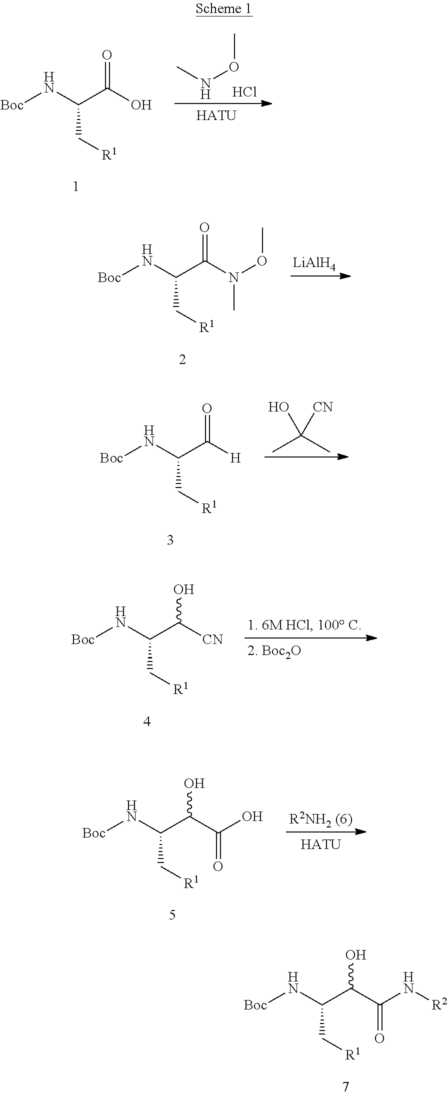

As seen in Scheme 1, the N-Boc protected amino acid 1 can be converted to the Weinreb amide 2 then can be reduced to the aldehyde 3 using lithium aluminum hydride (LiAlH₄). The aldehyde can be immediately treated with acetone cyanohydrin to form the new cyanohydrin 4 as a mixture of diastereomers. The nitrile can be hydrolyzed to the carboxylic acid by heating with hydrochloric acid along with loss of the Boc protecting group. The Boc group can be reinstalled using di-tert-butyl dicarbonate to afford the acid 5 which can be subsequently coupled with an appropriate amine 6 using an activating reagent such as HATU to provide the hydroxyamide 7.

The R groups can be moieties as described in, for example, the Examples and claims.

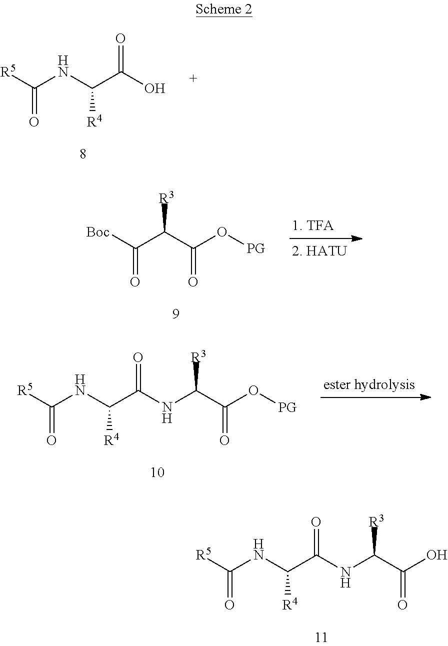

As shown in Scheme 2, an N-Boc amino acid 9 bearing appropriate carboxyl protection can be treated with trifluoroacetic acid (TFA). The free amine salt thus generated can be coupled in situ with an appropriately functionalized amino acid 8 using an activating reagent such as HATU to afford the dipeptidyl ester 10. Acid 11 can result from ester hydrolysis under variable conditions.

The R groups can be moieties as described in, for example, the Examples and claims.

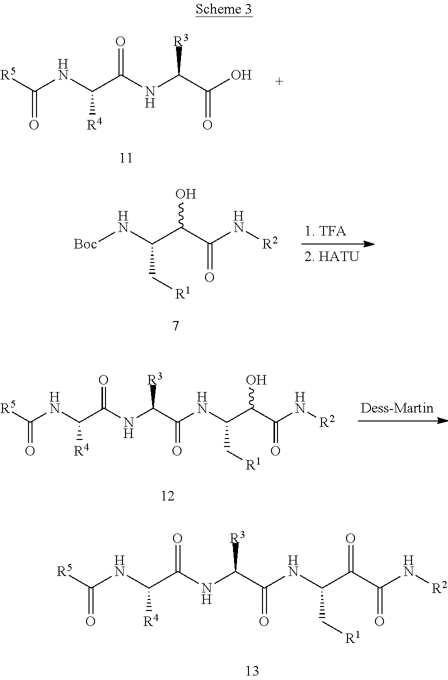

According to scheme 3, the hydroxyamide 7 can be treated with trifluoroacetic acid (TFA). The free amine salt thus generated can be coupled in situ with acid 11 using an activating reagent such as HATU to afford hydroxyamide 12. Ketoamide 13 can be provided by oxidation with Dess-Martin periodinane. The R groups can be moieties as described in, for example, the Examples and claims.

### EXAMPLES

Although certain exemplary embodiments are depicted and described herein, the compounds of the present invention can be prepared using appropriate starting materials according to the methods described generally herein and/or by methods available to one of ordinary skill in the art. All reactions involving air-sensitive reagents were performed under an inert atmosphere. Reagents were used as received from commercial suppliers unless otherwise noted.

I. Preparation of Certain Intermediates

### Intermediate 1

### [(S)-1-(Benzylcarbamoyl-hydroxy-methyl)-pentyl]-carbamic acid tert-butyl ester

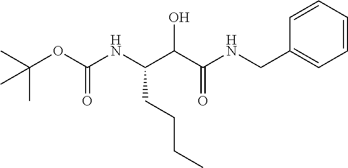 [SMILES: CCCC[C@H](NC(=O)OC(C)(C)C)C(O)C(=O)NCc1ccccc1]

To a suspension of (S)-2-(tert-butoxycarbonylamino)hexanoic acid (2.16 g, 9.34 mmol) and N,O-dimethylhydroxylamine hydrochloride (1.37 g, 14.0 mmol) in DMF (20 ml) was added HATU (3.73 g, 9.81 mmol) and N,N-diisopropylethylamine (4.9 ml, 28.0 mmol). The reaction mixture was stirred at room temperature overnight then quenched with water and extracted with EtOAc (3×). The combined organics were washed with water (3×) and brine then dried over MgSO₄ and concentrated to afford 2.64 g of [(S)-1-(methoxy-methyl-carbamoyl)-pentyl]-carbamic acid tert-butyl ester as a viscous colorless oil which was used without further purification.

To a solution of [(S)-1-(methoxy-methyl-carbamoyl)-pentyl]-carbamic acid tert-butyl ester (2.56 g, 9.34 mmol) in THF (30 ml) at 0° C. was slowly added LiAlH₄ (1.0 M in THF, 9.34 ml, 9.34 mmol). The reaction mixture was stirred at 0° C. for 45 min then carefully quenched with solid sodium sulfate decahydrate. When gas evolution had ceased, EtOAc was added and the reaction mixture was stirred vigorously at room temperature for 30 min. The mixture was filtered over Buchner funnel, rinsing with EtOAc. The filtrate was concentrated to afford 2.24 g of ((S)-1-formyl-pentyl)-carbamic acid tert-butyl ester as a colorless oil which was used immediately in the next step without further purification.

To a solution of ((S)-1-formyl-pentyl)-carbamic acid tert-butyl ester (2.01 g, 9.34 mmol) in dichloromethane (20 ml) were added acetone cyanohydrin (2.56 ml, 28.0 mmol) and triethylamine (0.78 ml, 5.6 mmol). The reaction was stirred at room temperature for 4 h then diluted with Et₂O (100 ml) and washed with water (5×). The organic phase was dried over MgSO₄ and concentrated. The residue was purified by silica gel chromatography (10% to 35% EtOAc/hexanes) to afford 1.85 g (82%, 3 steps) of [(S)-1-(cyano-hydroxy-methyl)-pentyl]-carbamic acid tert-butyl ester as a pale yellow oil.

To [(S)-1-(cyano-hydroxy-methyl)-pentyl]-carbamic acid tert-butyl ester (1.85 g, 7.63 mmol) was added aqueous 6 M HCl (30 ml). The reaction mixture was heated at 100° C. for 15 h then cooled to room temperature and concentrated under reduced pressure. The sticky solid residue was dissolved in 1,4-dioxane (15 ml) and water (15 ml) then sodium bicarbonate (6.41 g, 76.3 mmol) and di-tert-butyl dicarbonate (2.5 g, 11.5 mmol) were added. The heterogeneous reaction mixture was stirred vigorously at room temperature overnight. The organic phase was removed under reduced pressure. The remaining heterogeneous aqueous layer was diluted with water and extracted with Et₂O (discarded). Then the aqueous layer was brought to pH=3 by addition of aqueous 2 M HCl. The aqueous layer was extracted with Et₂O and EtOAc. These extracts were combined, dried over MgSO₄ and concentrated to provide 1.14 g (57%) of (S)-3-tert-butoxycarbonylamino-2-hydroxy-heptanoic acid as a viscous colorless oil which was used without further purification.

To a solution of (S)-3-tert-butoxycarbonylamino-2-hydroxy-heptanoic acid (540 mg, 2.07 mmol) in DMF (8 ml) were added benzylamine (0.27 ml, 2.48 mmol), HATU (864 mg, 2.27 mmol), and N,N-diisopropylethylamine (0.54 ml, 3.1 mmol). The bright yellow reaction mixture was stirred at room temperature overnight then quenched with water and extracted with EtOAc (2×). The combined organics were washed with water (3×) and brine then dried over MgSO₄ and concentrated. The residue was purified by silica gel chromatography (20% to 50% EtOAc/hexanes) to isolate 464 mg (64%) of [(S)-1-(benzylcarbamoyl-hydroxy-methyl)-pentyl]-carbamic acid tert-butyl ester as a white solid. LC/MS: (M-Boc)⁺=251.

### Intermediate 2

### ((S)-1-Benzyl-2-benzylcarbamoyl-2-hydroxy-ethyl)-carbamic acid tert-butyl ester

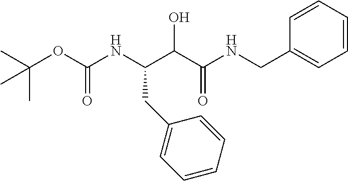 [SMILES: CC(C)(C)OC(=O)N[C@@H](Cc1ccccc1)C(O)C(=O)NCc1ccccc1]

To a solution of (S)-2-tert-butoxycarbonylamino-3-phenyl-propionic acid (25 g, 94.34 mmol) in DMF (250 mL) were added N,O-dimethylhydroxylamine hydrochloride (13.72 g, 141.50 mmol), HATU (37.64 g, 99.05 mmol) and N,N-diisopropylethylamine (50.70 mL, 283.01 mmol) under nitrogen atmosphere at room temperature. The reaction mixture was stirred at room temperature for 16 h then diluted with ethyl acetate (1000 mL) and washed with water (5×250 mL). The organic layer was dried and concentrated under reduced pressure. The crude residue was purified by CombiFlash column chromatography using 20% EtOAc in hexane to afford 27.5 g (94%) of (S)-1-(methoxy-methyl-carbamoyl)-2-phenyl-ethyl]-carbamic acid tert-butyl ester as colorless oil. LC/MS: (M+H)⁺=309.0.

To a stirred solution of (S)-1-(methoxy-methyl-carbamoyl)-2-phenyl-ethyl]-carbamic acid tert-butyl ester (15 g, 48.70 mmol) in THF (180 mL) at 0° C. was added LiAlH₄ (1.0 M in THF, 57 mL, 57 mmol). The reaction mixture was stirred at 0° C. for 1 h then carefully quenched by portionwise addition of sodium sulfate decahydrate until gas evolution ceased. EtOAc was added and the reaction mixture was stirred vigorously at room temperature for 30 min and then filtered. The filtrate was dried and concentrated under reduced pressure to afford 11.0 g (91%) of ((S)-1-benzyl-2-oxo-ethyl)-carbamic acid tert-butyl ester as white solid which was used without further purification.

To a solution of ((S)-1-benzyl-2-oxo-ethyl)-carbamic acid tert-butyl ester (7.0 g, 28.1 mmol) in DCM (80 mL) was added acetone cyanohydrin (7.16 g, 84.3 mmol) and triethylamine (2.36 mL, 16.86 mmol). The reaction was stirred at room temperature for 3 h then water was added and the organics were removed under reduced pressure. The aqueous residue was extracted with ethyl acetate and washed twice with water. The organic layer was dried and concentrated under reduced pressure. The crude residue was purified by CombiFlash column chromatography using 20% EtOAc in hexane as mobile phase to obtain 5.0 g (58%) of ((S)-1-benzyl-2-cyano-2-hydroxy-ethyl)-carbamic acid tert-butyl ester as yellow oil. LC/MS: (M+H)⁺=277.4.

A solution of ((S)-1-benzyl-2-cyano-2-hydroxy-ethyl)-carbamic acid tert-butyl ester (5.0 g, 18.11 mmol) in 6M HCl (90 mL) was heated at 100° C. for 16 h then cooled to room temperature and concentrated under vacuum to afford 4.0 g (95%) of (S)-3-amino-2-hydroxy-4-phenyl-butyric acid hydrochloride as off yellow solid which was used without further purification. LC/MS: (M+H)⁺=196.2.

To a solution of (S)-3-amino-2-hydroxy-4-phenyl-butyric acid hydrochloride (18.0 g, 77.9 mmol) in 1,4-dioxane (150 ml) and water (150 mL) were added sodium bicarbonate (65.45 g 779 mmol) and di-tert-butyl dicarbonate (25.48 g, 116.9 mmol). The mixture was stirred vigorously at room temperature for 16 h. The organic phase was removed under reduced pressure. The remaining heterogeneous aqueous layer was diluted with water (200 mL) and extracted with Et₂O (2×200 mL, discarded). Then the aqueous layer was brought to pH=3 by addition of aqueous 2 M HCl and extracted with EtOAc (3×400 mL). The combined extracts were dried and concentrated under reduced pressure to afford 18.0 g (78%) of (S)-3-tert-butoxycarbonylamino-2-hydroxy-4-phenyl-butyric acid as off white solid. LC/MS: (M+H)⁺=296.6.

To a stirred solution of (S)-3-tert-butoxycarbonylamino-2-hydroxy-4-phenyl-butyric acid (10.0 g, 33.89 mmol) in DMF (150 mL) were added benzylamine (4.35 g, 40.67 mmol), HATU (14.16 g, 37.28 mmol) and N,N-diisopropylethylamine (6.56 g, 50.84 mmol). The reaction mixture was stirred under nitrogen atmosphere at room temperature for 3 h then diluted with ethyl acetate (800 mL) and washed with ice cold water (2×950 mL). The organic layer was dried over sodium sulfate and concentrated under reduced pressure. The crude residue was purified by CombiFlash column chromatography using 30% EtOAc in hexane to provide 7.3 g (56%) of (S)-1-benzyl-2-benzylcarbamoyl-2-hydroxy-ethyl)-carbamic acid tert-butyl ester as white solid. LC/MS: (M+H)⁺=385.2.

II. Preparation of Certain Embodiments of the Invention

### Example 1

### (S)-3-{(S)-2-[(S)-2-((S)-2-Acetylamino-3-o-tolyl-propionylamino)-3,3-dimethyl-butyrylamino]-4-methyl-pentanoylamino}-2-oxo-heptanoic acid benzylamide

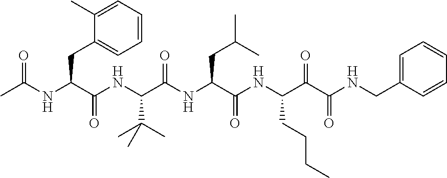 [SMILES: CCCC[C@H](NC(=O)[C@H](CC(C)C)NC(=O)[C@@H](NC(=O)[C@H](Cc1ccccc1C)NC(C)=O)C(C)(C)C)C(=O)C(=O)NCc1ccccc1]

To a solution of (S)-2-(tert-butoxycarbonylamino)-3,3-dimethylbutanoic acid (600 mg, 2.59 mmol), (S)-benzyl 2-amino-4-methylpentanoate 4-methylbenzenesulfonate (1.07 g, 2.72 mmol), and HATU (1.09 g, 2.85 mmol) in DMF (10 ml) at 0° C. was added N,N-diisopropylethylamine (1.36 ml, 7.78 mmol). The bright yellow reaction mixture was stirred at room temperature overnight then quenched with water and extracted with EtOAc (2×). The combined organics were washed with sat'd NaHCO₃, water (3×) and brine then dried over MgSO₄ and concentrated to afford 1.29 g of (S)-2-((S)-2-tert-butoxycarbonylamino-3,3-dimethyl-butyrylamino)-4-methyl-pentanoic acid benzyl ester as a white solid which was used without further purification.

To a solution of (S)-2-((S)-2-tert-butoxycarbonylamino-3,3-dimethyl-butyrylamino)-4-methyl-pentanoic acid benzyl ester (550 mg, 1.27 mmol) in CH₂Cl₂ (10 ml) was added trifluoroacetic acid (2 ml). The reaction was stirred at room temperature for 2 h then concentrated. The residue was dissolved in DMF (8 ml) and N,N-diisopropylethylamine (1.13 ml, 6.44 mmol) was added followed by (S)-2-(tert-butoxycarbonylamino)-3-o-tolylpropanoic acid (300 mg, 1.07 mmol) and HATU (449 mg, 1.18 mmol). The yellow reaction mixture was stirred at room temperature overnight then quenched with water and extracted with EtOAc (2×). The combined organics were washed with water (3×) and brine then dried over MgSO₄ and concentrated. The residue was purified by silica gel chromatography (10% to 30% EtOAc/hexanes) to afford 495 mg (77%) of (S)-2-[(S)-2-((S)-2-tert-butoxycarbonylamino-3-o-tolyl-propionylamino)-3,3-dimethyl-butyrylamino]-4-methyl-pentanoic acid benzyl ester as a white foamy solid.

To a solution of (S)-2-[(S)-2-((S)-2-tert-butoxycarbonylamino-3-o-tolyl-propionylamino)-3,3-dimethyl-butyrylamino]-4-methyl-pentanoic acid benzyl ester (490 mg, 0.82 mmol) in CH₂Cl₂ (10 ml) was added trifluoroacetic acid (2 ml). The reaction was stirred at room temperature for 2.5 h then concentrated. The residue was dissolved in CH₂Cl₂ (7 ml), cooled to 0° C. and added N,N-diisopropylethylamine (0.58 ml, 3.29 mmol) followed by acetyl chloride (64 μl, 0.91 mmol). The reaction mixture was stirred at room temperature for 1 h then quenched with water and extracted with CH₂Cl₂. The combined organics were washed with water then dried over MgSO₄ and concentrated. The residue was purified by silica gel chromatography (50% to 100% EtOAc/hexanes) to afford 344 mg (78%) of (S)-2-[(S)-2-((S)-2-acetylamino-3-o-tolyl-propionylamino)-3,3-dimethyl-butyrylamino]-4-methyl-pentanoic acid benzyl ester as an off-white solid.

To a solution of (S)-2-[(S)-2-((S)-2-acetylamino-3-o-tolyl-propionylamino)-3,3-dimethyl-butyrylamino]-4-methyl-pentanoic acid benzyl ester (345 mg, 0.64 mmol) in MeOH (15 ml) was added 10% palladium on carbon (wet) (68 mg, 0.06 mmol). The reaction mixture was stirred under a hydrogen balloon at room temperature for 1.5 h then filtered over Celite, rinsing with EtOAc/MeOH. The filtrate was concentrated then the residue was redissolved in MeOH and filtered through a 0.2 uM nylon flitted disk. The filtrate was concentrated to afford 290 mg of (S)-2-[(S)-2-((S)-2-acetylamino-3-o-tolyl-propionylamino)-3,3-dimethyl-butyrylamino]-4-methyl-pentanoic acid as a white solid.

To a solution of [(S)-1-(benzylcarbamoyl-hydroxy-methyl)-pentyl]-carbamic acid tert-butyl ester (132 mg, 0.38 mmol) in CH₂Cl₂ (3 ml) was added trifluoroacetic acid (0.5 ml). The reaction mixture was stirred at room temperature for 1.5 h then concentrated and chased with hexanes to provide a colorless oil. This oil was dissolved in DMF (2.5 ml) and N,N-diisopropylethylamine (0.25 ml, 1.45 mmol) was added followed by (S)-2-[(S)-2-((S)-2-acetylamino-3-o-tolyl-propionylamino)-3,3-dimethyl-butyrylamino]-4-methyl-pentanoic acid (130 mg, 0.29 mmol) and HATU (121 mg, 0.32 mmol). The reaction mixture was stirred at room temperature for 3 h then quenched with water. The resultant thick white precipitate was collected via filtration, rinsed with water and dried under high vacuum to afford 183 mg (93%) of (S)-3-{(S)-2-[(S)-2-((S)-2-acetylamino-3-o-tolyl-propionylamino)-3,3-dimethyl-butyrylamino]-4-methyl-pentanoylamino}-2-hydroxy-heptanoic acid benzylamide as a white solid.

To a solution of (S)-3-{(S)-2-[(S)-2-((S)-2-acetylamino-3-o-tolyl-propionylamino)-3,3-dimethyl-butyrylamino]-4-methyl-pentanoylamino}-2-hydroxy-heptanoic acid benzylamide (90 mg, 0.13 mmol) in CH₂Cl₂ (3 ml) was added Dess-Martin periodinane (62 mg, 0.15 mmol). Within a few minutes a thick white precipitate had formed. The slurry was stirred at room temperature for 45 min then sat'd NaHCO₃ (2 ml) and aqueous 10% Na₂S₂O₃ (2 ml) were added. The biphasic mixture was stirred vigorously for 15 min then the layers were separated and the aqueous phase was extracted with CH₂Cl₂ (2×). The combined organics were dried over MgSO₄ and concentrated. The residue was absorbed on silica gel and purified by chromatography (50% to 100% EtOAc/hexanes) to afford 22 mg (25%) of (S)-3-{(S)-2-[(S)-2-((S)-2-acetylamino-3-o-tolyl-propionylamino)-3,3-dimethyl-butyrylamino]-4-methyl-pentanoylamino}-2-oxo-heptanoic acid benzylamide as a white solid. LC/MS: (M+H)⁺=678.

### Example 2

### (S)-3-{(S)-2-[(S)-3,3-Dimethyl-2-(2-morpholin-4-yl-acetylamino)-butyrylamino]-4-methyl-pentanoylamino}-2-oxo-heptanoic acid benzylamide

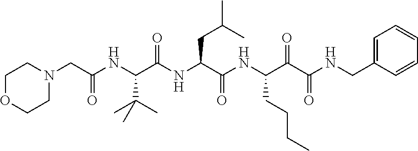 [SMILES: CCCC[C@H](NC(=O)[C@H](CC(C)C)NC(=O)[C@@H](NC(=O)CN1CCOCC1)C(C)(C)C)C(=O)C(=O)NCc1ccccc1]

To a solution of (S)-2-((S)-2-tert-butoxycarbonylamino-3,3-dimethyl-butyrylamino)-4-methyl-pentanoic acid benzyl ester (353 mg, 0.81 mmol) in dichloromethane (8 ml) was added trifluoroacetic acid (2 ml). The reaction was stirred at room temperature for 4 h then concentrated. The residue was dissolved in DMF (3 ml) and N,N-diisopropylethylamine (0.72 ml, 4.13 mmol) was added. Then added 2-morpholinoacetic acid (100 mg, 0.69 mmol) followed by HATU (288 mg, 0.76 mmol). The yellow reaction mixture was stirred at room temperature overnight then quenched with water and extracted with EtOAc (2×). The combined organics were washed with water (3×) and brine then dried over MgSO₄ and concentrated. The residue was purified by silica gel chromatography with 50% to 100% EtOAc/hexanes to afford 290 mg (91%) of (S)-2-[(S)-3,3-dimethyl-2-(2-morpholin-4-yl-acetylamino)-butyrylamino]-4-methyl-pentanoic acid benzyl ester as a white foamy solid.

To a solution of (S)-2-[(S)-3,3-dimethyl-2-(2-morpholin-4-yl-acetylamino)-butyrylamino]-4-methyl-pentanoic acid benzyl ester (290 mg, 0.63 mmol) in MeOH (12 ml) was added 20% palladium hydroxide on carbon (70 mg, 0.10 mmol). The reaction mixture was stirred under a hydrogen balloon at room temperature for 1.5 h then filtered over Celite, rinsing with EtOAc/MeOH. The filtrate was concentrated then the residue was redissolved in EtOAc/MeOH and filtered through a 0.2 uM nylon fitted disk. The filtrate was concentrated to afford 242 mg of (S)-2-[(S)-3,3-dimethyl-2-(2-morpholin-4-yl-acetylamino)-butyrylamino]-4-methyl-pentanoic acid as a white solid.

To a solution of [(S)-1-(benzylcarbamoyl-hydroxy-methyl)-pentyl]-carbamic acid tert-butyl ester (101 mg, 0.29 mmol) in CH₂Cl₂ (3 ml) was added trifluoroacetic acid (0.5 ml). The reaction mixture was stirred at room temperature for 2 h then concentrated and chased with hexanes to provide a colorless oil. This oil was dissolved in DMF (2 ml) and N,N-diisopropylethylamine (0.19 ml, 1.1 mmol) was added. Then added (S)-2-[(S)-3,3-dimethyl-2-(2-morpholin-4-yl-acetylamino)-butyrylamino]-4-methyl-pentanoic acid (82 mg, 0.22 mmol) and HATU (92 mg, 0.24 mmol). The reaction mixture was stirred at room temperature overnight then quenched with water. The resultant thick white precipitate was collected via filtration, rinsed with water and dried under high vacuum to afford 93 mg (70%) of (S)-3-{(S)-2-[(S)-3,3-dimethyl-2-(2-morpholin-4-yl-acetylamino)-butyrylamino]-4-methyl-pentanoylamino}-2-hydroxy-heptanoic acid benzylamide as a white solid and mixture of epimers. LC/MS: (M+H)⁺=604.

To a solution of (S)-3-{(S)-2-[(S)-3,3-dimethyl-2-(2-morpholin-4-yl-acetylamino)-butyrylamino]-4-methyl-pentanoylamino}-2-hydroxy-heptanoic acid benzylamide (90 mg, 0.15 mmol) in CH₂Cl₂ (3 ml) was added Dess-Martin periodinane (70 mg, 0.16 mmol). Within a few minutes a thick white precipitate had formed. The slurry was stirred at room temperature for 1 h then sat'd NaHCO₃ (2 ml) and 10% Na₂S₂O₃ (2 ml) were added. The biphasic mixture was stirred vigorously for 15 min then the layers were separated and the aqueous phase was extracted with CH₂Cl₂ (2×). The combined organics were dried over MgSO₄ and concentrated. The residue was absorbed on silica gel and purified by chromatography with 30% to 100% EtOAc/hexanes to provide 27 mg (30%) of (S)-3-{(S)-2-[(S)-3,3-dimethyl-2-(2-morpholin-4-yl-acetylamino)-butyrylamino]-4-methyl-pentanoylamino}-2-oxo-heptanoic acid benzylamide as a white solid. LC/MS: (M+H)⁺=602.

### Example 3

### (S)-3-{(S)-3-(4-Methoxy-phenyl)-2-[(S)-2-(2-morpholin-4-yl-acetylamino)-propionylamino]-propionylamino}-2-oxo-heptanoic acid benzylamide

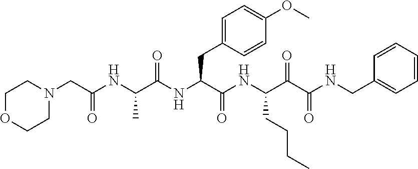 [SMILES: CCCC[C@H](NC(=O)[C@H](Cc1ccc(OC)cc1)NC(=O)[C@H](C)NC(=O)CN1CCOCC1)C(=O)C(=O)NCc1ccccc1]

In a 250 ml 3-neck round-bottomed flask, Boc-O-methyl-L-tyrosine (1.50 g, 5.08 mmol) was dissolved in 65 ml dichloromethane. The colorless solution was cooled to 0° C. Triethylamine (1.09 g, 1.5 ml, 10.8 mmol) was added at 0° C. followed by 4-dimethylaminopyridine (90 mg, 0.74 mmol). A solution of benzyl chloroformate (1.04 g, 0.87 ml, 6.09 mmol) in 10 ml dichloromethane was added dropwise. The reaction mixture was stirred at 0° C. for 3 h and at room temperature for 1 h. The reaction mixture was quenched with 20 ml saturated NaHCO₃ solution and then extracted with 20 ml dichloromethane. The organic layer was washed with 20 ml saturated NaHCO₃ solution. The aqueous layers were extracted with 50 ml dichloromethane. The organic layers were combined, dried over sodium sulfate, filtered and concentrated. The residue was chromatographed over 80 g silica gel with EtOAc/hexanes (gradient 0-20% EtOAc). All fractions containing product were combined and concentrated to afford 1.97 g (96%) (S)-2-tert-butoxycarbonylamino-3-(4-methoxy-phenyl)-propionic acid benzyl ester as a white solid.

In a 100 ml round-bottomed flask, (S)-2-tert-butoxycarbonylamino-3-(4-methoxy-phenyl)-propionic acid benzyl ester (1.95 g, 4.81 mmol) was dissolved in 15 ml dichloromethane and trifluoroacetic acid (10 ml, 130 mmol) was added slowly. The reaction mixture was stirred at room temperature for 2 h then concentrated. The residue was dissolved in 14 ml DMF and the solution was stirred at 0° C. for 5 min. N,N-Diisopropylethylamine (5.18 g, 7.0 ml, 40.1 mmol) was added dropwise at 0° C. Boc-L-alanine (761 mg, 4.02 mmol) was added followed by HATU (1.68 g, 4.42 mmol). After the addition was complete, the ice bath was removed and the reaction mixture was stirred at room temperature for 48 h. The reaction mixture was quenched with water and extracted twice with 120 ml diethyl ether. The organic layers were washed twice with 15 ml water and once with 15 ml brine. The organic layers were combined, dried over sodium sulfate, filtered and concentrated to afford (S)-2-((S)-2-tert-butoxycarbonylamino-propionylamino)-3-(4-methoxy-phenyl)-propionic acid benzyl ester as a light yellow oil which was used without further purification.

In a 100 ml round-bottomed flask, (S)-2-((S)-2-tert-butoxycarbonylamino-propionylamino)-3-(4-methoxy-phenyl)-propionic acid benzyl ester (2.42 g, 3.98 mmol; purity=75%) was dissolved in 13 ml dichloromethane. Trifluoroacetic acid (8.5 ml, 110 mmol) was added slowly. The reaction mixture was stirred at room temperature for 2.5 h then concentrated. The residue was dissolved in 12 ml DMF and the solution was stirred at 0° C. for 5 min. N,N-Diisopropylethylamine (4.44 g, 6.0 ml, 34.4 mmol) was added dropwise at 0° C. 2-Morpholinoacetic acid (502 mg, 3.46 mmol) was added followed by HATU (1.45 g, 3.8 mmol). After the addition was complete, the ice bath was removed and the reaction mixture was stirred at room temperature overnight. The reaction mixture was quenched with 15 ml of water and extracted twice with 120 ml diethyl ether. The organic layers were washed twice with 15 ml water and once with 15 ml brine. The organic layers were combined, dried over sodium sulfate, filtered and concentrated. The residue was chromatographed over 40 g silica gel with EtOAc/hexanes (gradient 0-100% EtOAc) and MeOH/EtOAc (gradient 0-5% MeOH). All fractions containing product were combined and concentrated to afford 962 mg (58%) (S)-3-(4-methoxy-phenyl)-2-[(S)-2-(2-morpholin-4-yl-acetylamino)-propionylamino]-propionic acid benzyl ester as an off-white solid.

In a 100 ml round-bottomed flask, (S)-3-(4-methoxy-phenyl)-2-[(S)-2-(2-morpholin-4-yl-acetylamino)-propionylamino]-propionic acid benzyl ester (0.96 g, 1.99 mmol) was dissolved in 20 ml methanol. The flask was alternately evacuated and flushed with argon three times. 20% Palladium hydroxide on carbon (wet, 180 mg, 0.26 mmol) was added carefully. The flask was evacuated, flushed with argon, evacuated and flushed with hydrogen. The reaction mixture was stirred under hydrogen atmosphere (balloon) at room temperature for 3 h.

The reaction mixture was filtered over Celite, rinsing with ethyl acetate/methanol. The filtrate was concentrated to afford 761 mg (97%) (S)-3-(4-methoxy-phenyl)-2-[(S)-2-(2-morpholin-4-yl-acetylamino)-propionylamino]-propionic acid as a white foam.

In a 10 ml round-bottomed flask, [(S)-1-(benzylcarbamoyl-hydroxy-methyl)-pentyl]-carbamic acid tert-butyl ester (100 mg, 0.29 mmol) was dissolved in 3 ml dichloromethane. Trifluoroacetic acid (0.5 ml, 6.49 mmol) was added slowly. The reaction mixture was stirred at room temperature for 2.5 h then concentrated. The residue was dissolved in 2 ml DMF and the solution was stirred at 0° C. for 5 min. N,N-Diisopropylethylamine (178 mg, 0.24 ml, 1.37 mmol) was added dropwise at 0° C. (S)-3-(4-Methoxy-phenyl)-2-[(S)-2-(2-morpholin-4-yl-acetylamino)-propionylamino]-propionic acid (0.090 g, 0.23 mmol) was added followed by HATU (96 mg, 0.25 mmol). After the addition was complete, the ice bath was removed and the reaction mixture was stirred at room temperature overnight. The reaction mixture was quenched with 3 ml water and extracted twice with 30 ml diethyl ether. The organic layers were washed twice with 3 ml water and once with 3 ml brine. The organic layers were combined, dried over sodium sulfate, filtered and concentrated. The combined aqueous layers were again extracted three times with 30 ml diethyl ether/EtOAc (1:1). The organic layers were combined, dried over sodium sulfate, filtered and concentrated. The combined residues were chromatographed over 12 g silica gel with MeOH/dichloromethane (gradient 0-5% MeOH). All fractions containing product were combined and concentrated to afford 130 mg (91%) (S)-2-hydroxy-3-{(S)-3-(4-methoxy-phenyl)-2-[(S)-2-(2-morpholin-4-yl-acetylamino)-propionylamino]-propionylamino}-heptanoic acid benzylamide as a white solid and as a mixture of epimers.

In a 25 ml round-bottomed flask, (S)-2-hydroxy-3-{(S)-3-(4-methoxy-phenyl)-2-[(S)-2-(2-morpholin-4-yl-acetylamino)-propionylamino]-propionylamino}-heptanoic acid benzylamide (126 mg, 0.20 mmol) was dissolved in 8 ml dichloromethane and Dess-Martin periodinane (128 mg, 0.30 mmol) was added. The reaction mixture was stirred at room temperature for 2 h. The reaction mixture was quenched with 3 ml saturated NaHCO₃ solution and 3 ml 10% Na₂S₂O₃ solution and stirred vigorously for 30 min at room temperature. The biphasic mixture was then extracted with 30 ml dichloromethane. The organic layer was washed with 5 ml saturated NaHCO₃ solution. The aqueous layers were extracted twice with 30 ml dichloromethane. The organic layers were combined, dried over sodium sulfate, filtered and concentrated. The residue was absorbed on silica gel and chromatographed over 12 g silica gel with MeOH/dichloromethane (gradient 0-5% MeOH). All fractions containing product were combined and concentrated to afford 86 mg (65%) (S)-3-{(S)-3-(4-methoxy-phenyl)-2-[(S)-2-(2-morpholin-4-yl-acetylamino)-propionylamino]-propionylamino}-2-oxo-heptanoic acid benzylamide as an off-white solid. LC/MS: (M+H)⁺=624.

### Example 4

### 5-Methyl-isoxazole-3-carboxylic acid {(S)-1-[(S)-1-((S)-1-benzylaminooxalyl-pentylcarbamoyl)-2-(1H-indol-3-yl)-ethylcarbamoyl]-ethyl}-amide

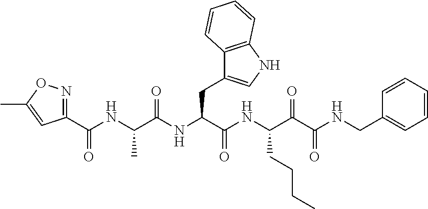 [SMILES: CCCC[C@H](NC(=O)[C@H](Cc1c[nH]c2ccccc12)NC(=O)[C@H](C)NC(=O)c1cc(C)on1)C(=O)C(=O)NCc1ccccc1]

In a 250 ml 3-neck round-bottomed flask, N-Boc-L-tryptophan (2.500 g, 8.21 mmol) was suspended in 100 ml dichloromethane. The reaction mixture was cooled to 0° C. Triethylamine (1.82 g, 2.5 ml, 17.9 mmol) was added at 0° C. followed by 4-dimethylaminopyridine (146 mg, 1.19 mmol). A solution of benzyl chloroformate (1.67 g, 1.4 ml, 9.81 mmol) in 10 ml dichloromethane was added dropwise. The reaction mixture was stirred at 0° C. for 2 h and at room temperature for 2 h. The reaction mixture was quenched with 30 ml saturated NaHCO₃-solution and then extracted with 50 ml dichloromethane. The organic layer was washed with 30 ml saturated NaHCO₃-solution. The aqueous layers were extracted with 100 ml dichloromethane. The organic layers were combined, dried over sodium sulfate, filtered and concentrated. The residue was absorbed on 10 g silica gel and chromatographed over 80 g silica gel with EtOAc/hexanes (gradient 0-30% EtOAc). All fractions containing product were combined and concentrated to afford 2.204 g (68%) (S)-2-tert-butoxycarbonylamino-3-(1H-indol-3-yl)-propionic acid benzyl ester as an off-white solid.

In a 100 ml round-bottomed flask, (S)-2-tert-butoxycarbonylamino-3-(1H-indol-3-yl)-propionic acid benzyl ester (2.200 g, 5.58 mmol) was dissolved in 18 ml dichloromethane. Trifluoroacetic acid (11 ml, 143 mmol) was added slowly. The reaction mixture was stirred at room temperature for 2 h. The solvent was evaporated and then put under high vacuum. The residue was dissolved in 18 ml DMF and the solution was stirred at 0° C. for 5 min. N,N-Diisopropylethylamine (6.66 g, 9.0 ml, 51.5 mmol) was added dropwise at 0° C. Boc-L-alanine (948 mg, 5.01 mmol) was added followed by HATU (2.1 g, 5.51 mmol). After the addition was complete, the ice bath was removed and the reaction mixture was stirred at room temperature overnight. The reaction mixture was extracted with 120 ml diethyl ether and 15 ml water. The aqueous layer was extracted with 120 ml diethyl ether. The organic layers were washed twice with 15 ml water and once with 15 ml brine. The organic layers were combined, dried over sodium sulfate, filtered and concentrated. The residue was chromatographed over 80 g silica gel with EtOAc/hexanes (gradient 0-40% EtOAc). All fractions containing product were combined and concentrated to provide 1.918 g (82%) (S)-2-((S)-2-tert-butoxycarbonylamino-propionylamino)-3-(1H-indol-3-yl)-propionic acid benzyl ester as an off-white foam.

In a 50 ml round-bottomed flask, (S)-2-((S)-2-tert-butoxycarbonylamino-propionylamino)-3-(1H-indol-3-yl)-propionic acid benzyl ester (1.915 g, 4.11 mmol) was dissolved in trifluoroacetic acid (8.0 ml, 104 mmol). The light brown solution was stirred at room temperature for 30 min. The reaction mixture was added dropwise to an ice-cold, vigorously stirred biphasic mixture of 40 ml saturated Na₂CO₃-solution and 40 ml dichloromethane and then extracted with 100 ml dichloromethane. The aqueous layer was extracted twice with 100 ml dichloromethane. The organic layers were combined, dried over sodium sulfate, filtered and concentrated to afford 1.507 g (100%) (S)-2-((S)-2-amino-propionylamino)-3-(1H-indol-3-yl)-propionic acid benzyl ester as an off-white foam which was used without further purification.

In a 50 ml round-bottomed flask, 5-methylisoxazole-3-carboxylic acid (219 mg, 1.72 mmol) and (S)-2-((S)-2-amino-propionylamino)-3-(1H-indol-3-yl)-propionic acid benzyl ester (750 mg, 2.05 mmol) were dissolved in 6.0 ml DMF. The reaction mixture was cooled to 0° C. N,N-Diisopropylethylamine (740 mg, 1.0 ml, 5.73 mmol) was added slowly at 0° C. followed by HATU (719 mg, 1.89 mmol). The yellow solution was stirred at room temperature overnight during which time the reaction mixture turned orange. The reaction mixture was extracted with 70 ml diethyl ether and 5 ml water. The aqueous layer was extracted with 70 ml diethyl ether. The organic layers were washed twice with 5 ml water and once with 5 ml brine. The organic layers were combined, dried over sodium sulfate, filtered and concentrated. The residue was chromatographed over 25 g silica gel with EtOAc/hexanes (gradient 0-70% EtOAc). All fractions containing product were combined and concentrated to afford 540 mg (66%) (S)-3-(1H-indol-3-yl)-2-{(S)-2-[(5-methyl-isoxazole-3-carbonyl)-amino]-propionylamino}-propionic acid benzyl ester as a light yellow oil.

In a 50 ml round-bottomed flask, (S)-3-(1H-indol-3-yl)-2-{(S)-2-[(5-methyl-isoxazole-3-carbonyl)-amino]-propionylamino}-propionic acid benzyl ester (530 mg, 1.12 mmol) was dissolved in 12 ml methanol. The flask was three times alternating evacuated and flushed with argon. 20% Palladium hydroxide on carbon (wet, 102 mg, 0.15 mmol) was added carefully. The flask was evacuated, flushed with argon, evacuated and flushed with hydrogen. The reaction mixture was stirred under hydrogen atmosphere (balloon) at room temperature for 3 h. The reaction mixture was filtered over Celite, rinsing with ethyl acetate/methanol. The filtrate was concentrated to afford 414 mg (87%; purity=90%) (S)-3-(1H-indol-3-yl)-2-{(S)-2-[(5-methyl-isoxazole-3-carbonyl)-amino]-propionylamino}-propionic acid as an orange foam.

In a 10 ml round-bottomed flask, [(S)-1-(benzylcarbamoyl-hydroxy-methyl)-pentyl]-carbamic acid tert-butyl ester (100 mg, 0.29 mmol) was dissolved in 3.0 ml dichloromethane. Trifluoroacetic acid (0.5 ml, 6.49 mmol) was added slowly. The reaction mixture was stirred at room temperature for 2.5 h. The solvent was evaporated and the residue placed under high vacuum for 30 min. The residue was dissolved in 2.0 ml DMF and the solution was stirred at 0° C. for 5 min. N,N-Diisopropylethylamine (185 mg, 0.25 ml, 1.43 mmol) was added dropwise at 0° C. (S)-3-(1H-Indol-3-yl)-2-{(S)-2-[(5-methyl-isoxazole-3-carbonyl)-amino]-propionylamino}-propionic acid (100 mg, 0.23 mmol; purity=90%) was added followed by HATU (98 mg, 0.26 mmol). After the addition was complete, the ice bath was removed and the reaction mixture was stirred at room temperature overnight. The reaction mixture was extracted with 40 ml diethyl ether and 3 ml water. The aqueous layer was extracted with 40 ml diethyl ether. The organic layers were washed twice with 3 ml water and once with 3 ml brine. The organic layers were combined, dried over sodium sulfate, filtered and concentrated. The residue was chromatographed over 12 g silica gel with MeOH/dichloromethane (gradient 0-5% MeOH). All fractions containing product were combined and concentrated to afford 80 mg (55%) 5-methyl-isoxazole-3-carboxylic acid {(S)-1-[(S)-1-[(S)-1-(benzylcarbamoyl-hydroxy-methyl)-pentylcarbamoyl]-2-(1H-indol-3-yl)-ethylcarbamoyl]-ethyl}-amide as an off-white solid and as a mixture of epimers.

In a 25 ml round-bottomed flask, 5-methyl-isoxazole-3-carboxylic acid {(S)-1-[(S)-1-[(S)-1-(benzylcarbamoyl-hydroxy-methyl)-pentylcarbamoyl]-2-(1H-indol-3-yl)-ethylcarbamoyl]-ethyl}-amide (73 mg, 0.12 mmol) was dissolved in 4.2 ml dichloromethane and Dess-Martin periodinane (75 mg, 0.18 mmol) was added. The reaction mixture was stirred at room temperature for 2.5 h. The reaction mixture was quenched with 2.5 ml saturated NaHCO₃-solution and 2.5 ml 10% Na₂S₂O₃-solution and stirred vigorously for 1 h at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was chromatographed over 12 g silica gel with MeOH/dichloromethane (gradient 0-5% MeOH). All fractions containing product were combined and concentrated to afford 18 mg (24%) 5-methyl-isoxazole-3-carboxylic acid {(S)-1-[(S)-1-((S)-1-benzylaminooxalyl-pentylcarbamoyl)-2-(1H-indol-3-yl)-ethylcarbamoyl]-ethyl}-amide as a light brown solid. LC/MS: (M+H)⁺=613.

### Example 5

### 3-Methyl-1H-indene-2-carboxylic acid {(S)-1-[(S)-1-((S)-1-benzylaminooxalyl-pentylcarbamoyl)-2-(1H-indol-3-yl)-ethylcarbamoyl]-ethyl}-amide

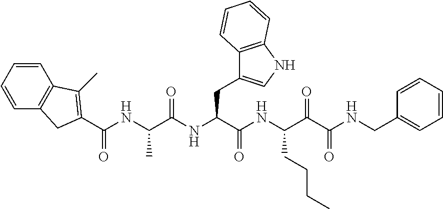 [SMILES: CCCC[C@H](NC(=O)[C@H](Cc1c[nH]c2ccccc12)NC(=O)[C@H](C)NC(=O)C1=C(C)c2ccccc2C1)C(=O)C(=O)NCc1ccccc1]

In a 100 ml round-bottomed flask, L-tryptophan tert-butyl ester hydrochloride (1.17 g, 3.94 mmol) was cooled to 0° C. 13 ml DMF was added and the solution was stirred at 0° C. for 5 min. N,N-Diisopropylethylamine (1.41 g, 1.9 ml, 10.9 mmol) was added dropwise at 0° C. N-Benzyloxycarbonyl-L-alanine (0.800 g, 3.58 mmol) was added followed by HATU (1.5 g, 3.94 mmol). After the addition was complete, the ice bath was removed and the reaction mixture was stirred at room temperature overnight. The reaction mixture was extracted with 120 ml diethyl ether and 15 ml water. The aqueous layer was extracted with 120 ml diethyl ether. The organic layers were washed twice with 15 ml water and once with 15 ml brine. The organic layers were combined, dried over sodium sulfate, filtered and concentrated to afford (S)-2-((S)-2-benzyloxycarbonylamino-propionylamino)-3-(1H-indol-3-yl)-propionic acid tert-butyl ester as an off-white foam which was used without further purification.

In a 200 ml round-bottomed flask, (S)-2-((S)-2-benzyloxycarbonylamino-propionylamino)-3-(1H-indol-3-yl)-propionic acid tert-butyl ester (1.959 g, 3.37 mmol; purity=80%) was dissolved in 35 ml methanol. The flask was three times alternating evacuated and flushed with argon. 20% Palladium hydroxide on carbon (wet, 307 mg, 0.44 mmol) was added carefully. The flask was evacuated, flushed with argon, evacuated and flushed with hydrogen. The reaction mixture was stirred under hydrogen atmosphere (balloon) at room temperature for 2.5 h. The reaction mixture was filtered over Celite, rinsing with ethyl acetate. The filtrate was concentrated to afford (S)-2-((S)-2-amino-propionylamino)-3-(1H-indol-3-yl)-propionic acid tert-butyl ester as a light yellow oil which was used without further purification.

In a 100 ml round-bottomed flask, 3-methyl-1H-indene-2-carboxylic acid (0.500 g, 2.87 mmol) and (S)-2-((S)-2-amino-propionylamino)-3-(1H-indol-3-yl)-propionic acid tert-butyl ester (1.48 g, 3.35 mmol; purity=75%) were dissolved in 10 ml DMF. The reaction mixture was cooled to 0° C. N,N-Diisopropylethylamine (1.18 g, 1.6 ml, 9.16 mmol) was added slowly at 0° C. followed by HATU (1.2 g, 3.16 mmol). The yellow solution was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then placed under high vacuum to afford 1.181 g (84%) (S)-3-(1H-indol-3-yl)-2-{(S)-2-[(3-methyl-1H-indene-2-carbonyl)-amino]-propionylamino}-propionic acid tert-butyl ester.

A microwave vial was charged with (S)-3-(1H-indol-3-yl)-2-{(S)-2-[(3-methyl-1H-indene-2-carbonyl)-amino]-propionylamino}-propionic acid tert-butyl ester (0.120 g, 0.25 mmol) and 1,1,1,3,3,3-hexafluoro-2-propanol (2.0 ml, 19.0 mmol). The vial was flushed with argon and sealed. The colorless solution was heated at 120° C. for 2 h under microwave irradiation.

The reaction mixture was concentrated to afford (S)-3-(1H-indol-3-yl)-2-{(S)-2-[(3-methyl-1H-indene-2-carbonyl)-amino]-propionylamino}-propionic acid as an off-white foam and used without further purification

In a 10 ml round-bottomed flask, [(S)-1-(benzylcarbamoyl-hydroxy-methyl)-pentyl]-carbamic acid tert-butyl ester (100 mg, 0.29 mmol) was dissolved in 3.0 ml dichloromethane. Trifluoroacetic acid (0.5 ml, 6.49 mmol) was added slowly. The reaction mixture was stirred at room temperature for 2.5 h. The solvent was evaporated and then put under high vacuum for 15 min. The residue was dissolved in 1.0 ml DMF and the solution was stirred at 0° C. for 5 min. N,N-Diisopropylethylamine (185 mg, 0.25 ml, 1.43 mmol) was added dropwise at 0° C. (S)-3-(1H-Indol-3-yl)-2-{(S)-2-[(3-methyl-1H-indene-2-carbonyl)-amino]-propionylamino}-propionic acid (132 mg, 0.23 mmol; purity=75%), dissolved in 1.0 ml DMF was added followed by HATU (96 mg, 0.25 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then put under high vacuum to afford 121 mg (79%) 3-methyl-1H-indene-2-carboxylic acid {(S)-1-[(S)-1-[(S)-1-(benzylcarbamoyl-hydroxy-methyl)-pentylcarbamoyl]-2-(1H-indol-3-yl)-ethylcarbamoyl]-ethyl}-amide as a mixture of epimers.

In a 25 ml round-bottomed flask, 3-methyl-1H-indene-2-carboxylic acid {(S)-1-[(S)-1-[(S)-1-(benzylcarbamoyl-hydroxy-methyl)-pentylcarbamoyl]-2-(1H-indol-3-yl)-ethylcarbamoyl]-ethyl}-amide (118 mg, 0.18 mmol) was dissolved in 6.5 ml dichloromethane and Dess-Martin periodinane (113 mg, 0.27 mmol) was added. The reaction mixture was stirred at room temperature for 3 h (a precipitate was formed). The reaction mixture was quenched with 3 ml saturated NaHCO₃-solution and 3 ml 10% Na₂S₂O₃-solution and stirred vigorously for 1 h at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue (44 mg brown solid) was triturated with dichloromethane/hexanes to afford 28 mg (23%) 3-methyl-1H-indene-2-carboxylic acid {(S)-1-[(S)-1-((S)-1-benzylaminooxalyl-pentylcarbamoyl)-2-(1H-indol-3-yl)-ethylcarbamoyl]-ethyl}-amide as a light brown solid. LC/MS: (M−H)⁻=660.

### Example 6

### 5-Methyl-isoxazole-3-carboxylic acid {(S)-1-[(S)-1-((S)-1-benzylaminooxalyl-pentylcarbamoyl)-2-methoxy-ethylcarbamoyl]-ethyl}-amide

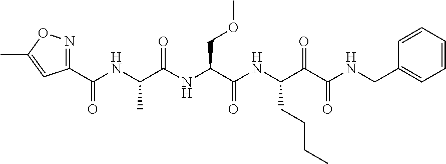 [SMILES: CCCC[C@H](NC(=O)[C@H](COC)NC(=O)[C@H](C)NC(=O)c1cc(C)on1)C(=O)C(=O)NCc1ccccc1]

In a 250 ml 3-neck round-bottomed flask, Boc-O-methyl-L-serine dicyclohexylammonium salt (1.800 g, 4.49 mmol) was dissolved in 55 ml dichloromethane. The colorless solution was cooled to 0° C. Triethylamine (1.02 g, 1.4 ml, 10.0 mmol) was added at 0° C. followed by 4-dimethylaminopyridine (80 mg, 0.65 mmol). A solution of benzyl chloroformate (1.67 g, 1.4 ml, 9.81 mmol) in 10 ml dichloromethane was added dropwise (the reaction mixture turned into a yellow suspension). The reaction mixture was stirred at 0° C. for 2 h and at room temperature overnight. The reaction mixture was quenched with 20 ml saturated NaHCO₃-solution and then extracted with 50 ml dichloromethane and 10 ml water. The organic layer was washed with 20 ml saturated NaHCO₃-solution. The aqueous layers were extracted with 100 ml dichloromethane. The organic layers were combined, dried over sodium sulfate, filtered and concentrated. The residue (yellow oil) was chromatographed over 80 g silica gel with EtOAc/hexanes (gradient 0-20% EtOAc). All fractions containing product were combined and concentrated to afford 1.223 g (88%) (S)-2-tert-butoxycarbonylamino-3-methoxy-propionic acid benzyl ester as a light yellow oil.

(S)-2-tert-Butoxycarbonylamino-3-methoxy-propionic acid benzyl ester (1.22 g, 3.94 mmol) was dissolved in 13 ml dichloromethane. Trifluoroacetic acid (8.5 ml, 110 mmol) was added slowly. The reaction mixture was stirred at room temperature for 2.5 h. The solvent was evaporated and then put under high vacuum. The residue (light yellow oil) was dissolved in 12 ml DMF and the solution was stirred at 0° C. for 5 min. N,N-Diisopropylethylamine (3.7 g, 5.0 ml, 28.6 mmol) was added dropwise at 0° C. Boc-L-alanine (641 mg, 3.39 mmol) was added followed by HATU (1.42 g, 3.73 mmol). After the addition was complete, the ice bath was removed and the reaction mixture was stirred at room temperature overnight. The reaction mixture was extracted with 100 ml diethyl ether and 10 ml water. The aqueous layer was extracted with 100 ml diethyl ether. The organic layers were washed three times with 10 ml water and once with 10 ml brine. The organic layers were combined, dried over sodium sulfate, filtered and concentrated to afford (S)-2-((S)-2-tert-butoxycarbonylamino-propionylamino)-3-methoxy-propionic acid benzyl ester as a yellow oil which was used without further purification.

In a 50 ml round-bottomed flask, (S)-2-((S)-2-tert-butoxycarbonylamino-propionylamino)-3-methoxy-propionic acid benzyl ester (863 mg, 1.7 mmol; purity=75%) was dissolved in 6.0 ml dichloromethane. Trifluoroacetic acid (3.0 ml, 38.9 mmol) was added slowly. The reaction mixture was stirred at room temperature for 3 h. The solvent was evaporated and then put under high vacuum for 30 min. The residue was dissolved in 5.0 ml DMF and the solution was stirred at 0° C. for 5 min. N,N-Diisopropylethylamine (1.48 g, 2.0 ml, 11.5 mmol) was added dropwise at 0° C. 5-Methylisoxazole-3-carboxylic acid (190 mg, 1.49 mmol) was added followed by HATU (625 mg, 1.64 mmol). After the addition was complete, the ice bath was removed and the reaction mixture was stirred at room temperature overnight. The reaction mixture was extracted with 70 ml diethyl ether and 5 ml water. The aqueous layer was extracted with 70 ml diethyl ether. The organic layers were washed twice with 5 ml water and once with 5 ml brine. The organic layers were combined, dried over sodium sulfate, filtered and concentrated. The residue was chromatographed over 25 g silica gel with EtOAc/Hexanes (gradient 0-70% EtOAc). All fractions containing product were combined and concentrated to afford 571 mg (93%) (S)-3-methoxy-2-{(S)-2-[(5-methyl-isoxazole-3-carbonyl)-amino]-propionylamino}-propionic acid benzyl ester as a light yellow oil.

In a 50 ml round-bottomed flask, (S)-3-methoxy-2-{(S)-2-[(5-methyl-isoxazole-3-carbonyl)-amino]-propionylamino}-propionic acid benzyl ester (565 mg, 1.38 mmol) was dissolved in 12 ml methanol. The flask was three times alternating evacuated and flushed with argon. 20% Palladium hydroxide on carbon (125 mg, 0.18 mmol) was added carefully. The flask was evacuated, flushed with argon, evacuated and flushed with hydrogen. The reaction mixture was stirred under hydrogen atmosphere (balloon) at room temperature for 2 h. The reaction mixture was filtered over Celite, rinsing with ethyl acetate/methanol. The filtrate was concentrated to afford 427 mg (93%; purity=90%) (S)-3-methoxy-2-{(S)-2-[(5-methyl-isoxazole-3-carbonyl)-amino]-propionylamino}-propionic acid as a light yellow foam.

In a 10 ml round-bottomed flask, [(S)-1-(benzylcarbamoyl-hydroxy-methyl)-pentyl]-carbamic acid tert-butyl ester (100 mg, 0.29 mmol) was dissolved in 3.0 ml dichloromethane. Trifluoroacetic acid (0.5 ml, 6.49 mmol) was added slowly. The reaction mixture was stirred at room temperature for 2.5 h. The solvent was evaporated and then put under high vacuum for 30 min. The residue was dissolved in 2.0 ml DMF and the solution was stirred at 0° C. for 5 min. N,N-Diisopropylethylamine (185 mg, 0.25 ml, 1.43 mmol) was added dropwise at 0° C. (S)-3-Methoxy-2-{(S)-2-[(5-methyl-isoxazole-3-carbonyl)-amino]-propionylamino}-propionic acid (79 mg, 0.24 mmol; purity=90%) was added followed by HATU (99 mg, 0.26 mmol). After the addition was complete, the ice bath was removed and the reaction mixture was stirred at room temperature overnight. The reaction mixture was extracted with 40 ml diethyl ether/EtOAc (1:1) and 3 ml water. The aqueous layer was extracted with 40 ml diethyl ether/EtOAc (1:1). The organic layers were washed twice with 3 ml water and once with 3 ml brine. The organic layers were combined, dried over sodium sulfate, filtered and concentrated. The residue was chromatographed over 12 g silica gel with MeOH/dichloromethane (gradient 0-5% MeOH). All fractions containing product were combined and concentrated to afford 80 mg (63%) 5-methyl-isoxazole-3-carboxylic acid ((S)-1-{(S)-1-[(S)-1-(benzylcarbamoyl-hydroxy-methyl)-pentylcarbamoyl]-2-methoxy-ethylcarbamoyl}-ethyl)-amide as an off-white solid and as a mixture of epimers.

In a 25 ml round-bottomed flask, 5-methyl-isoxazole-3-carboxylic acid ((S)-1-{(S)-1-[(S)-1-(benzylcarbamoyl-hydroxy-methyl)-pentylcarbamoyl]-2-methoxy-ethylcarbamoyl}-ethyl)-amide (77 mg, 0.15 mmol) was dissolved in 5.2 ml dichloromethane and Dess-Martin periodinane (92 mg, 0.22 mmol) was added. The reaction mixture was stirred at room temperature for 3 h. The reaction mixture was quenched with 3 ml saturated NaHCO₃-solution and 3 ml 10% Na₂S₂O₃-solution and stirred vigorously for 30 min at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was chromatographed over 12 g silica gel with MeOH/dichloromethane (gradient 0-5% MeOH). All fractions containing product were combined and concentrated to afford 48 mg (59%) 5-methyl-isoxazole-3-carboxylic acid {(S)-1-[(S)-1-((S)-1-benzylaminooxalyl-pentylcarbamoyl)-2-methoxy-ethylcarbamoyl]-ethyl}-amide as an off-white solid and as a mixture of epimers (ratio 6:1; based on NMR). LC/MS: (M+H)⁺=530.

### Example 7

### 3-Methyl-1H-indene-2-carboxylic acid OS)-1-{(S)-2-(1H-indol-3-yl)-1-[(S)-1-((S)-1-phenyl-ethylaminooxalyl)-pentylcarbamoyl]-ethylcarbamoyl}-ethyl)-amide

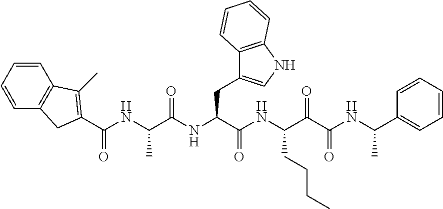 [SMILES: CCCC[C@H](NC(=O)[C@H](Cc1c[nH]c2ccccc12)NC(=O)[C@H](C)NC(=O)C1=C(C)c2ccccc2C1)C(=O)C(=O)N[C@@H](C)c1ccccc1]

A 10 ml round-bottomed flask was charged with (S)-3-tert-butoxycarbonylamino-2-hydroxy-heptanoic acid (200 mg, 0.77 mmol), (S)-1-phenylethanamine (124 mg, 0.13 ml, 1.02 mmol) and 3.0 ml DMF. N,N-Diisopropylethylamine (244 mg, 0.33 ml, 1.89 mmol) was added followed by HATU (320 mg, 0.84 mmol). The yellow solution was stirred at room temperature overnight. The reaction mixture was extracted with 40 ml diethyl ether/EtOAc (1:1) and 3 ml water. The aqueous layer was extracted with 40 ml diethyl ether/EtOAc (1:1). The organic layers were washed twice with 3 ml water and once with 3 ml brine. The organic layers were combined, dried over sodium sulfate, filtered and concentrated. The residue was chromatographed over 25 g silica gel with EtOAc/hexanes (gradient 0-30% EtOAc). All fractions containing product were combined and concentrated to afford 148 mg (53%) {(S)-1-[hydroxy-((S)-1-phenyl-ethylcarbamoyl)-methyl]-pentyl}-carbamic acid tert-butyl ester as an off-white solid and as a mixture of epimers.

In a 10 ml round-bottomed flask, {(S)-1-[hydroxy-((S)-1-phenyl-ethylcarbamoyl)-methyl]-pentyl}-carbamic acid tert-butyl ester (135 mg, 0.37 mmol) was dissolved in 3.7 ml dichloromethane. Trifluoroacetic acid (0.65 ml, 8.44 mmol) was added slowly. The reaction mixture was stirred at room temperature for 2.5 h. The solvent was evaporated and then put under high vacuum for 30 min. The residue was dissolved in 2.5 ml DMF and the solution was stirred at 0° C. for 5 min. N,N-Diisopropylethylamine (259 mg, 0.35 ml, 2.00 mmol) was added dropwise at 0° C. (S)-3-(1H-Indol-3-yl)-2-{(S)-2-[(3-methyl-1H-indene-2-carbonyl)-amino]-propionylamino}-propionic acid (0.180 g, 0.29 mmol; purity=70%) was added followed by HATU (122 mg, 0.32 mmol). After the addition was complete, the ice bath was removed and the reaction mixture was stirred at room temperature overnight. The reaction mixture was extracted with 40 ml diethyl ether/EtOAc (1:1) and 3 ml water. The aqueous layer was extracted with 40 ml diethyl ether/EtOAc (1:1). The organic layers were washed twice with 3 ml water and once with 3 ml brine. The organic layers were combined and concentrated. The residue was triturated with petroleum ether to afford 274 mg (97%; purity=70%) 3-methyl-1H-indene-2-carboxylic acid {(S)-1-[(S)-1-{(S)-1-[hydroxy-((S)-1-phenyl-ethylcarbamoyl)-methyl]-pentylcarbamoyl}-2-(1H-indol-3-yl)-ethylcarbamoyl]-ethyl}-amide as a light yellow solid and a mixture of epimers.

In a 50 ml round-bottomed flask, 3-methyl-1H-indene-2-carboxylic acid {(S)-1-[(S)-1-{(S)-1-[hydroxy-((S)-1-phenyl-ethylcarbamoyl)-methyl]-pentylcarbamoyl}-2-(1H-indol-3-yl)-ethylcarbamoyl]-ethyl}-amide (272 mg, 0.28 mmol; purity=70%) was dissolved in 10 ml dichloromethane and Dess-Martin periodinane (179 mg, 0.42 mmol) was added. The reaction mixture was stirred at room temperature for 3 h. The reaction mixture was quenched with 4 ml saturated NaHCO₃-solution and 4 ml 10% Na₂S₂O₃-solution and stirred vigorously for 1 h at room temperature. The biphasic mixture was then extracted with 30 ml Dichloromethane. The organic layer was washed with 5 ml saturated NaHCO₃-solution. The aqueous layers were extracted twice with 30 ml Dichloromethane. The organic layers were combined, dried over sodium sulfate, filtered and concentrated. The residue was chromatographed over 12 g silica gel with MeOH/dichloromethane (gradient 0-5% MeOH). All fractions containing product were combined and concentrated. The residue (brown oil) was triturated with dichloromethane/hexanes to afford 15 mg (7%; purity=90%) 3-methyl-1H-indene-2-carboxylic acid ((S)-1-{(S)-2-(1H-indol-3-yl)-1-[(S)-1-((S)-1-phenyl-ethylaminooxalyl)-pentylcarbamoyl]-ethylcarbamoyl}-ethyl)-amide as a light brown solid. LC/MS: (M−H)⁻=674.

### Example 8

### 3-Methyl-1H-indene-2-carboxylic acid 08)-1-{(S)-2-(1H-indol-3-yl)-1-[(S)-1-((R)-1-phenyl-ethylaminooxalyl)-pentylcarbamoyl]-ethylcarbamoyl}-ethyl)-amide

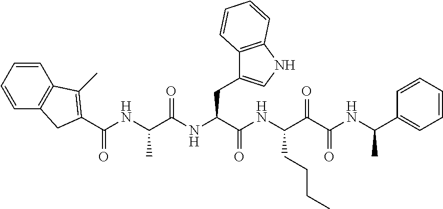 [SMILES: CCCC[C@H](NC(=O)[C@H](Cc1c[nH]c2ccccc12)NC(=O)[C@H](C)NC(=O)C1=C(C)c2ccccc2C1)C(=O)C(=O)N[C@H](C)c1ccccc1]

A 10 ml round-bottomed flask was charged with (S)-3-tert-butoxycarbonylamino-2-hydroxy-heptanoic acid (200 mg, 0.77 mmol), (R)-1-phenylethanamine (124 mg, 0.13 ml, 1.02 mmol) and 3.0 ml DMF. N,N-Diisopropylethylamine (244 mg, 0.33 ml, 1.89 mmol) was added followed by HATU (320 mg, 0.84 mmol). The yellow solution was stirred at room temperature overnight. The reaction mixture was extracted with 40 ml diethyl ether/EtOAc (1:1) and 3 ml water. The aqueous layer was extracted with 40 ml diethyl ether/EtOAc (1:1). The organic layers were washed twice with 3 ml water and once with 3 ml brine. The organic layers were combined, dried over sodium sulfate, filtered and concentrated. The residue was chromatographed over 25 g silica gel with EtOAc/hexanes (gradient 0-30% EtOAc). All fractions containing product were combined and concentrated to afford 123 mg (44%) {(S)-1-[hydroxy-((R)-1-phenyl-ethylcarbamoyl)-methyl]-pentyl}-carbamic acid tert-butyl ester as an off-white solid and as a mixture of epimers.

In a 10 ml round-bottomed flask, {(S)-1-[hydroxy-((R)-1-phenyl-ethylcarbamoyl)-methyl]-pentyl}-carbamic acid tert-butyl ester (121 mg, 0.33 mmol) was dissolved in 3.3 ml dichloromethane. Trifluoroacetic acid (0.58 ml, 7.53 mmol) was added slowly. The reaction mixture was stirred at room temperature for 2.5 h. The solvent was evaporated and then put under high vacuum for 30 min. The residue was dissolved in 2.5 ml DMF and the solution was stirred at 0° C. for 5 min. N,N-Diisopropylethylamine (222 mg, 0.30 ml, 1.72 mmol) was added dropwise at 0° C. (S)-3-(1H-Indol-3-yl)-2-{(S)-2-[(3-methyl-1H-indene-2-carbonyl)-amino]-propionylamino}-propionic acid (160 mg, 0.26 mmol; purity=70%) was added followed by HATU (109 mg, 0.29 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then put under high vacuum to afford 136 mg (77%) 3-methyl-1H-indene-2-carboxylic acid {(S)-1-[(S)-1-{(S)-1-[hydroxy-((R)-1-phenyl-ethylcarbamoyl)-methyl]-pentylcarbamoyl}-2-(1H-indol-3-yl)-ethylcarbamoyl]-ethyl}-amide as a mixture of epimers.

In a 50 ml round-bottomed flask, 3-methyl-1H-indene-2-carboxylic acid {(S)-1-[(S)-1-{(S)-1-[hydroxy-((R)-1-phenyl-ethylcarbamoyl)-methyl]-pentylcarbamoyl}-2-(1H-indol-3-yl)-ethylcarbamoyl]-ethyl}-amide (134 mg, 0.20 mmol) was dissolved in 7.0 ml dichloromethane and Dess-Martin periodinane (126 mg, 0.30 mmol) was added. The reaction mixture was stirred at room temperature for 3 h. The reaction mixture was quenched with 4 ml saturated NaHCO₃-solution and 4 ml 10% Na₂S₂O₃-solution and stirred vigorously for 1.5 h at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was chromatographed over 12 g silica gel MeOH/dichloromethane (gradient 0-5% MeOH). All fractions containing product were combined and concentrated. The residue was triturated with dichloromethane/hexanes to afford 28 mg (20%) 3-methyl-1H-indene-2-carboxylic acid ((S)-1-{(S)-2-(1H-indol-3-yl)-1-[(S)-1-((R)-1-phenyl-ethylaminooxalyl)-pentylcarbamoyl]-ethylcarbamoyl}-ethyl)-amide as a light yellow solid. LC/MS: (M−H)⁻=674.

### Example 9

### {(S)-1-[(S)-1-((S)-1-Benzylaminooxalyl-pentylcarbamoyl)-2-(1H-indol-3-yl)-ethylcarbamoyl]-ethyl}-carbamic acid benzyl ester

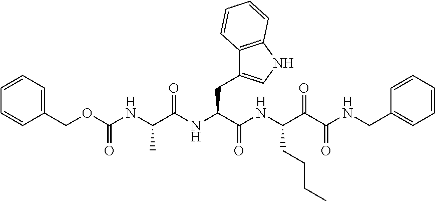 [SMILES: CCCC[C@H](NC(=O)[C@H](Cc1c[nH]c2ccccc12)NC(=O)[C@H](C)NC(=O)OCc1ccccc1)C(=O)C(=O)NCc1ccccc1]

A microwave vial was charged with (S)-2-((S)-2-benzyloxycarbonylamino-propionylamino)-3-(1H-indol-3-yl)-propionic acid tert-butyl ester (145 mg, 0.26 mmol; purity=85%) and 1,1,1,3,3,3-hexafluoro-2-propanol (2.2 ml, 20.9 mmol). The vial was flushed with argon and sealed. The colorless solution was heated at 120° C. for 2 h under microwave irradiation. The reaction mixture was concentrated to afford (S)-2-((S)-2-benzyloxycarbonylamino-propionylamino)-3-(1H-indol-3-yl)-propionic acid as an off-white foam which was used without further purification.

In a 10 ml round-bottomed flask, [(S)-1-(benzylcarbamoyl-hydroxy-methyl)-pentyl]-carbamic acid tert-butyl ester (95 mg, 0.27 mmol) was dissolved in 3.0 ml dichloromethane. Trifluoroacetic acid (0.5 ml, 6.49 mmol) was added slowly. The reaction mixture was stirred at room temperature for 2.5 h. The solvent was evaporated and then placed under high vacuum for 30 min. The residue was dissolved in 1.0 ml DMF and the solution was stirred at 0° C. for 5 min. N,N-Diisopropylethylamine (185 mg, 0.25 ml, 1.43 mmol) was added dropwise at 0° C. (S)-2-((S)-2-Benzyloxycarbonylamino-propionylamino)-3-(1H-indol-3-yl)-propionic acid (161 mg, 0.24 mmol; purity=60%), dissolved in 1.0 ml DMF was added followed by HATU (99 mg, 0.26 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then placed under high vacuum to afford 134 mg (89%) {(S)-1-[(S)-1-[(S)-1-(benzylcarbamoyl-hydroxy-methyl)-pentylcarbamoyl]-2-(1H-indol-3-yl)-ethylcarbamoyl]-ethyl}-carbamic acid benzyl ester as a mixture of epimers.

In a 25 ml round-bottomed flask, {(S)-1-[(S)-1-[(S)-1-(benzylcarbamoyl-hydroxy-methyl)-pentylcarbamoyl]-2-(1H-indol-3-yl)-ethylcarbamoyl]-ethyl}-carbamic acid benzyl ester (132 mg, 0.21 mmol) was dissolved in 7.5 ml dichloromethane and Dess-Martin periodinane (131 mg, 0.31 mmol) was added. The reaction mixture was stirred at room temperature for 2.5 h. The reaction mixture was quenched with 4 ml saturated NaHCO₃-solution and 4 ml 10% Na₂S₂O₃-solution and stirred vigorously for 1 h at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue was triturated with dichloromethane/hexanes to afford 35 mg (24%; purity=90%) {(S)-1-[(S)-1-((S)-1-benzylaminooxalyl-pentylcarbamoyl)-2-(1H-indol-3-yl)-ethylcarbamoyl]-ethyl}-carbamic acid benzyl ester as a light brown solid. LC/MS: (M+H)⁺=640.

### Example 10

### (S)-3-{(S)-3-(1H-Indol-3-yl)-2-[(S)-2-(2-morpholin-4-yl-acetylamino)-propionylamino]-propionylamino}-2-oxo-heptanoic acid benzylamide

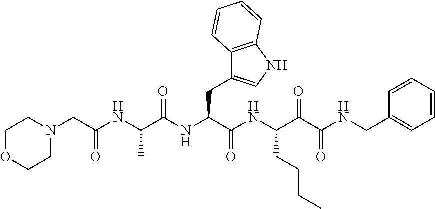 [SMILES: CCCC[C@H](NC(=O)[C@H](Cc1c[nH]c2ccccc12)NC(=O)[C@H](C)NC(=O)CN1CCOCC1)C(=O)C(=O)NCc1ccccc1]

In a 50 ml round-bottomed flask, 2-morpholinoacetic acid (275 mg, 1.89 mmol) and (S)-2-((S)-2-amino-propionylamino)-3-(1H-indol-3-yl)-propionic acid tert-butyl ester (900 mg, 2.17 mmol; purity=80%) were dissolved in 6.6 ml DMF. The reaction mixture was cooled to 0° C. N,N-Diisopropylethylamine (740 mg, 1.0 ml, 5.73 mmol) was added slowly at 0° C. followed by HATU (792 mg, 2.08 mmol). The yellow solution was stirred at room temperature overnight. The reaction mixture was extracted with dichloromethane and water. The organic layer was washed with water. The aqueous layers were extracted twice with dichloromethane. The organic layers were combined, dried over sodium sulfate, filtered, concentrated and then placed under high vacuum. The residue was chromatographed over 40 g silica gel with MeOH/dichloromethane (gradient 0-5% MeOH). All fractions containing product were combined and concentrated to afford 937 mg (97%; purity=90%) (S)-3-(1H-indol-3-yl)-2-[(S)-2-(2-morpholin-4-yl-acetylamino)-propionylamino]-propionic acid tert-butyl ester as an off-white foam.

A microwave vial was charged with (S)-3-(1H-indol-3-yl)-2-[(S)-2-(2-morpholin-4-yl-acetylamino)-propionylamino]-propionic acid tert-butyl ester (135 mg, 0.27 mmol; purity=90%) and 1,1,1,3,3,3-hexafluoro-2-propanol (2.2 ml, 20.9 mmol). The vial was flushed with argon and sealed. The colorless solution was heated at 120° C. for 2 h under microwave irradiation (the reaction mixture turned into a yellow solution). The reaction mixture was concentrated to afford (S)-3-(1H-indol-3-yl)-2-[(S)-2-(2-morpholin-4-yl-acetylamino)-propionylamino]-propionic acid as a yellow foam which was used without further purification.

In a 10 ml round-bottomed flask, [(S)-1-(benzylcarbamoyl-hydroxy-methyl)-pentyl]-carbamic acid tert-butyl ester (100 mg, 0.29 mmol) was dissolved in 3.0 ml dichloromethane. Trifluoroacetic acid (0.5 ml, 6.49 mmol) was added slowly. The reaction mixture was stirred at room temperature for 2.5 h. The solvent was evaporated and then put under high vacuum for 15 min. The residue and (S)-3-(1H-indol-3-yl)-2-[(S)-2-(2-morpholin-4-yl-acetylamino)-propionylamino]-propionic acid (205 mg, 0.25 mmol; purity=50%) were dissolved in 2.0 ml dichloromethane and cooled to 0° C. N,N-Diisopropylethylamine (222 mg, 0.30 ml, 1.72 mmol) was added dropwise at 0° C. followed by HATU (107 mg, 0.28 mmol). After the addition was complete, the ice bath was removed and the reaction mixture was stirred at room temperature overnight. The reaction mixture was extracted with 3 ml water and 30 ml dichloromethane. The organic layer was washed with 3 ml water. The aqueous layers were extracted twice with 30 ml dichloromethane. The organic layers were combined, dried over sodium sulfate, filtered and concentrated. The residue was chromatographed over 12 g silica gel with MeOH/dichloromethane (gradient 0-10% MeOH). All fractions containing product were combined and concentrated to afford 100 mg (62%) (S)-2-hydroxy-3-{(S)-3-(1H-indol-3-yl)-2-[(S)-2-(2-morpholin-4-yl-acetylamino)-propionylamino]-propionylamino}-heptanoic acid benzylamide as a yellow solid and a mixture of epimers.

In a 25 ml round-bottomed flask, (S)-2-hydroxy-3-{(S)-3-(1H-indol-3-yl)-2-[(S)-2-(2-morpholin-4-yl-acetylamino)-propionylamino]-propionylamino}-heptanoic acid benzylamide (89 mg, 0.140 mmol) was dissolved in 5.2 ml dichloromethane and Dess-Martin periodinane (90 mg, 0.21 mmol) was added. The reaction mixture was stirred at room temperature for 2.5 h (the reaction mixture turned dark brown). The reaction mixture was quenched with 3 ml saturated NaHCO₃-solution and 3 ml 10% Na₂S₂O₃-solution and stirred vigorously for 30 min at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was chromatographed over 12 g silica gel with MeOH/dichloromethane (gradient 0-5% MeOH). All fractions containing product were combined and concentrated to afford 18 mg (19%) (S)-3-{(S)-3-(1H-indol-3-yl)-2-[(S)-2-(2-morpholin-4-yl-acetylamino)-propionylamino]-propionylamino}-2-oxo-heptanoic acid benzylamide as a light brown foam. LC/MS: (M+H)⁺=633.

### Example 11

### Indan-2-carboxylic acid {(S)-1-[(S)-1-((S)-1-benzylaminooxalyl-pentylcarbamoyl)-2-(1H-indol-3-yl)-ethylcarbamoyl]-ethyl}-amide

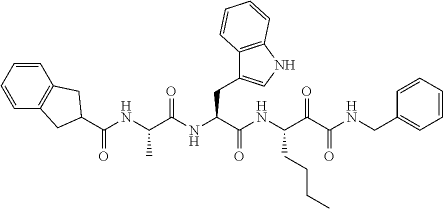 [SMILES: CCCC[C@H](NC(=O)[C@H](Cc1c[nH]c2ccccc12)NC(=O)[C@H](C)NC(=O)C1Cc2ccccc2C1)C(=O)C(=O)NCc1ccccc1]

In a 50 ml round-bottomed flask, (S)-2-((S)-2-amino-propionylamino)-3-(1H-indol-3-yl)-propionic acid tert-butyl ester (800 mg, 1.93 mmol; purity=80%) was dissolved in 5.9 ml DMF. The pale yellow solution was cooled to 0° C. N,N-Diisopropylethylamine (666 mg, 0.90 ml, 5.15 mmol) was added slowly at 0° C. 2-Indanecarboxylic acid (275 mg, 1.7 mmol) was added followed by HATU (709 mg, 1.87 mmol). After the addition was complete, the ice bath was removed and the reaction mixture was stirred at room temperature for 3 days. The reaction mixture was extracted with 70 ml diethyl ether and 10 ml water. The aqueous layer was extracted with 70 ml diethyl ether. The organic layers were washed twice with 10 ml water and once with 10 ml brine. The organic layers were combined, dried over sodium sulfate, filtered and concentrated. The residue (light yellow oil) was chromatographed over 40 g silica gel with EtOAc/hexanes (gradient 0-60% EtOAc). All fractions containing product were combined and concentrated to afford 761 mg (90%) (S)-2-{(S)-2-[(indane-2-carbonyl)-amino]-propionylamino}-3-(1H-indol-3-yl)-propionic acid tert-butyl ester as an off-white foam.

A microwave vial was charged with (S)-2-{(S)-2-[(indane-2-carbonyl)-amino]-propionylamino}-3-(1H-indol-3-yl)-propionic acid tert-butyl ester (136 mg, 0.27 mmol) and 1,1,1,3,3,3-hexafluoro-2-propanol (2.2 ml, 20.9 mmol). The vial was flushed with argon and sealed. The colorless solution was heated at 120° C. for 2 h under microwave irradiation. The reaction mixture was concentrated to afford (S)-2-{(S)-2-[(indane-2-carbonyl)-amino]-propionylamino}-3-(1H-indol-3-yl)-propionic acid as a light brown oil which was used without further purification.

In a 10 ml round-bottomed flask, [(S)-1-(benzylcarbamoyl-hydroxy-methyl)-pentyl]-carbamic acid tert-butyl ester (110 mg, 0.31 mmol) was dissolved in 3.0 ml dichloromethane. Trifluoroacetic acid (0.55 ml, 7.14 mmol) was added slowly. The reaction mixture was stirred at room temperature for 2.5 h. The solvent was evaporated and then placed under high vacuum for 15 min. The residue was dissolved in 1.0 ml DMF and the solution was stirred at 0° C. for 5 min. N,N-Diisopropylethylamine (222 mg, 0.30 ml, 1.72 mmol) was added dropwise at 0° C. (S)-2-{(S)-2-[(indane-2-carbonyl)-amino]-propionylamino}-3-(1H-indol-3-yl)-propionic acid (210 mg, 0.25 mmol; purity=50%), dissolved in 1.1 ml DMF was added followed by HATU (105 mg, 0.28 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then placed under high vacuum to afford 157 mg (96%) indan-2-carboxylic acid {(S)-1-[(S)-1-[(S)-1-(benzylcarbamoyl-hydroxy-methyl)-pentylcarbamoyl]-2-(1H-indol-3-yl)-ethylcarbamoyl]-ethyl}-amide as a mixture of epimers.

In a 25 ml round-bottomed flask, indan-2-carboxylic acid {(S)-1-[(S)-1-[(S)-1-(benzylcarbamoyl-hydroxy-methyl)-pentylcarbamoyl]-2-(1H-indol-3-yl)-ethylcarbamoyl]-ethyl}-amide (151 mg, 0.23 mmol) was partially dissolved in 8.5 ml dichloromethane and Dess-Martin periodinane (147 mg, 0.35 mmol) was added. The reaction mixture was stirred at room temperature for 2.5 h. The reaction mixture was quenched with 4 ml saturated NaHCO₃-solution and 4 ml 10% Na₂S₂O₃-solution and stirred vigorously for 30 min at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was chromatographed over 12 g silica gel with MeOH/dichloromethane (gradient 0-5% MeOH). All fractions containing product were combined and concentrated. The residue was triturated with dichloromethane/hexanes to afford 51 mg (30%; purity=90%) indan-2-carboxylic acid {(S)-1-[(S)-1-((S)-1-benzylaminooxalyl-pentylcarbamoyl)-2-(1H-indol-3-yl)-ethylcarbamoyl]-ethyl}-amide as a brown solid. LC/MS: (M−H)⁻=648.

### Example 12

### N-{(S)-1-[(S)-1-((S)-1-Benzylaminooxalyl-pentylcarbamoyl)-2-(1H-indol-3-yl)-ethylcarbamoyl]-ethyl}-2,3-dichloro-benzamide

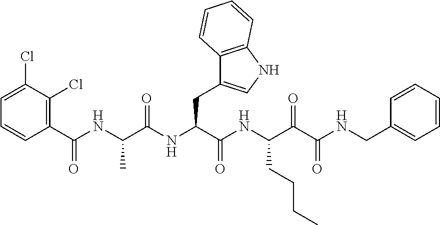 [SMILES: CCCC[C@H](NC(=O)[C@H](Cc1c[nH]c2ccccc12)NC(=O)[C@H](C)NC(=O)c1cccc(Cl)c1Cl)C(=O)C(=O)NCc1ccccc1]

In a 50 ml round-bottomed flask, (S)-2-((S)-2-amino-propionylamino)-3-(1H-indol-3-yl)-propionic acid tert-butyl ester (800 mg, 1.93 mmol; purity=80%) was dissolved in 5.9 ml DMF. The pale yellow solution was cooled to 0° C. N,N-Diisopropylethylamine (666 mg, 0.9 ml, 5.15 mmol) was added slowly at 0° C. 2,3-Dichlorobenzoic acid (325 mg, 1.7 mmol) was added followed by HATU (712 mg, 1.87 mmol). After the addition was complete, the ice bath was removed and the reaction mixture was stirred at room temperature overnight. The reaction mixture was extracted with 70 ml diethyl ether and 10 ml water. The aqueous layer was extracted with 70 ml diethyl ether. The organic layers were washed twice with 10 ml water and once with 10 ml brine. The organic layers were combined, dried over sodium sulfate, filtered and concentrated. The residue (light yellow oil) was chromatographed over 40 g silica gel with EtOAc/Hexanes (gradient 0-40% EtOAc). All fractions containing product were combined and concentrated to afford 794 mg (93%) (S)-2-[(S)-2-(2,3-dichloro-benzoylamino)-propionylamino]-3-(1H-indol-3-yl)-propionic acid tert-butyl ester as an off-white foam.

A microwave vial was charged with (S)-2-[(S)-2-(2,3-dichloro-benzoylamino)-propionylamino]-3-(1H-indol-3-yl)-propionic acid tert-butyl ester (137 mg, 0.27 mmol) and 1,1,1,3,3,3-hexafluoro-2-propanol (2.2 ml, 20.9 mmol). The vial was flushed with argon and sealed. The colorless solution was heated at 120° C. for 2 h under microwave irradiation. The reaction mixture was concentrated to afford (S)-2-[(S)-2-(2,3-dichloro-benzoylamino)-propionylamino]-3-(1H-indol-3-yl)-propionic acid as an off-white foam which was used without further purification.

In a 10 ml round-bottomed flask, [(S)-1-(benzylcarbamoyl-hydroxy-methyl)-pentyl]-carbamic acid tert-butyl ester (110 mg, 0.31 mmol) was dissolved in 3.0 ml dichloromethane. Trifluoroacetic acid (0.55 ml, 7.14 mmol) was added slowly. The reaction mixture was stirred at room temperature for 2.5 h. The solvent was evaporated and then put under high vacuum for 15 min. The residue was dissolved in 1.0 ml DMF and the solution was stirred at 0° C. for 5 min. N,N-Diisopropylethylamine (222 mg, 0.30 ml, 1.72 mmol) was added dropwise at 0° C. (S)-2-[(S)-2-(2,3-Dichloro-benzoylamino)-propionylamino]-3-(1H-indol-3-yl)-propionic acid (149 mg, 0.27 mmol; purity=80%), dissolved in 1.1 ml DMF was added followed by HATU (111 mg, 0.29 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then placed under high vacuum to provide 158 mg (87%) N-{(S)-1-[(S)-1-[(S)-1-(benzylcarbamoyl-hydroxy-methyl)-pentylcarbamoyl]-2-(1H-indol-3-yl)-ethylcarbamoyl]-ethyl}-2,3-dichloro-benzamide as a mixture of epimers.

In a 25 ml round-bottomed flask, N-{(S)-1-[(S)-1-[(S)-1-(benzylcarbamoyl-hydroxy-methyl)-pentylcarbamoyl]-2-(1H-indol-3-yl)-ethylcarbamoyl]-ethyl}-2,3-dichloro-benzamide (152 mg, 0.22 mmol) was partially dissolved in 8.0 ml dichloromethane and Dess-Martin periodinane (142 mg, 0.33 mmol) was added. The reaction mixture was stirred at room temperature for 2.5 h. The reaction mixture was quenched with 3.5 ml saturated NaHCO₃-solution and 3.5 ml 10% Na₂S₂O₃-solution and stirred vigorously for 30 min at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was chromatographed over 12 g silica gel with MeOH/dichloromethane (gradient 0-5% MeOH). All fractions containing product were combined and concentrated. The residue was triturated with dichloromethane/hexanes to afford 38 mg (24%) N-{(S)-1-[(S)-1-((S)-1-benzylaminooxalyl-pentylcarbamoyl)-2-(1H-indol-3-yl)-ethylcarbamoyl]-ethyl}-2,3-dichloro-benzamide as a light yellow solid. LC/MS: (M−H)⁻=676.

### Example 13

### 2-Methyl-2H-pyrazole-3-carboxylic acid {(S)-1-[(S)-1-((S)-1-benzylaminooxalyl-pentylcarbamoyl)-2-(1H-indol-3-yl)-ethylcarbamoyl]-ethyl}-amide

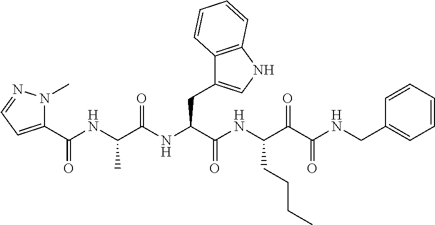 [SMILES: CCCC[C@H](NC(=O)[C@H](Cc1c[nH]c2ccccc12)NC(=O)[C@H](C)NC(=O)c1ccnn1C)C(=O)C(=O)NCc1ccccc1]

In a 50 ml round-bottomed flask, (S)-2-((S)-2-amino-propionylamino)-3-(1H-indol-3-yl)-propionic acid tert-butyl ester (800 mg, 1.93 mmol; purity=80%) was dissolved in 5.9 ml DMF. The pale yellow solution was cooled to 0° C. N,N-Diisopropylethylamine (666 mg, 0.9 ml, 5.15 mmol) was added slowly at 0° C. 1-Methyl-1H-pyrazole-5-carboxylic acid (214 mg, 1.7 mmol) was added followed by HATU (710 mg, 1.87 mmol). After the addition was complete, the ice bath was removed and the reaction mixture was stirred at room temperature overnight. The reaction mixture was extracted with 70 ml diethyl ether and 10 ml water. The aqueous layer was extracted with 70 ml diethyl ether. The organic layers were washed twice with 10 ml water and once with 10 ml brine. The organic layers were combined, dried over sodium sulfate, filtered and concentrated. The residue (light yellow oil) was chromatographed over 40 g silica gel with EtOAc/hexanes (gradient 0-70% EtOAc). All fractions containing product were combined and concentrated to provide 774 mg (99%; purity=95%) (S)-3-(1H-indol-3-yl)-2-{(S)-2-[(2-methyl-2H-pyrazole-3-carbonyl)-amino]-propionylamino}-propionic acid tert-butyl ester as an off-white foam.

A microwave vial was charged with (S)-3-(1H-indol-3-yl)-2-{(S)-2-[(2-methyl-2H-pyrazole-3-carbonyl)-amino]-propionylamino}-propionic acid tert-butyl ester (126 mg, 0.27 mmol) and 1,1,1,3,3,3-hexafluoro-2-propanol (2.2 ml, 20.9 mmol). The vial was flushed with argon and sealed. The colorless solution was heated at 120° C. for 2 h under microwave irradiation (the reaction mixture turned into a light purple solution). The reaction mixture was concentrated to provide (S)-3-(1H-indol-3-yl)-2-{(S)-2-[(2-methyl-2H-pyrazole-3-carbonyl)-amino]-propionylamino}-propionic acid as a purple foam which was used without further purification.

In a 10 ml round-bottomed flask, [(S)-1-(benzylcarbamoyl-hydroxy-methyl)-pentyl]-carbamic acid tert-butyl ester (110 mg, 0.31 mmol) was dissolved in 3.0 ml dichloromethane. Trifluoroacetic acid (0.55 ml, 7.14 mmol) was added slowly. The reaction mixture was stirred at room temperature for 2.5 h. The solvent was evaporated and then placed under high vacuum for 15 min. The residue was dissolved in 1.0 ml DMF and the solution was stirred at 0° C. for 5 min. N,N-Diisopropylethylamine (296 mg, 0.40 ml, 2.29 mmol) was added dropwise at 0° C. (S)-3-(1H-Indol-3-yl)-2-{(S)-2-[(2-methyl-2H-pyrazole-3-carbonyl)-amino]-propionylamino}-propionic acid (169 mg, 0.26 mmol; purity=60%), dissolved in 1.1 ml DMF was added followed by HATU (111 mg, 0.29 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then placed under high vacuum (78 mg off-white solid). The filtrate from above was extracted with 40 ml diethyl ether/EtOAc (3:1). The aqueous layer was extracted with 40 ml diethyl ether/EtOAc (3:1). The organic layers were washed twice with 5 ml water and once with 5 ml brine. The organic layers were combined, dried over sodium sulfate, filtered and concentrated. The residue was triturated with petroleum ether and a few drops of dichloromethane. The resulting off-white solid was redissolved in dichloromethane, combined with the 78 mg off-white solid from above and concentrated to afford 134 mg (82%) 2-methyl-2H-pyrazole-3-carboxylic acid {(S)-1-[(S)-1-[(S)-1-(benzylcarbamoyl-hydroxy-methyl)-pentylcarbamoyl]-2-(1H-indol-3-yl)-ethylcarbamoyl]-ethyl}-amide as an off-white solid and a mixture of epimers.

In a 25 ml round-bottomed flask, 2-methyl-2H-pyrazole-3-carboxylic acid {(S)-1-[(S)-1-[(S)-1-(benzylcarbamoyl-hydroxy-methyl)-pentylcarbamoyl]-2-(1H-indol-3-yl)-ethylcarbamoyl]-ethyl}-amide (131 mg, 0.21 mmol) was partially dissolved in 8.0 ml dichloromethane and Dess-Martin periodinane (135 mg, 0.32 mmol) was added. The reaction mixture was stirred at room temperature for 2.5 h. The reaction mixture was quenched with 3.5 ml saturated NaHCO₃-solution and 3.5 ml 10% Na₂S₂O₃-solution and stirred vigorously for 30 min at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was chromatographed over 12 g silica gel with MeOH/dichloromethane (gradient 0-5% MeOH). All fractions containing product were combined and concentrated. The residue was triturated with dichloromethane/hexanes to afford 29 mg (21%) 2-methyl-2H-pyrazole-3-carboxylic acid {(S)-1-[(S)-1-((S)-1-benzylaminooxalyl-pentylcarbamoyl)-2-(1H-indol-3-yl)-ethylcarbamoyl]-ethyl}-amide as a light brown solid. LC/MS: (M−H)⁻=612.

### Example 14

### (S)-3-{(S)-3-(4-Methoxy-phenyl)-2-[(S)-2-(2-morpholin-4-yl-acetylamino)-propionylamino]-propionylamino}-2-oxo-heptanoic acid methylamide

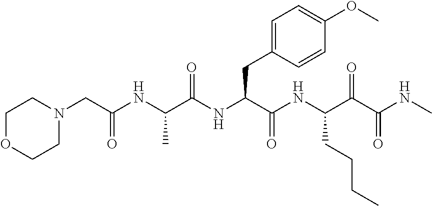 [SMILES: CCCC[C@H](NC(=O)[C@H](Cc1ccc(OC)cc1)NC(=O)[C@H](C)NC(=O)CN1CCOCC1)C(=O)C(=O)NC]

A 10 ml round-bottomed flask was charged with (S)-3-tert-butoxycarbonylamino-2-hydroxy-heptanoic acid (250 mg, 0.96 mmol), methylamine hydrochloride (84 mg, 1.24 mmol) and 4.0 ml dichloromethane. N,N-Diisopropylethylamine (370 mg, 0.50 ml, 2.86 mmol) was added followed by HATU (400 mg, 1.05 mmol). The yellow solution was stirred at room temperature overnight. The reaction mixture was extracted with 3 ml water and 30 ml dichloromethane. The organic layer was washed with 3 ml water. The aqueous layers were extracted twice with 30 ml dichloromethane. The organic layers were combined, dried over sodium sulfate, filtered and concentrated. The residue was chromatographed over 12 g silica gel with EtOAc/hexanes (gradient 0-70% EtOAc). All fractions containing product were combined and concentrated to afford 236 mg (63%; purity=70%) [(S)-1-(hydroxy-methylcarbamoyl-methyl)-pentyl]-carbamic acid tert-butyl ester as a colorless oil and as a mixture of epimers.

In a 10 ml round-bottomed flask, [(S)-1-(hydroxy-methylcarbamoyl-methyl)-pentyl]-carbamic acid tert-butyl ester (231 mg, 0.59 mmol; purity=70%) was dissolved in 4.0 ml dichloromethane. Trifluoroacetic acid (1.0 ml, 13.0 mmol) was added slowly. The reaction mixture was stirred at room temperature for 2.5 h. The solvent was evaporated and then placed under high vacuum for 15 min. The residue (yellow oil) was dissolved in 2.7 ml DMF and the solution was stirred at 0° C. for 5 min. N,N-Diisopropylethylamine (370 mg, 0.50 ml, 2.86 mmol) was added dropwise at 0° C. (S)-3-(4-Methoxy-phenyl)-2-[(S)-2-(2-morpholin-4-yl-acetylamino)-propionylamino]-propionic acid (150 mg, 0.38 mmol) was added followed by HATU (159 mg, 0.42 mmol). After the addition was complete, the ice bath was removed and the reaction mixture was stirred at room temperature overnight. The reaction mixture was extracted with 40 ml diethyl ether/ethyl acetate (1:1) and 3 ml water. The aqueous layer was again extracted with 40 ml diethyl ether/ethyl acetate (1:1). The organic layers were washed twice with 3 ml water and once with 3 ml brine. The organic layers were combined, dried over sodium sulfate, filtered and concentrated. The residue was chromatographed over 12 g silica gel with MeOH/dichloromethane (gradient 0-10% MeOH). All fractions containing product were combined and concentrated to provide 57 mg (27%) (S)-2-hydroxy-3-{(S)-3-(4-methoxy-phenyl)-2-[(S)-2-(2-morpholin-4-yl-acetylamino)-propionylamino]-propionylamino}-heptanoic acid methylamide as an off-white solid and as a mixture of epimers.

In a 25 ml round-bottomed flask, (S)-2-hydroxy-3-{(S)-3-(4-methoxy-phenyl)-2-[(S)-2-(2-morpholin-4-yl-acetylamino)-propionylamino]-propionylamino}-heptanoic acid methylamide (52 mg, 0.09 mmol) was dissolved in 3.5 ml dichloromethane and Dess-Martin periodinane (60 mg, 0.14 mmol) was added. The reaction mixture was stirred at room temperature for 2.5 h. The reaction mixture was quenched with 1.5 ml saturated NaHCO₃-solution and 1.5 ml 10% Na₂S₂O₃-solution and stirred vigorously for 30 min at room temperature. The biphasic mixture was then extracted with 25 ml dichloromethane. The organic layer was washed with 3 ml saturated NaHCO₃-solution. The aqueous layers were extracted twice with 25 ml dichloromethane. The organic layers were combined, dried over sodium sulfate, filtered and concentrated. The residue was absorbed on 1 g silica gel and chromatographed over 4 g silica gel MeOH/dichloromethane (gradient 0-10% MeOH). All fractions containing product were combined and concentrated. The residue was triturated with dichloromethane/hexanes to afford 20 mg (37%) (S)-3-{(S)-3-(4-methoxy-phenyl)-2-[(S)-2-(2-morpholin-4-yl-acetylamino)-propionylamino]-propionylamino}-2-oxo-heptanoic acid methylamide as a white solid. LC/MS: (M+H)⁺=548.

### Example 15

### 3-Methyl-1H-indene-2-carboxylic acid {(S)-1-[(S)-1-((S)-1-benzylaminooxalyl-pentylcarbamoyl)-3-methanesulfonyl-propylcarbamoyl]-ethyl}-amide

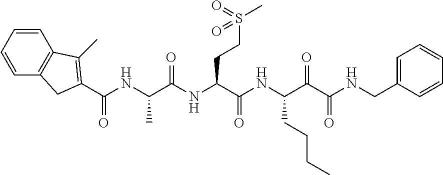 [SMILES: CCCC[C@H](NC(=O)[C@H](CCS(C)(=O)=O)NC(=O)[C@H](C)NC(=O)C1=C(C)c2ccccc2C1)C(=O)C(=O)NCc1ccccc1]

To a solution of (S)-2-(benzyloxycarbonylamino)propanoic acid (900 mg, 4.03 mmol), (S)-tert-butyl 2-amino-4-(methylthio)butanoate hydrochloride (1.02 g, 4.23 mmol), and HATU (1.69 g, 4.43 mmol) in DMF (10 ml) was added N,N-diisopropylethylamine (2.11 ml, 12.1 mmol). Reaction became slightly exothermic so used an ice bath to moderate the temperature. The bright yellow reaction mixture was stirred at room temperature overnight then quenched with water and extracted with EtOAc (2×). The combined organics were washed with water (3×) and brine then dried over MgSO₄ and concentrated to afford 2.1 g of (S)-2-((S)-2-benzyloxycarbonylamino-propionylamino)-4-methylsulfanyl-butyric acid tert-butyl ester as a yellow oil which was used without further purification.

To a solution of (S)-2-((S)-2-benzyloxycarbonylamino-propionylamino)-4-methylsulfanyl-butyric acid tert-butyl ester (1.66 g, 4.04 mmol, crude from Step 1) in CH₂Cl₂ (30 ml) at 0° C. was added m-CPBA (77%, 1.99 g, 8.9 mmol). The cloudy mixture was warmed to room temperature and stirred for 1.5 h then diluted with CH₂Cl₂ (20 ml) and added sat'd aqueous NaHCO₃ (50 ml). The biphasic mixture was stirred vigorously at room temperature for 1 h then the layers were separated and the aqueous layer was extracted with CH₂Cl₂. The combined organics were washed with sat'd NaHCO₃, dried over MgSO₄ and concentrated. The residue was purified by silica gel chromatography (50% to 100% EtOAc/hexanes) to afford 1.48 g (83%) of (S)-2-((S)-2-benzyloxycarbonylamino-propionylamino)-4-methanesulfonyl-butyric acid tert-butyl ester as a white foamy solid. LC/MS: (M+Na)⁺=465.

To a solution of (S)-2-((S)-2-benzyloxycarbonylamino-propionylamino)-4-methanesulfonyl-butyric acid tert-butyl ester (1.48 g, 3.34 mmol) in MeOH (35 ml) was added 20% palladium hydroxide on carbon (200 mg, 0.29 mmol). The reaction mixture was stirred under hydrogen (balloon) for 2 h then filtered over Celite, rinsing with EtOAc/MeOH. To the filtrate was added 1.0 M HCl in MeOH (3.7 ml, 3.7 mmol, freshly prepared from AcCl and MeOH). The filtrate was concentrated to afford 1.27 g of (S)-2-((S)-2-amino-propionylamino)-4-methanesulfonyl-butyric acid tert-butyl ester hydrochloride as a white foamy solid.

To a solution of (S)-2-((S)-2-amino-propionylamino)-4-methanesulfonyl-butyric acid tert-butyl ester hydrochloride (327 mg, 0.95 mmol), 3-methyl-1H-indene-2-carboxylic acid (150 mg, 0.86 mmol), and HATU (360 mg, 0.95 mmol) in DMF (4 ml) at 0° C. was added N,N-diisopropylethylamine (45 ml, 2.58 mmol). The reaction mixture was stirred at room temperature overnight then quenched with water and extracted with EtOAc (2×). The combined organics were washed with water (3×) dried over MgSO₄ and concentrated. The residue was purified by silica gel chromatography (50% to 100% EtOAc/hexanes) to isolate 336 mg (84%) of (S)-4-methanesulfonyl-2-{(S)-2-[(3-methyl-1H-indene-2-carbonyl)-amino]-propionylamino}-butyric acid tert-butyl ester as a white solid. LC/MS: (M+H)⁺=465.

To a solution of [(S)-1-(benzylcarbamoyl-hydroxy-methyl)-pentyl]-carbamic acid tert-butyl ester (98 mg, 0.28 mmol) in CH₂Cl₂ (3 ml) was added trifluoroacetic acid (0.5 ml). The reaction mixture was stirred at room temperature for 2 h then concentrated and chased with hexanes to provide a colorless oil. To a solution of (S)-4-methanesulfonyl-2-{(S)-2-[(3-methyl-1H-indene-2-carbonyl)-amino]-propionylamino}-butyric acid tert-butyl ester (100 mg, 0.22 mmol) in CH₂Cl₂ (3 ml) was added trifluoroacetic acid (0.5 ml). The reaction mixture was stirred at room temperature for 2 h then concentrated and chased with hexanes to provide a pink oil. The two oils from above were dissolved in CH₂Cl₂, combined, concentrated and dried under high vacuum. The residue was dissolved in DMF (3 ml) and HATU (90 mg, 0.24 mmol) was added. The reaction mixture was cooled to 0° C. and N,N-diisopropylethylamine (0.30 ml, 1.72 mmol) was added. The reaction mixture was warmed to room temperature and stirred overnight then quenched with water. The resultant precipitate was collected via filtration, rinsed with water and dried under high vacuum to afford 120 mg (87%) of 3-methyl-1H-indene-2-carboxylic acid ((S)-1-{(S)-1-[(S)-1-(benzylcarbamoyl-hydroxy-methyl)-pentylcarbamoyl]-3-methanesulfonyl-propylcarbamoyl}-ethyl)-amide as a light yellow solid and a mixture of epimers. LC/MS: (M+Na)⁺=663.

A partial suspension of 3-methyl-1H-indene-2-carboxylic acid ((S)-1-{(S)-1-[(S)-1-(benzylcarbamoyl-hydroxy-methyl)-pentylcarbamoyl]-3-methanesulfonyl-propylcarbamoyl}-ethyl)-amide (120 mg, 0.187 mmol) in CH₂Cl₂ (8 ml) and THF (1 ml) was sonicated until most solids dissolved. Then Dess-Martin periodinane (119 mg, 0.28 mmol) was added and the cloudy mixture was stirred at room temperature for 1.5 h. A thick white precipitate had formed. Added 10% aqueous Na₂S₂O₃ (5 ml) and sat'd NaHCO₃ (5 mL) and the biphasic mixture was stirred vigorously for 30 min then diluted with water and extracted with CH₂Cl₂ (2×). The combined organics were washed with sat'd NaHCO₃ and concentrated. The residue was absorbed on silica gel and purified by chromatography (0% to 5% MeOH/CH₂Cl₂) to afford 48 mg (40%) of 3-methyl-1H-indene-2-carboxylic acid {(S)-1-[(S)-1-((S)-1-benzylaminooxalyl-pentylcarbamoyl)-3-methanesulfonyl-propylcarbamoyl]-ethyl}-amide as a pale yellow solid. LC/MS: (M+H)⁺=639.

### Example 16

### {(S)-1-[(S)-1-((S)-1-Benzyl-2-benzylcarbamoyl-2-oxo-ethylcarbamoyl)-2-(1H-indol-3-yl)-ethylcarbamoyl]-ethyl}-carbamic acid benzyl ester

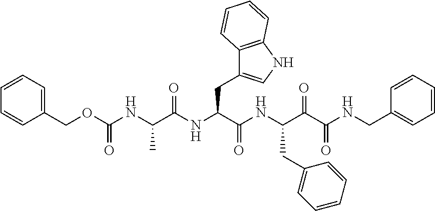 [SMILES: C[C@H](NC(=O)OCc1ccccc1)C(=O)N[C@@H](Cc1c[nH]c2ccccc12)C(=O)N[C@@H](Cc1ccccc1)C(=O)C(=O)NCc1ccccc1]

In a 10 ml round-bottomed flask, ((S)-1-benzyl-2-benzylcarbamoyl-2-hydroxy-ethyl)-carbamic acid tert-butyl ester (125 mg, 0.33 mmol) was dissolved in 3.4 ml dichloromethane. Trifluoroacetic acid (0.57 ml, 7.4 mmol) was added slowly. The reaction mixture was stirred at room temperature for 3 h. The solvent was evaporated and then put under high vacuum for 30 min. The residue (yellow oil) was dissolved in 1.0 ml DMF and the solution was stirred at 0° C. for 5 min. N,N-Diisopropylethylamine (222 mg, 0.30 ml, 1.72 mmol) was added dropwise at 0° C. (S)-2-((S)-2-Benzyloxycarbonylamino-propionylamino)-3-(1H-indol-3-yl)-propionic acid (158 mg, 0.25 mmol; purity=65%), dissolved in 1.2 ml DMF was added followed by HATU (105 mg, 0.28 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then placed under high vacuum to provide 118 mg (70%) {(S)-1-[(S)-1-((S)-1-benzyl-2-benzylcarbamoyl-2-hydroxy-ethylcarbamoyl)-2-(1H-indol-3-yl)-ethylcarbamoyl]-ethyl}-carbamic acid benzyl ester as a mixture of epimers.

In a 25 ml round-bottomed flask, {(S)-1-[(S)-1-((S)-1-benzyl-2-benzylcarbamoyl-2-hydroxy-ethylcarbamoyl)-2-(1H-indol-3-yl)-ethylcarbamoyl]-ethyl}-carbamic acid benzyl ester (117 mg, 0.17 mmol) was dissolved in 6.5 ml dichloromethane and Dess-Martin periodinane (110 mg, 0.26 mmol) was added. The reaction mixture was stirred at room temperature for 2.5 h. The reaction mixture was quenched with 3 ml saturated NaHCO₃-solution and 3 ml 10% Na₂S₂O₃-solution and stirred vigorously for 30 min at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was chromatographed over 12 g silica gel with MeOH/dichloromethane (gradient 0-5% MeOH). All fractions containing product were combined and concentrated. The residue was triturated with dichloromethane/hexanes to afford 33 mg (27%) {(S)-1-[(S)-1-((S)-1-benzyl-2-benzylcarbamoyl-2-oxo-ethylcarbamoyl)-2-(1H-indol-3-yl)-ethylcarbamoyl]-ethyl}-carbamic acid benzyl ester as a light brown solid. LC/MS: (M+H)⁺=674.

### Example 17

### {(S)-1-[(S)-1-((S)-1-Benzyl-2-benzylcarbamoyl-2-oxo-ethylcarbamoyl)-2-(4-methoxy-phenyl)-ethylcarbamoyl]-ethyl}-carbamic acid benzyl ester

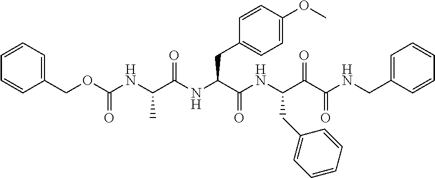 [SMILES: COc1ccc(C[C@H](NC(=O)[C@H](C)NC(=O)OCc2ccccc2)C(=O)N[C@@H](Cc2ccccc2)C(=O)C(=O)NCc2ccccc2)cc1]

To a solution of (S)-2-tert-butoxycarbonylamino-3-(4-methoxy-phenyl)-propionic acid (1.0 g, 3.39 mmol) in MeOH (20 ml) at 0° C. was added dropwise trimethylsilyldiazomethane (2.0 M in Et₂O, 3.4 ml, 6.8 mmol). Additional trimethylsilyldiazomethane (2.0 M in Et₂O) was added in 1 mL aliquots until a pale yellow color persisted. A total of 10 mL (˜6 eq) of reagent was added. The reaction was quenched with a few drops of acetic acid where upon solution became colorless. The mixture was concentrated to afford 1.13 g of (S)-2-tert-butoxycarbonylamino-3-(4-methoxy-phenyl)-propionic acid methyl ester as a colorless oil which was used without further purification. ¹H NMR (CDCl₃) δ: 6.88 (d, J=8.3 Hz, 1H), 6.63-6.72 (m, 1H), 4.80 (d, J=7.9 Hz, 1H), 4.33-4.45 (m, 1H), 3.63 (s, 3H), 3.56 (s, 3H), 2.87 (t, J=5.9 Hz, 2H), 1.26 (s, 9H).

To a solution of (S)-2-tert-butoxycarbonylamino-3-(4-methoxy-phenyl)-propionic acid methyl ester (1.05 g, 3.39 mmol) in CH₂Cl₂ (12 ml) was added TFA (3 ml). The reaction mixture was stirred room temperature for 3 h then concentrated, chased with hexanes (2×) and dried under high vacuum. The residue was dissolved in DMF (8 ml) and cooled to 0° C. Then added (S)-2-(benzyloxycarbonylamino)propanoic acid (660 mg, 2.96 mmol) and HATU (1.24 g, 3.25 mmol) followed by N,N-diisopropylethylamine (2.6 ml, 14.8 mmol). The reaction mixture was warmed to room temperature and stirred overnight then quenched with water and extracted with EtOAc (2×). The combined organics were washed with water (3×) and brine then dried over MgSO₄ and concentrated. The residue was purified by silica gel chromatography (20% to 50% EtOAc/hexanes) to afford 994 mg (81%) of (S)-2-((S)-2-benzyloxycarbonylamino-propionylamino)-3-(4-methoxy-phenyl)-propionic acid methyl ester as a white solid. LC/MS: (M+H)⁺=415.

To a solution of (S)-2-((S)-2-benzyloxycarbonylamino-propionylamino)-3-(4-methoxy-phenyl)-propionic acid methyl ester (250 mg, 0.60 mmol) in 1,2-dichloroethane (6 ml) was added trimethyltin hydroxide (327 mg, 1.81 mmol). The reaction mixture was heated at 80° C. for 3 h. Additional trimethyltin hydroxide (100 mg, 0.55 mmol) was added and heating was continued for 2 h. The reaction was cooled to room temperature and concentrated under reduced pressure. The residue was dissolved in EtOAc and washed with 0.5 M aqueous HCl (3×). The organic phase was dried over MgSO₄ and concentrated to afford 330 mg of (S)-2-((S)-2-benzyloxycarbonylamino-propionylamino)-3-(4-methoxy-phenyl)-propionic acid as a white solid which was used without further purification.

To a solution of ((S)-1-benzyl-2-benzylcarbamoyl-2-hydroxy-ethyl)-carbamic acid tert-butyl ester (100 mg, 0.26 mmol) in CH₂Cl₂ (4 ml) was added trifluoroacetic acid (0.5 ml). The reaction mixture was stirred at room temperature for 2 h then concentrated and chased with hexanes. The residue was dissolved in DMF (3 ml) and (S)-2-((S)-2-benzyloxycarbonylamino-propionylamino)-3-(4-methoxy-phenyl)-propionic acid (80 mg, 0.22 mmol) and HATU (90 mg, 0.24 mmol) were added. The mixture was cooled to 0° C. and N,N-diisopropylethylamine (0.30 ml, 1.73 mmol) was added. The yellow mixture was warmed to room temperature and stirred overnight then quenched with water. The resultant precipitate was collected via filtration, rinsed with water, and dried under high vacuum to afford 112 mg (78%) of {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-carbamic acid benzyl ester as an off-white solid and a mixture of epimers. LC/MS: (M-OBn)⁻=557.

To a partial suspension of {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-carbamic acid benzyl ester (106 mg, 0.16 mmol) in CH₂Cl₂ (10 ml) was added Dess-Martin periodinane (101 mg, 0.24 mmol). After a few minutes most solids had dissolved. The slightly cloudy mixture was stirred at room temperature for 2 h during which time a thick white precipitate was formed. Added 10% aqueous Na₂S₂O₃ (5 ml) and sat'd NaHCO₃ (5 mL). The biphasic mixture was stirred vigorously for 20 min then diluted with water and extracted with CH₂Cl₂ (2×). The combined organics were washed with sat'd NaHCO3 and concentrated. The residue was absorbed on silica gel and purified by chromatography (0% to 5% MeOH/CH₂Cl₂) to isolate an off-white solid. Triturated with CH₂Cl₂/hexanes to afford 52 mg (49%) of {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-carbamic acid benzyl ester as a white solid. LC/MS: (M+Na)⁺=687.

### Example 18

### Indan-2-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide

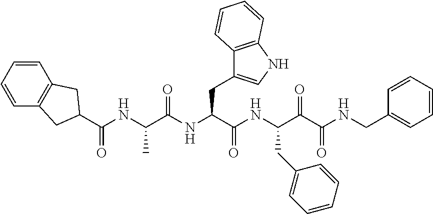 [SMILES: C[C@H](NC(=O)C1Cc2ccccc2C1)C(=O)N[C@@H](Cc1c[nH]c2ccccc12)C(=O)N[C@@H](Cc1ccccc1)C(=O)C(=O)NCc1ccccc1]

In a 10 ml round-bottomed flask, ((S)-1-Benzyl-2-benzylcarbamoyl-2-hydroxy-ethyl)-carbamic acid tert-butyl ester (130 mg, 0.34 mmol) was dissolved in 4.0 ml dichloromethane. Trifluoroacetic acid (0.70 ml, 9.09 mmol) was added slowly. The reaction mixture was stirred at room temperature for 2.5 h. The solvent was evaporated and then put under high vacuum for 30 min. The residue was dissolved in 1.2 ml DMF and the solution was stirred at 0° C. for 5 min. N,N-Diisopropylethylamine (244 mg, 0.33 ml, 1.89 mmol) was added dropwise at 0° C. (S)-2-{(S)-2-[(Indane-2-carbonyl)-amino]-propionylamino}-3-(1H-indol-3-yl)-propionic acid (253 mg, 0.27 mmol; purity=45%), dissolved in 1.5 ml DMF was added followed by HATU (114 mg, 0.30 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then placed under high vacuum to provide 185 mg (94%) indan-2-carboxylic acid {(S)-1-[(S)-1-((S)-1-benzyl-2-benzylcarbamoyl-2-hydroxy-ethylcarbamoyl)-2-(1H-indol-3-yl)-ethylcarbamoyl]-ethyl}-amide as a mixture of epimers.

In a 25 ml round-bottomed flask, indan-2-carboxylic acid {(S)-1-[(S)-1-((S)-1-benzyl-2-benzylcarbamoyl-2-hydroxy-ethylcarbamoyl)-2-(1H-indol-3-yl)-ethylcarbamoyl]-ethyl}-amide (182 mg, 0.25 mmol) was dissolved in 9.5 ml dichloromethane and Dess-Martin periodinane (160 mg, 0.38 mmol) was added. The reaction mixture was stirred at room temperature for 2.5 h. The reaction mixture was quenched with 4 ml saturated NaHCO₃-solution and 4 ml 10% Na₂S₂O₃-solution and stirred vigorously for 45 min at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue was absorbed on 1 g silica gel and rechromatographed over 12 g silica gel with MeOH/dichloromethane (gradient 0-5% MeOH). All fractions containing product were combined and concentrated to afford 20 mg (11%) indan-2-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide as a light brown solid. LC/MS: (M−H)⁻=682.

### Example 19

### Indan-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide

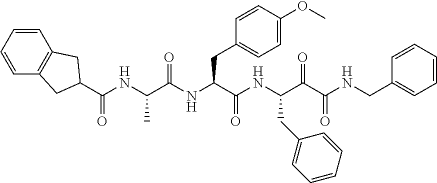 [SMILES: COc1ccc(C[C@H](NC(=O)[C@H](C)NC(=O)C2Cc3ccccc3C2)C(=O)N[C@@H](Cc2ccccc2)C(=O)C(=O)NCc2ccccc2)cc1]

In a 50 ml round-bottomed flask, (S)-2-((S)-2-tert-butoxycarbonylamino-propionylamino)-3-(4-methoxy-phenyl)-propionic acid benzyl ester (762 mg, 1.5 mmol; purity=90%) was dissolved in 8.0 ml dichloromethane. Trifluoroacetic acid (2.7 ml, 35.0 mmol) was added slowly. The reaction mixture was stirred at room temperature for 2.5 h. The solvent was evaporated and then placed under high vacuum for 30 min. The residue (pale brown oil) was dissolved in 5.0 ml DMF and the solution was stirred at 0° C. for 10 min. N,N-Diisopropylethylamine (1.11 g, 1.5 ml, 8.59 mmol) was added dropwise at 0° C. 2-Indanecarboxylic acid (225 mg, 1.39 mmol) was added followed by HATU (580 mg, 1.53 mmol). After the addition was complete, the ice bath was removed and the reaction mixture was stirred at room temperature for 48 h. The reaction mixture was diluted with water and petroleum ether (an off-white precipitate was formed). The suspension was filtered, rinsing with water and a little petroleum ether. The resulting off-white solid was dried using the rotavap and then placed under high vacuum to provide 614 mg (88%) (S)-2-{(S)-2-[(indane-2-carbonyl)-amino]-propionylamino}-3-(4-methoxy-phenyl)-propionic acid benzyl ester as an off-white solid.

In a 100 ml round-bottomed flask, (S)-2-{(S)-2-[(indane-2-carbonyl)-amino]-propionylamino}-3-(4-methoxy-phenyl)-propionic acid benzyl ester (612 mg, 1.22 mmol) was dissolved in 15 ml methanol and 15 ml THF. The flask was three times alternating evacuated and flushed with argon. 20% Palladium hydroxide on carbon (wet, 111 mg, 0.16 mmol) was added carefully. The flask was evacuated, flushed with argon, evacuated and flushed with hydrogen. The reaction mixture was stirred under hydrogen atmosphere (balloon) at room temperature for 2 h. The reaction mixture was filtered over Celite, rinsing with ethyl acetate/methanol. The filtrate was concentrated to afford 518 mg (98%; purity=95%) (S)-2-{(S)-2-[(indane-2-carbonyl)-amino]-propionylamino}-3-(4-methoxy-phenyl)-propionic acid as an off-white solid.

In a 10 ml round-bottomed flask, ((S)-1-Benzyl-2-benzylcarbamoyl-2-hydroxy-ethyl)-carbamic acid tert-butyl ester (120 mg, 0.31 mmol) was dissolved in 3.2 ml dichloromethane. Trifluoroacetic acid (0.56 ml, 7.27 mmol) was added slowly. The reaction mixture was stirred at room temperature for 2.5 h. The solvent was evaporated and then put under high vacuum for 30 min. The residue was dissolved in 2.2 ml DMF and the solution was stirred at 0° C. for 5 min. N,N-Diisopropylethylamine (244 mg, 0.33 ml, 1.89 mmol) was added dropwise at 0° C. (S)-2-{(S)-2-[(Indane-2-carbonyl)-amino]-propionylamino}-3-(4-methoxy-phenyl)-propionic acid (120 mg, 0.28 mmol) was added followed by HATU (116 mg, 0.31 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then placed under high vacuum to provide 186 mg (99%) indan-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide as a mixture of epimers.

In a 25 ml round-bottomed flask, indan-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide (174 mg, 0.26 mmol) was dissolved in 9.5 ml dichloromethane and Dess-Martin periodinane (164 mg, 0.39 mmol) was added. The reaction mixture was stirred at room temperature for 2.5 h. The reaction mixture was quenched with 4 ml saturated NaHCO₃-solution and 4 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue was triturated with dichloromethane/hexanes to afford 42 mg (23%) indan-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide as a white solid. LC/MS: (M−H)⁻=673.

### Example 20

### 3-Methyl-1H-indene-2-carboxylic acid {(S)-1-[(S)-1-((S)-1-benzyl-2-benzylcarbamoyl-2-oxo-ethylcarbamoyl)-3-methanesulfonyl-propylcarbamoyl]-ethyl}-amide

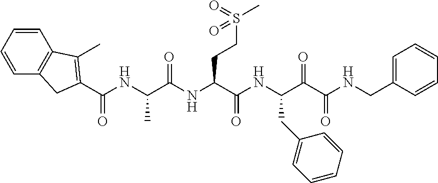 [SMILES: CC1=C(C(=O)N[C@@H](C)C(=O)N[C@@H](CCS(C)(=O)=O)C(=O)N[C@@H](Cc2ccccc2)C(=O)C(=O)NCc2ccccc2)Cc2ccccc21]

To a solution of (S)-4-methanesulfonyl-2-{(S)-2-[(3-methyl-1H-indene-2-carbonyl)-amino]-propionylamino}-butyric acid tert-butyl ester (100 mg, 0.22 mmol) and ((S)-1-benzyl-2-benzylcarbamoyl-2-hydroxy-ethyl)-carbamic acid tert-butyl ester (100 mg, 0.26 mmol) in CH₂Cl₂ (4 ml) was added trifluoroacetic acid (1.0 ml). The reaction mixture was stirred at room temperature for 2 h then concentrated and chased with hexanes. The residue was dissolved in DMF (3 ml) and HATU (90 mg, 0.24 mmol) was added. The reaction mixture was cooled to 0° C. and N,N-diisopropylethylamine (0.30 ml, 1.72 mmol) was added. The reaction mixture was warmed to room temperature and stirred overnight then quenched with water. The resultant precipitate was collected via filtration and rinsed with water and Et₂O/petroleum ether then dried under high vacuum to afford 133 mg (92%) of 3-methyl-1H-indene-2-carboxylic acid {(S)-1-[(S)-1-((S)-1-benzyl-2-benzylcarbamoyl-2-hydroxy-ethylcarbamoyl)-3-methanesulfonyl-propylcarbamoyl]-ethyl}-amide as an off-white solid and a mixture of epimers. (M−H)⁻=673.

A partial suspension of 3-methyl-1H-indene-2-carboxylic acid {(S)-1-[(S)-1-((S)-1-benzyl-2-benzylcarbamoyl-2-hydroxy-ethylcarbamoyl)-3-methanesulfonyl-propylcarbamoyl]-ethyl}-amide (130 mg, 0.19 mmol) in CH₂Cl₂ (8 ml) and THF (4 ml) was sonicated until most solids dissolved. Then Dess-Martin periodinane (123 mg, 0.29 mmol) was added and the cloudy mixture was stirred at room temperature for 1.5 h. A thick white precipitate had formed. Added 10% aqueous Na₂S₂O₃ (5 ml) and sat'd NaHCO₃ (5 mL) and the biphasic mixture was stirred vigorously for 10 min then diluted with water and extracted with 5% MeOH/CH₂Cl₂ (2×). The combined organics were washed with sat'd NaHCO₃ and concentrated. The residue was absorbed on silica gel and purified by chromatography (0% to 5% MeOH/CH₂Cl₂) follow by trituration with CH₂Cl₂/hexanes to afford 43 mg (33%) of 3-methyl-1H-indene-2-carboxylic acid {(S)-1-[(S)-1-((S)-1-benzyl-2-benzylcarbamoyl-2-oxo-ethylcarbamoyl)-3-methanesulfonyl-propylcarbamoyl]-ethyl}-amide as an off-white solid. LC/MS: (M+H)⁺=673.

### Example 21

### 5-Methyl-isoxazole-3-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide

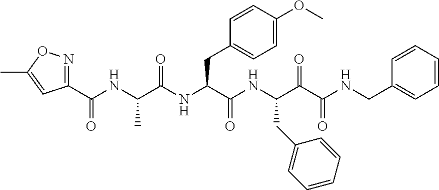 [SMILES: COc1ccc(C[C@H](NC(=O)[C@H](C)NC(=O)c2cc(C)on2)C(=O)N[C@@H](Cc2ccccc2)C(=O)C(=O)NCc2ccccc2)cc1]

In a 50 ml round-bottomed flask, (S)-2-((S)-2-tert-butoxycarbonylamino-propionylamino)-3-(4-methoxy-phenyl)-propionic acid benzyl ester (740 mg, 1.46 mmol; purity=90%) was dissolved in 8.0 ml dichloromethane. Trifluoroacetic acid (2.6 ml, 33.7 mmol) was added slowly. The reaction mixture was stirred at room temperature for 3 h. The solvent was evaporated and then placed under high vacuum for 30 min. The residue (pale brown oil) was dissolved in 5.0 ml DMF and the solution was stirred at 0° C. for 10 min. N,N-Diisopropylethylamine (1.11 g, 1.5 ml, 8.59 mmol) was added dropwise at 0° C. 5-Methylisoxazole-3-carboxylic acid (170 mg, 1.34 mmol) was added followed by HATU (559 mg, 1.47 mmol). After the addition was complete, the ice bath was removed and the reaction mixture was stirred at room temperature for 48 h. The reaction mixture was extracted with 70 ml diethyl ether and 5 ml water. The aqueous layer was extracted with 70 ml diethyl ether. The organic layers were washed three times with 5 ml water and once with 5 ml brine. The organic layers were combined, dried over sodium sulfate, filtered and concentrated to provide (S)-3-(4-methoxy-phenyl)-2-{(S)-2-[(5-methyl-isoxazole-3-carbonyl)-amino]-propionylamino}-propionic acid benzyl ester as a light brown oil which was used without further purification.

In a 100 ml round-bottomed flask, (S)-3-(4-Methoxy-phenyl)-2-{(S)-2-[(5-methyl-isoxazole-3-carbonyl)-amino]-propionylamino}-propionic acid benzyl ester (714 mg, 1.3 mmol; purity=85%) was dissolved in 13 ml methanol. The flask was three times alternating evacuated and flushed with argon. 20% Palladium hydroxide on carbon (wet, 118 mg, 0.17 mmol) was added carefully. The flask was evacuated, flushed with argon, evacuated and flushed with hydrogen. The reaction mixture was stirred under hydrogen atmosphere (balloon) at room temperature for 2 h. The reaction mixture was filtered over Celite, rinsing with ethyl acetate/methanol. The filtrate was concentrated to afford (S)-3-(4-methoxy-phenyl)-2-{(S)-2-[(5-methyl-isoxazole-3-carbonyl)-amino]-propionylamino}-propionic acid as an off-white foam which was used without further purification.

In a 10 ml round-bottomed flask, ((S)-1-Benzyl-2-benzylcarbamoyl-2-hydroxy-ethyl)-carbamic acid tert-butyl ester (120 mg, 0.31 mmol) was dissolved in 3.2 ml dichloromethane. Trifluoroacetic acid (0.56 ml, 7.27 mmol) was added slowly. The reaction mixture was stirred at room temperature for 2.5 h. The solvent was evaporated and then placed under high vacuum for 15 min. The residue was dissolved in 2.2 ml DMF and the solution was stirred at 0° C. for 5 min. N,N-Diisopropylethylamine (244 mg, 0.33 ml, 1.89 mmol) was added dropwise at 0° C. (S)-3-(4-Methoxy-phenyl)-2-{(S)-2-[(5-methyl-isoxazole-3-carbonyl)-amino]-propionylamino}-prop ionic acid (120 mg, 0.27 mmol; purity=85%) was added followed by HATU (114 mg, 0.30 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The resulting off-white solid was dried using the rotavap and then placed under high vacuum to afford 149 mg (85%) 5-methyl-isoxazole-3-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide as a mixture of epimers.

In a 25 ml round-bottomed flask, 5-methyl-isoxazole-3-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide (145 mg, 0.23 mmol) was suspended (partially dissolved) in 8.5 ml dichloromethane and Dess-Martin periodinane (144 mg, 0.34 mmol) was added. The reaction mixture was stirred at room temperature for 3.5 h (a thick precipitate formed). The reaction mixture was quenched with 3.5 ml saturated NaHCO₃-solution and 3.5 ml 10% Na₂S₂O₃-solution and stirred vigorously for 30 min at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue was triturated with dichloromethane/diethyl ether/hexanes to afford 24 mg (15%; purity=90%) 5-methyl-isoxazole-3-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide as an off-white solid. LC/MS: (M−H)⁻=638.

### Example 22

### (S)—N-Benzyl-3-{(S)-3-(1H-indol-3-yl)-2-[(S)-2-(2-morpholin-4-yl-acetylamino)-propionylamino]-propionylamino}-2-oxo-4-phenyl-butyramide

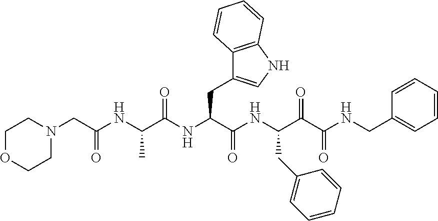 [SMILES: C[C@H](NC(=O)CN1CCOCC1)C(=O)N[C@@H](Cc1c[nH]c2ccccc12)C(=O)N[C@@H](Cc1ccccc1)C(=O)C(=O)NCc1ccccc1]

In a 10 ml round-bottomed flask, ((S)-1-Benzyl-2-benzylcarbamoyl-2-hydroxy-ethyl)-carbamic acid tert-butyl ester (150 mg, 0.39 mmol) was dissolved in 4.0 ml dichloromethane. Trifluoroacetic acid (0.67 ml, 8.7 mmol) was added slowly. The reaction mixture was stirred at room temperature for 2.5 h. The solvent was evaporated and then placed under high vacuum for 15 min. The residue and (S)-3-(1H-indol-3-yl)-2-[(S)-2-(2-morpholin-4-yl-acetylamino)-propionylamino]-propionic acid (270 mg, 0.34 mmol; purity=50%) were dissolved in 2.7 ml dichloromethane and cooled to 0° C. N,N-Diisopropylethylamine (296 mg, 0.40 ml, 2.29 mmol) was added dropwise at 0° C. followed by HATU (140 mg, 0.37 mmol). After the addition was complete, the ice bath was removed and the reaction mixture was stirred at room temperature overnight. The reaction mixture was extracted with 5 ml water and 40 ml dichloromethane. The organic layer was washed with 5 ml water. The aqueous layers were extracted twice with 40 ml dichloromethane. The organic layers were combined, dried over sodium sulfate, filtered and concentrated. The residue was chromatographed over 12 g silica gel with MeOH/dichloromethane (gradient 0-10% MeOH). All fractions containing product were combined and concentrated to provide 161 mg (72%) (S)—N-benzyl-2-hydroxy-3-{(S)-3-(1H-indol-3-yl)-2-[(S)-2-(2-morpholin-4-yl-acetylamino)-propionylamino]-propionylamino}-4-phenyl-butyramide as a light yellow solid and as a mixture of epimers.

In a 25 ml round-bottomed flask, (S)—N-benzyl-2-hydroxy-3-{(S)-3-(1H-indol-3-yl)-2-[(S)-2-(2-morpholin-4-yl-acetylamino)-propionylamino]-propionylamino}-4-phenyl-butyramide (156 mg, 0.23 mmol) was dissolved in 8.5 ml dichloromethane and Dess-Martin periodinane (148 mg, 0.35 mmol) was added. The reaction mixture was stirred at room temperature for 2.5 h. The reaction mixture was quenched with 3.5 ml saturated NaHCO₃-solution and 3.5 ml 10% Na₂S₂O₃-solution and stirred vigorously for 1 h at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue was triturated with dichloromethane/hexanes to afford 13 mg (8%) (S)—N-benzyl-3-{(S)-3-(1H-indol-3-yl)-2-[(S)-2-(2-morpholin-4-yl-acetylamino)-propionylamino]-propionylamino}-2-oxo-4-phenyl-butyramide as an off-white solid. LC/MS: (M+H)⁺=667.

### Example 23

### Indan-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-2-methoxy-ethyl}-amide

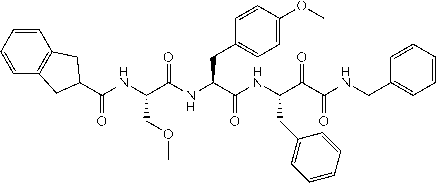 [SMILES: COC[C@H](NC(=O)C1Cc2ccccc2C1)C(=O)N[C@@H](Cc1ccc(OC)cc1)C(=O)N[C@@H](Cc1ccccc1)C(=O)C(=O)NCc1ccccc1]

Boc-O-methyl-L-serine dicyclohexylammonium salt (1.300 g, 3.25 mmol) was dissolved in 50 ml dichloromethane and extracted with 10 ml 1M HCl. The organic layer was washed with 10 ml 1M HCl. The aqueous layers were extracted twice with 50 ml Dichloromethane and then twice with EtOAc. The organic layers were combined, dried over sodium sulfate, filtered and concentrated to afford Boc-O-methyl-L-serine which was used without further purification. In a 50 ml round-bottomed flask, (S)-2-tert-butoxycarbonylamino-3-(4-methoxy-phenyl)-propionic acid benzyl ester (1.340 g, 3.3 mmol) was dissolved in 10.5 ml dichloromethane. Trifluoroacetic acid (7.0 ml, 90.9 mmol) was added slowly. The reaction mixture was stirred at room temperature for 3.5 h. The solvent was evaporated and then placed under high vacuum for 30 min. The residue (pale brown oil) was dissolved in 5.0 ml DMF and the solution was stirred at 0° C. for 10 min. N,N-Diisopropylethylamine (3.18 g, 4.3 ml, 24.6 mmol) was added dropwise at 0° C. Boc-O-methyl-L-serine dissolved in 3.0 ml DMF was added followed by HATU (1.36 g, 3.57 mmol). After the addition was complete, the ice bath was removed and the reaction mixture was stirred at room temperature for 2 d. The reaction mixture was extracted with 80 ml diethyl ether and 10 ml water. The aqueous layer was extracted with 80 ml diethyl ether. The organic layers were washed twice with 10 ml water and once with 10 ml brine. The organic layers were combined, dried over sodium sulfate, filtered and concentrated to afford (S)-2-((S)-2-tert-butoxycarbonylamino-3-methoxy-propionylamino)-3-(4-methoxy-phenyl)-propionic acid benzyl ester as a light brown oil which was used without further purification.

In a 100 ml round-bottomed flask, (S)-2-((S)-2-tert-butoxycarbonylamino-3-methoxy-propionylamino)-3-(4-methoxy-phenyl)-propionic acid benzyl ester (2.093 g, 3.01 mmol; purity=70%) was dissolved in 9.0 ml dichloromethane. Trifluoroacetic acid (6.0 ml, 77.9 mmol) was added slowly. The reaction mixture was stirred at room temperature for 2.5 h. The solvent was evaporated and then placed under high vacuum for 15 min. The residue (light brown solid) was dissolved in 6.0 ml DMF and the solution was stirred at 0° C. for 10 min. N,N-Diisopropylethylamine (2.29 g, 3.1 ml, 17.7 mmol) was added dropwise at 0° C. 2-Indanecarboxylic acid (444 mg, 2.74 mmol) was added followed by HATU (1.15 g, 3.01 mmol). After the addition was complete, the ice bath was removed and the reaction mixture was stirred at room temperature overnight. The reaction mixture was extracted with 80 ml diethyl ether and 10 ml water. The aqueous layer was extracted with 80 ml diethyl ether. The organic layers were washed twice with 10 ml water and once with 10 ml brine. The organic layers were combined, dried over sodium sulfate, filtered and concentrated to afford 1.255 g (86%) (S)-2-{(S)-2-[(indane-2-carbonyl)-amino]-3-methoxy-propionylamino}-3-(4-methoxy-phenyl)-propionic acid benzyl ester as an off-white solid.

In a 150 ml round-bottomed flask, (S)-2-{(S)-2-[(indane-2-carbonyl)-amino]-3-methoxy-propionylamino}-3-(4-methoxy-phenyl)-propionic acid benzyl ester (1.250 g, 2.36 mmol) was dissolved in 12 ml methanol and 12 ml THF. The flask was three times alternating evacuated and flushed with argon. 20% Palladium hydroxide on carbon (213 mg, 0.30 mmol) was added carefully. The flask was evacuated, flushed with argon, evacuated and flushed with hydrogen. The reaction mixture was stirred under hydrogen atmosphere (balloon) at room temperature for 2.5 h. The reaction mixture was filtered over Celite, rinsing with ethyl acetate/methanol. The filtrate was concentrated to afford 1.024 g (99%) (S)-2-{(S)-2-[(indane-2-carbonyl)-amino]-3-methoxy-propionylamino}-3-(4-methoxy-phenyl)-propionic acid as an off-white solid.

In a 10 ml round-bottomed flask, ((S)-1-benzyl-2-benzylcarbamoyl-2-hydroxy-ethyl)-carbamic acid tert-butyl ester (120 mg, 0.31 mmol) was dissolved in 3.2 ml dichloromethane. Trifluoroacetic acid (0.56 ml, 7.27 mmol) was added slowly. The reaction mixture was stirred at room temperature for 3 h. The solvent was evaporated and then placed under high vacuum for 15 min. The residue was dissolved in 2.2 ml DMF and the solution was stirred at 0° C. for 5 min. N,N-Diisopropylethylamine (259 mg, 0.35 ml, 2.00 mmol) was added dropwise at 0° C. (S)-2-{(S)-2-[(Indane-2-carbonyl)-amino]-3-methoxy-propionylamino}-3-(4-methoxy-phenyl)-propionic acid (130 mg, 0.30 mmol) was added followed by HATU (123 mg, 0.32 mmol). After the addition was complete, the ice bath was removed and the reaction mixture was stirred at room temperature overnight. The reaction mixture was diluted with water and petroleum ether (an off-white precipitate was formed). The suspension was filtered, rinsing with water and a little petroleum ether. The residue was absorbed on 1 g silica gel and chromatographed over 12 g silica gel with MeOH/dichloromethane (gradient 0-5% MeOH). All fractions containing product were combined and concentrated to afford 123 mg (59%) indan-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-2-methoxy-ethyl}-amide as an off-white solid and as a mixture of epimers.

In a 25 ml round-bottomed flask, indan-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-2-methoxy-ethyl}-amide (122 mg, 0.17 mmol) was dissolved in 8.5 ml dichloromethane and Dess-Martin periodinane (110 mg, 0.26 mmol) was added. The reaction mixture was stirred at room temperature for 2 h. The reaction mixture was quenched with 3 ml saturated NaHCO₃-solution and 3 ml 10% Na₂S₂O₃-solution and stirred vigorously for 15 min at room temperature. The biphasic mixture was then extracted with 30 ml dichloromethane. The organic layer was washed with 5 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated to afford 68 mg (50%; purity=90%) indan-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-2-methoxy-ethyl}-amide as an off-white solid. LC/MS: (M−H)⁻=703.

### Example 24

### Indan-2-carboxylic acid {(S)-1-[(S)-1-((S)-1-benzyl-2-benzylcarbamoyl-2-oxo-ethylcarbamoyl)-3-methyl-butylcarbamoyl]-ethyl}-amide

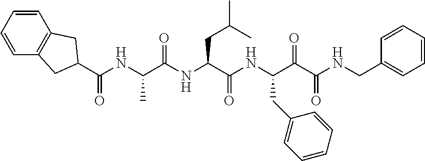 [SMILES: CC(C)C[C@H](NC(=O)[C@H](C)NC(=O)C1Cc2ccccc2C1)C(=O)N[C@@H](Cc1ccccc1)C(=O)C(=O)NCc1ccccc1]

To a solution of Boc-L-alanine (600 mg, 3.17 mmol), L-leucine benzyl ester p-toluenesulfonate salt (1.31 g, 3.33 mmol), and HATU (1.33 g, 3.49 mmol) in DMF (10 ml) was added N,N-diisopropylethylamine (1.23 g, 1.66 ml, 9.51 mmol). The reaction became slightly exothermic so used an ice bath to moderate the temperature. The bright yellow reaction mixture was stirred at room temperature overnight. Quenched with water and extracted with EtOAc (2×). The combined organics were washed with saturated NaHCO₃, water (3×) and brine then dried over MgSO₄ and concentrated. The residue was purified by silica gel chromatography (10% to 30% EtOAc/hexanes) to isolate 1.22 g (98%) (S)-2-((S)-2-tert-butoxycarbonylamino-propionylamino)-4-methyl-pentanoic acid benzyl ester as a viscous colorless oil.

In a 25 ml round-bottomed flask, (S)-2-((S)-2-tert-Butoxycarbonylamino-propionylamino)-4-methyl-pentanoic acid benzyl ester (499 mg, 1.27 mmol) was dissolved in 6.5 ml dichloromethane. Trifluoroacetic acid (2.4 ml, 31.2 mmol) was added slowly. The reaction mixture was stirred at room temperature for 3 h. The solvent was evaporated and then placed under high vacuum for 30 min. The residue (pale yellow oil) was dissolved in 4.5 ml DMF and the solution was stirred at 0° C. for 10 min. N,N-Diisopropylethylamine (1.04 g, 1.4 ml, 8.02 mmol) was added dropwise at 0° C. 2-Indanecarboxylic acid (195 mg, 1.2 mmol) was added followed by HATU (503 mg, 1.32 mmol). After the addition was complete, the ice bath was removed and the reaction mixture was stirred at room temperature overnight. The reaction mixture was diluted with water (an off-white precipitate was formed). The suspension was filtered, rinsing with water and a little petroleum ether. The resulting off-white solid was dried using the rotavap and then placed under high vacuum to afford 455 mg (87%) (S)-2-{(S)-2-[(indane-2-carbonyl)-amino]-propionylamino}-4-methyl-pentanoic acid benzyl ester as an off-white solid.

In a 100 ml round-bottomed flask, (S)-2-{(S)-2-[(Indane-2-carbonyl)-amino]-propionylamino}-4-methyl-pentanoic acid benzyl ester (453 mg, 1.04 mmol) was dissolved in 10 ml methanol. The flask was three times alternating evacuated and flushed with argon. 20% Palladium hydroxide on carbon (wet, 94 mg, 0.13 mmol) was added carefully. The flask was evacuated, flushed with argon, evacuated and flushed with hydrogen. The reaction mixture was stirred under hydrogen atmosphere (balloon) at room temperature for 2 h. The reaction mixture was filtered over Celite, rinsing with ethyl acetate/methanol. The filtrate was concentrated to afford 374 mg (99%; purity=95%) (S)-2-{(S)-2-[(indane-2-carbonyl)-amino]-propionylamino}-4-methyl-pentanoic acid as an off-white foam.

In a 10 ml round-bottomed flask, ((S)-1-benzyl-2-benzylcarbamoyl-2-hydroxy-ethyl)-carbamic acid tert-butyl ester (120 mg, 0.31 mmol) was dissolved in 3.2 ml dichloromethane. Trifluoroacetic acid (0.56 ml, 7.27 mmol) was added slowly. The reaction mixture was stirred at room temperature for 2.5 h. The solvent was evaporated and then put under high vacuum for 30 min. The residue was dissolved in 2.2 ml DMF and the solution was stirred at 0° C. for 5 min. N,N-Diisopropylethylamine (252 mg, 0.34 ml, 1.95 mmol) was added dropwise at 0° C. (S)-2-{(S)-2-[(Indane-2-carbonyl)-amino]-propionylamino}-4-methyl-pentanoic acid (105 mg, 0.29 mmol) was added followed by HATU (120 mg, 0.32 mmol). After the addition was complete, the ice bath was removed and the reaction mixture was stirred at room temperature overnight. The reaction mixture was diluted with water (a light yellow precipitate was formed). The suspension was filtered, rinsing with water and a little petroleum ether. The resulting light yellow solid was dried using the rotavap and then placed under high vacuum to provide 188 mg (96%; purity=90%) indan-2-carboxylic acid {(S)-1-[(S)-1-((S)-1-benzyl-2-benzylcarbamoyl-2-hydroxy-ethylcarbamoyl)-3-methyl-butylcarbamoyl]-ethyl}-amide as a mixture of epimers.

In a 25 ml round-bottomed flask, indan-2-carboxylic acid {(S)-1-[(S)-1-((S)-1-benzyl-2-benzylcarbamoyl-2-hydroxy-ethylcarbamoyl)-3-methyl-butylcarbamoyl]-ethyl}-amide (183 mg, 0.27 mmol) was partially dissolved in 10.0 ml dichloromethane and Dess-Martin periodinane (171 mg, 0.40 mmol) was added (the reaction mixture turned into a yellow suspension). The reaction mixture was stirred at room temperature for 2 h. The reaction mixture was quenched with 4.5 ml saturated NaHCO₃-solution and 4.5 ml 10% Na₂S₂O₃-solution and stirred vigorously for 15 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was absorbed on 2 g silica gel and chromatographed over 12 g silica gel with MeOH/dichloromethane (gradient 0-5% MeOH). All fractions containing product were combined and concentrated. The residue was absorbed on 1 g silica gel and again chromatographed over 4 g silica gel with MeOH/dichloromethane (gradient 0-5% MeOH). All fractions containing product were combined and concentrated. The residue (light yellow solid) was triturated with dichloromethane/diethyl ether to afford 86 mg (50%) indan-2-carboxylic acid {(S)-1-[(S)-1-((S)-1-benzyl-2-benzylcarbamoyl-2-oxo-ethylcarbamoyl)-3-methyl-butylcarbamoyl]-ethyl}-amide as an off-white solid. LC/MS: (M+H)⁺=611.

### Example 25

### 3-Methyl-1H-indene-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide

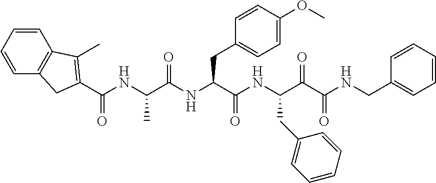 [SMILES: COc1ccc(C[C@H](NC(=O)[C@H](C)NC(=O)C2=C(C)c3ccccc3C2)C(=O)N[C@@H](Cc2ccccc2)C(=O)C(=O)NCc2ccccc2)cc1]

In a 10 ml round-bottomed flask, 3-methyl-1H-indene-2-carboxylic acid (80 mg, 0.46 mmol) and (S)-2-((S)-2-Amino-propionylamino)-3-(4-methoxy-phenyl)-propionic acid methyl ester hydrochloride (150 mg, 0.47 mmol) were dissolved in 2.2 ml DMF. The reaction mixture was cooled to 0° C. N,N-Diisopropylethylamine (178 mg, 0.24 ml, 1.37 mmol) was added dropwise at 0° C. followed by HATU (192 mg, 0.50 mmol). After the addition was complete, the ice bath was removed and the reaction mixture was stirred at room temperature overnight. The reaction mixture was diluted with water (a precipitate was formed). The suspension was filtered, rinsing with water and a little petroleum ether. The resulting light grey solid was dried using the rotavap and then placed under high vacuum to provide 179 mg (89%) (S)-3-(4-methoxy-phenyl)-2-{(S)-2-[(3-methyl-1H-indene-2-carbonyl)-amino]-propionylamino}-propionic acid methyl ester.

In a 25 ml round-bottomed flask, (S)-3-(4-methoxy-phenyl)-2-{(S)-2-[(3-methyl-1H-indene-2-carbonyl)-amino]-propionylamino}-propionic acid methyl ester (176 mg, 0.40 mmol) was partially dissolved in 4.0 ml 1,2-dichloroethane. Trimethyltin hydroxide (292 mg, 1.61 mmol) was added and the reaction mixture was stirred at 80° C. for 4 h. The reaction mixture was cooled to room temperature and then concentrated. The residue was taken up in 30 ml ethyl acetate and 3 ml 1M HCl. The aqueous layer was extracted with 30 ml ethyl acetate. The organic layers were washed twice with 3 ml 1M HCl, once with 3 ml water and once with 3 ml brine. The organic layers were combined, dried over sodium sulfate, filtered and concentrated. The residue was triturated with dichloromethane/hexanes to afford 143 mg (84%) (S)-3-(4-methoxy-phenyl)-2-{(S)-2-[(3-methyl-1H-indene-2-carbonyl)-amino]-propionylamino}-propionic acid as a light brown solid.

In a 10 ml round-bottomed flask, ((S)-1-Benzyl-2-benzylcarbamoyl-2-hydroxy-ethyl)-carbamic acid tert-butyl ester (130 mg, 0.34 mmol) was dissolved in 3.0 ml dichloromethane. Trifluoroacetic acid (0.68 ml, 8.83 mmol) was added slowly. The reaction mixture was stirred at room temperature for 3 h. The solvent was evaporated and then put under high vacuum for 15 min. The residue was dissolved in 2.0 ml DMF and the solution was stirred at 0° C. for 10 min. N,N-Diisopropylethylamine (266 mg, 0.36 ml, 2.06 mmol) was added dropwise at 0° C. (S)-3-(4-Methoxy-phenyl)-2-{(S)-2-[(3-methyl-1H-indene-2-carbonyl)-amino]-propionylamino}-propionic acid (130 mg, 0.31 mmol) was added followed by HATU (129 mg, 0.34 mmol). After the addition was complete, the ice bath was removed and the reaction mixture was stirred at room temperature overnight (a precipitate was formed). The reaction mixture was diluted with water. The suspension was filtered, rinsing with water and a little petroleum ether. The resulting off-white solid was dried using the rotavap and then placed under high vacuum to afford 212 mg (95%; purity=95%) 3-methyl-1H-indene-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide as a mixture of epimers.

In a 50 ml round-bottomed flask, 3-methyl-1H-indene-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide (208 mg, 0.29 mmol) was partially dissolved in 13 ml dichloromethane and Dess-Martin periodinane (183 mg, 0.43 mmol) was added. The reaction mixture was stirred at room temperature for 2.5 h. The reaction mixture was quenched with 4.5 ml saturated NaHCO₃-solution and 4.5 ml 10% Na₂S₂O₃-solution and stirred vigorously for 15 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue was triturated with dichloromethane/hexanes to afford 82 mg (38%; purity=90%) 3-methyl-1H-indene-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide as an off-white solid. LC/MS: (M−H)⁻=685.

### Example 26

### Indan-2-carboxylic acid {(S)-[(S)-1-((S)-1-benzyl-2-benzylcarbamoyl-2-oxo-ethylcarbamoyl)-2-(4-methoxy-phenyl)-ethylcarbamoyl]-cyclopropyl-methyl}-amide

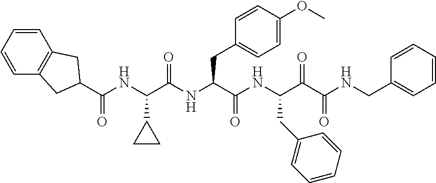 [SMILES: COc1ccc(C[C@H](NC(=O)[C@@H](NC(=O)C2Cc3ccccc3C2)C2CC2)C(=O)N[C@@H](Cc2ccccc2)C(=O)C(=O)NCc2ccccc2)cc1]

In a 25 ml round-bottomed flask, (S)-2-tert-butoxycarbonylamino-3-(4-methoxy-phenyl)-propionic acid benzyl ester (360 mg, 0.93 mmol) was dissolved in 3.2 ml dichloromethane. Trifluoroacetic acid (1.8 ml, 23.4 mmol) was added slowly. The reaction mixture was stirred at room temperature for 2.5 h. The solvent was evaporated and then placed under high vacuum for 15 min. The residue (colorless oil) was dissolved in 2.1 ml DMF and the solution was stirred at 0° C. for 10 min. N,N-Diisopropylethylamine (666 mg, 0.9 ml, 5.15 mmol) was added dropwise at 0° C. Boc-L-cyclopropylglycine (175 mg, 0.81 mmol) was added followed by HATU (340 mg, 0.89 mmol). After the addition was complete, the ice bath was removed and the reaction mixture was stirred at room temperature overnight. The reaction mixture was extracted with 40 ml diethyl ether and 3 ml water. The aqueous layer was extracted with 40 ml diethyl ether. The organic layers were washed three times with 3 ml water and once with 3 ml brine. The organic layers were combined, dried over sodium sulfate, filtered and concentrated to afford (S)-2-((S)-2-tert-butoxycarbonylamino-2-cyclopropyl-acetylamino)-3-(4-methoxy-phenyl)-propionic acid benzyl ester as a light yellow oil which was used without further purification.

In a 25 ml round-bottomed flask, (S)-2-((S)-2-tert-butoxycarbonylamino-2-cyclopropyl-acetylamino)-3-(4-methoxy-phenyl)-propionic acid benzyl ester (526 mg, 0.76 mmol) was dissolved in 4.2 ml dichloromethane. Trifluoroacetic acid (1.7 ml, 22.1 mmol) was added slowly. The reaction mixture was stirred at room temperature for 2.5 h. The solvent was evaporated and then placed under high vacuum for 15 min. The residue (off-white solid) was dissolved in 2.8 ml and the solution was stirred at 0° C. for 10 min. N,N-Diisopropylethylamine (577 mg, 0.78 ml, 4.47 mmol) was added dropwise at 0° C. 2-Indanecarboxylic acid (120 mg, 0.74 mmol) was added followed by HATU (309 mg, 0.81 mmol). After the addition was complete, the ice bath was removed. A thick precipitate formed, so 2.0 ml DMF was added and the yellow suspension was stirred at room temperature overnight. The reaction mixture was diluted with water. The suspension was filtered, rinsing with water and a little petroleum ether. The resulting off-white solid was dried using the rotavap and then placed under high vacuum to afford 372 mg (95%) (S)-2-{(S)-2-cyclopropyl-2-[(indane-2-carbonyl)-amino]-acetylamino}-3-(4-methoxy-phenyl)-propionic acid benzyl ester.

In a 50 ml round-bottomed flask, (S)-2-{(S)-2-cyclopropyl-2-[(indane-2-carbonyl)-amino]-acetylamino}-3-(4-methoxy-phenyl)-propionic acid benzyl ester (370 mg, 0.70 mmol) was partially dissolved in 10 ml methanol, 10 ml THF and 2.0 ml DMF. The flask was three times alternating evacuated and flushed with argon. 20% Palladium hydroxide on carbon (wet, 70 mg, 0.10 mmol) was added carefully. The flask was evacuated, flushed with argon, evacuated and flushed with hydrogen. The reaction mixture was stirred under hydrogen atmosphere (balloon) at 40° C. (=oil bath temperature) for 1 h. The reaction mixture was filtered over Celite, rinsing with ethyl acetate/methanol. The filtrate was concentrated and then put under high vacuum. The residue (pale light solid) was triturated with dichloromethane/hexanes to afford 286 mg (93%) (S)-2-{(S)-2-cyclopropyl-2-[(indane-2-carbonyl)-amino]-acetylamino}-3-(4-methoxy-phenyl)-propionic acid as a white solid.

In a 10 ml round-bottomed flask, ((S)-1-benzyl-2-benzylcarbamoyl-2-hydroxy-ethyl)-carbamic acid tert-butyl ester (130 mg, 0.34 mmol) was dissolved in 3.0 ml dichloromethane. Trifluoroacetic acid (0.68 ml, 8.83 mmol) was added slowly. The reaction mixture was stirred at room temperature for 2.5 h. The solvent was evaporated and then put under high vacuum for 30 min. The residue was dissolved in 2.0 ml DMF and the solution was stirred at 0° C. for 10 min. N,N-Diisopropylethylamine (266 mg, 0.36 ml, 2.06 mmol) was added dropwise at 0° C. (S)-2-{(S)-2-Cyclopropyl-2-[(indane-2-carbonyl)-amino]-acetylamino}-3-(4-methoxy-phenyl)-propionic acid (134 mg, 0.31 mmol) was added followed by HATU (128 mg, 0.34 mmol). After the addition was complete, the ice bath was removed and the reaction mixture was stirred at room temperature overnight (a precipitate was formed). The reaction mixture was diluted with water. The suspension was filtered, rinsing with water and a little petroleum ether. The resulting off-white solid was dried using the rotavap and then placed under high vacuum to afford 198 mg (92%) indan-2-carboxylic acid {(S)-[(S)-1-((S)-1-benzyl-2-benzylcarbamoyl-2-hydroxy-ethylcarbamoyl)-2-(4-methoxy-phenyl)-ethylcarbamoyl]-cyclopropyl-methyl}-amide as a mixture of epimers.

In a 50 ml round-bottomed flask, indan-2-carboxylic acid {(S)-[(S)-1-((S)-1-benzyl-2-benzylcarbamoyl-2-hydroxy-ethylcarbamoyl)-2-(4-methoxy-phenyl)-ethylcarbamoyl]-cyclopropyl-methyl}-amide (194 mg, 0.28 mmol) was partially dissolved in 12 ml dichloromethane and Dess-Martin periodinane (176 mg, 0.41 mmol) was added. The reaction mixture was stirred at room temperature for 2.5 h. The reaction mixture was quenched with 4.5 ml saturated NaHCO₃-solution and 4.5 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue was triturated with dichloromethane/hexanes to afford 106 mg (49%; purity=90%) indan-2-carboxylic acid {(S)-[(S)-1-((S)-1-benzyl-2-benzylcarbamoyl-2-oxo-ethylcarbamoyl)-2-(4-methoxy-phenyl)-ethylcarbamoyl]-cyclopropyl-methyl}-amide as an off-white solid. LC/MS: (M−H)⁻=699.

### Example 27

### Indan-2-carboxylic acid {1-[(S)-1-((S)-1-benzyl-2-benzylcarbamoyl-2-oxo-ethylcarbamoyl)-2-(4-methoxy-phenyl)-ethylcarbamoyl]-cyclopropyl}-amide

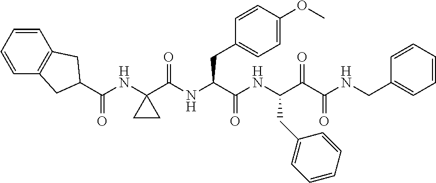 [SMILES: COc1ccc(C[C@H](NC(=O)C2(NC(=O)C3Cc4ccccc4C3)CC2)C(=O)N[C@@H](Cc2ccccc2)C(=O)C(=O)NCc2ccccc2)cc1]

In a 25 ml round-bottomed flask, (S)-2-tert-butoxycarbonylamino-3-(4-methoxy-phenyl)-propionic acid benzyl ester (548 mg, 1.42 mmol) was dissolved in 5.2 ml dichloromethane. Trifluoroacetic acid (2.8 ml, 36.3 mmol) was added slowly. The reaction mixture was stirred at room temperature for 3 h. The solvent was evaporated and then placed under high vacuum for 30 min. The residue (colorless oil) was dissolved in 3.4 ml DMF and the solution was stirred at 0° C. for 10 min. N,N-Diisopropylethylamine (1.18 g, 1.6 ml, 9.16 mmol) was added dropwise at 0° C. 1-(Boc-amino)cyclopropanecarboxylic acid (260 mg, 1.29 mmol) was added followed by HATU (540 mg, 1.42 mmol). After the addition was complete, the ice bath was removed and the reaction mixture was stirred at room temperature over the weekend. The reaction mixture was extracted with 70 ml diethyl ether and 5 ml water. The aqueous layer was extracted with 70 ml diethyl ether. The organic layers were washed three times with 5 ml water and once with 5 ml brine. The organic layers were combined, dried over sodium sulfate, filtered and concentrated to provide (S)-2-[(1-tert-butoxycarbonylamino-cyclopropanecarbonyl)-amino]-3-(4-methoxy-phenyl)-propionic acid benzyl ester as a brown oil which was used without further purification.

In a 25 ml round-bottomed flask, (S)-2-[(1-tert-butoxycarbonylamino-cyclopropanecarbonyl)-amino]-3-(4-methoxy-phenyl)-propionic acid benzyl ester (800 mg, 1.2 mmol; purity=70%) was dissolved in 6.2 ml dichloromethane. Trifluoroacetic acid (2.5 ml, 32.4 mmol) was added slowly. The reaction mixture was stirred at room temperature for 2.5 h. The solvent was evaporated and then put under high vacuum for 30 min. The residue (brown oil) was dissolved in 4.2 ml DMF and the solution was stirred at 0° C. for 10 min. N,N-Diisopropylethylamine (888 mg, 1.2 ml, 6.87 mmol) was added dropwise at 0° C. 2-Indanecarboxylic acid (178 mg, 1.1 mmol) was added followed by HATU (459 mg, 1.21 mmol). After the addition was complete, the ice bath was removed and the reaction mixture was stirred at room temperature overnight. The reaction mixture was extracted with 70 ml diethyl ether and 5 ml water. The aqueous layer was extracted with 70 ml diethyl ether. The organic layers were washed twice with 5 ml water and once with 5 ml brine. The organic layers were combined, dried over sodium sulfate, filtered and concentrated. The residue (brown oil) was chromatographed over 25 g silica gel with EtOAc/hexanes (gradient 0-50% EtOAc). All fractions containing product were combined and concentrated to afford 518 mg (92%) (S)-2-({1-[(indane-2-carbonyl)-amino]-cyclopropanecarbonyl}-amino)-3-(4-methoxy-phenyl)-propionic acid benzyl ester as a light yellow foam.

In a 50 ml round-bottomed flask, (S)-2-({1-[(indane-2-carbonyl)-amino]-cyclopropanecarbonyl}-amino)-3-(4-methoxy-phenyl)-propionic acid benzyl ester (514 mg, 1.00 mmol) was dissolved in 10 ml methanol. The flask was three times alternating evacuated and flushed with argon. 20% Palladium hydroxide on carbon (wet, 91 mg, 0.13 mmol) was added carefully. The flask was evacuated, flushed with argon, evacuated and flushed with hydrogen. The reaction mixture was stirred under hydrogen atmosphere (balloon) at room temperature for 2.5 h. The reaction mixture was filtered over Celite, rinsing with ethyl acetate/methanol. The filtrate was concentrated to afford 416 mg (98%) (S)-2-({1-[(indane-2-carbonyl)-amino]-cyclopropanecarbonyl}-amino)-3-(4-methoxy-phenyl)-propionic acid as an off-white foam.

In a 10 ml round-bottomed flask, ((S)-1-benzyl-2-benzylcarbamoyl-2-hydroxy-ethyl)-carbamic acid tert-butyl ester (130 mg, 0.34 mmol) was dissolved in 3.0 ml dichloromethane. Trifluoroacetic acid (0.68 ml, 8.83 mmol) was added slowly. The reaction mixture was stirred at room temperature for 2.5 h. The solvent was evaporated and then placed under high vacuum for 30 min. The residue dissolved in 2.0 ml DMF and the solution was stirred at 0° C. for 10 min. N,N-Diisopropylethylamine (266 mg, 0.36 ml, 2.06 mmol) was added dropwise at 0° C. (S)-2-({1-[(Indane-2-carbonyl)-amino]-cyclopropanecarbonyl}-amino)-3-(4-methoxy-phenyl)-propionic acid (130 mg, 0.31 mmol) was added followed by HATU (129 mg, 0.34 mmol). After the addition was complete, the ice bath was removed and the reaction mixture was stirred at room temperature overnight. The reaction mixture was extracted with 40 ml diethyl ether and 5 ml water. The aqueous layer was extracted with 40 ml diethyl ether. The organic layers were washed twice with 5 ml water and once with 5 ml brine. The organic layers were combined, dried over sodium sulfate, filtered and concentrated. The residue was chromatographed over 12 g silica gel with MeOH/dichloromethane (gradient 0-5% MeOH). All fractions containing product were combined and concentrated to provide 204 mg (96%) indan-2-carboxylic acid {1-[(S)-1-((S)-1-benzyl-2-benzylcarbamoyl-2-hydroxy-ethylcarbamoyl)-2-(4-methoxy-phenyl)-ethylcarbamoyl]-cyclopropyl}-amide as an off-white solid and as a mixture of epimers.

In a 25 ml round-bottomed flask, indan-2-carboxylic acid {1-[(S)-1-((S)-1-benzyl-2-benzylcarbamoyl-2-hydroxy-ethylcarbamoyl)-2-(4-methoxy-phenyl)-ethylcarbamoyl]-cyclopropyl}-amide (200 mg, 0.29 mmol) was dissolved in 13 ml dichloromethane and Dess-Martin periodinane (185 mg, 0.44 mmol) was added. The reaction mixture was stirred at room temperature for 2.5 h. The reaction mixture was quenched with 4.5 ml saturated NaHCO₃-solution and 4.5 ml 10% Na₂S₂O₃-solution and stirred vigorously for 15 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue was triturated with dichloromethane/hexanes to afford 80 mg (36%; purity=90%) indan-2-carboxylic acid {1-[(S)-1-((S)-1-benzyl-2-benzylcarbamoyl-2-oxo-ethylcarbamoyl)-2-(4-methoxy-phenyl)-ethylcarbamoyl]-cyclopropyl}-amide as a white solid. LC/MS: (M−H)⁻=685.

### Example 28

### {1-[(S)-1-((S)-1-Benzyl-2-benzylcarbamoyl-2-oxo-ethylcarbamoyl)-2-(4-methoxy-phenyl)-ethylcarbamoyl]-cyclopropyl}-carbamic acid benzyl ester

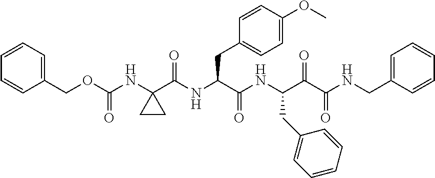 [SMILES: COc1ccc(C[C@H](NC(=O)C2(NC(=O)OCc3ccccc3)CC2)C(=O)N[C@@H](Cc2ccccc2)C(=O)C(=O)NCc2ccccc2)cc1]

To a solution of (S)-2-tert-butoxycarbonylamino-3-(4-methoxy-phenyl)-propionic acid methyl ester (524 mg, 1.69 mmol) in CH₂Cl₂ (8 ml) was added TFA (3 ml). The reaction mixture was stirred room temperature for 3 h then concentrated, chased with hexanes (2×) and dried under high vacuum. The residue was dissolved in DMF (5 ml) and cooled to 0° C. Then added 1-(benzyloxycarbonylamino)cyclopropanecarboxylic acid (340 mg, 1.45 mmol) and HATU (605 mg, 1.59 mmol) followed by N,N-diisopropylethylamine (1.26 ml, 7.23 mmol). The reaction mixture was warmed to room temperature and stirred overnight then quenched with water and extracted with EtOAc (2×). The combined organics were washed with water (3×) and brine then dried over MgSO₄ and concentrated. The residue was purified by silica gel chromatography (20% to 50% EtOAc/hexanes) to afford 550 mg (89%) of (S)-2-[(1-benzyloxycarbonylamino-cyclopropanecarbonyl)-amino]-3-(4-methoxy-phenyl)-propionic acid methyl ester as a white foam.

To a solution of (S)-2-[(1-benzyloxycarbonylamino-cyclopropanecarbonyl)-amino]-3-(4-methoxy-phenyl)-propionic acid methyl ester (550 mg, 1.29 mmol) in 1,2-dichloroethane (12 ml) was added trimethyltin hydroxide (933 mg, 5.16 mmol). The cloudy reaction mixture was stirred at 80° C. for 6 h then cooled to room temperature and concentrated under reduced pressure. The residue was partitioned between EtOAc and 1.0M HCl. The aqueous layer was extracted with EtOAc. The combined organics were washed with 1.0M HCl (5×) then dried over MgSO₄ and concentrated to provide (S)-2-[(1-benzyloxycarbonylamino-cyclopropanecarbonyl)-amino]-3-(4-methoxy-phenyl)-propionic acid as a colorless semisolid which was used without further purification.

To a solution of ((S)-1-benzyl-2-benzylcarbamoyl-2-hydroxy-ethyl)-carbamic acid tert-butyl ester (120 mg, 0.31 mmol) in CH₂Cl₂ (4 ml) was added trifluoroacetic acid (1.0 ml). The reaction mixture was stirred at room temperature for 2 h then concentrated and chased with hexanes. To the residue was added a solution of (S)-2-[(1-benzyloxycarbonylamino-cyclopropanecarbonyl)-amino]-3-(4-methoxy-phenyl)-propionic acid (150 mg, 0.26 mmol) in DMF (3 ml) and HATU (106 mg, 0.28 mmol). The mixture was cooled to 0° C. and N,N-diisopropylethylamine (0.36 ml, 2.04 mmol) was added. The reaction was warmed to room temperature and stirred overnight then quenched with water and extracted with EtOAc (2×). The combined organics were washed with water (3×) and brine then dried over MgSO₄ and concentrated. The residue was absorbed on silica gel and purified by chromatography (30% to 100% EtOAc/hexanes) to give 106 mg (61%) of {1-[(S)-1-((S)-1-benzyl-2-benzylcarbamoyl-2-hydroxy-ethylcarbamoyl)-2-(4-methoxy-phenyl)-ethylcarbamoyl]-cyclopropyl}-carbamic acid benzyl ester as a white foamy solid and a mixture of epimers. LC/MS (M-OBn)⁻=569.

To a solution of {1-[(S)-1-((S)-1-benzyl-2-benzylcarbamoyl-2-hydroxy-ethylcarbamoyl)-2-(4-methoxy-phenyl)-ethylcarbamoyl]-cyclopropyl}-carbamic acid benzyl ester (103 mg, 0.15 mmol) in CH₂Cl₂ (6 ml) was added Dess-Martin periodinane (97 mg, 0.23 mmol). The reaction mixture was stirred at room temperature for 2 h during which time a fine precipitate was formed. Added 10% aqueous Na₂S₂O₃ (5 ml) and sat'd NaHCO₃ (5 mL). The biphasic mixture was stirred vigorously for 20 min then diluted with water and extracted with CH₂Cl₂ (2×). The combined organics were washed with sat'd NaHCO₃ and concentrated. The residue was triturated with Et₂O to afford 77 mg (75%) of {1-[(S)-1-((S)-1-benzyl-2-benzylcarbamoyl-2-oxo-ethylcarbamoyl)-2-(4-methoxy-phenyl)-ethylcarbamoyl]-cyclopropyl}-carbamic acid benzyl ester as an off-white solid. LC/MS: (M+H)⁺=677.

### Example 29

### Pyrazine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide

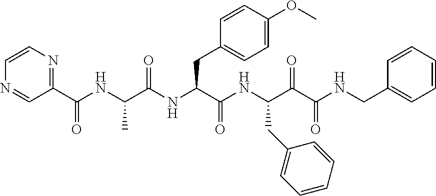 [SMILES: COc1ccc(C[C@H](NC(=O)[C@H](C)NC(=O)c2cnccn2)C(=O)N[C@@H](Cc2ccccc2)C(=O)C(=O)NCc2ccccc2)cc1]

In a 25 ml round-bottomed flask, (S)-2-((S)-2-tert-butoxycarbonylamino-propionylamino)-3-(4-methoxy-phenyl)-propionic acid benzyl ester (600 mg, 1.25 mmol) was dissolved in 7.0 ml dichloromethane. Trifluoroacetic acid (2.7 ml, 35.1 mmol) was added slowly. The reaction mixture was stirred at room temperature for 2.5 h. The solvent was evaporated and then placed under high vacuum for 15 min. The residue (light brown solid) was dissolved in 4.6 ml DMF and the solution was stirred at 0° C. for 10 min. N,N-Diisopropylethylamine (962 mg, 1.3 ml, 7.44 mmol) was added dropwise at 0° C. Pyrazine-2-carboxylic acid (145 mg, 1.17 mmol) was added followed by HATU (489 mg, 1.29 mmol). After the addition was complete, the ice bath was removed and the reaction mixture was stirred at room temperature overnight. The reaction mixture was extracted with 70 ml diethyl ether and 5 ml water. The aqueous layer was extracted with 70 ml diethyl ether. The organic layers were washed twice with 5 ml water and once with 5 ml brine. The organic layers were combined, dried over sodium sulfate, filtered and concentrated. The residue was chromatographed over 25 g silica gel with EtOAc/hexanes (gradient 0-80% EtOAc). All fractions containing product were combined and concentrated to provide 615 mg (97%; purity=85%) (S)-3-(4-methoxy-phenyl)-2-{(S)-2-[(pyrazine-2-carbonyl)-amino]-propionylamino}-propionic acid benzyl ester as an off-white solid.

In a 50 ml round-bottomed flask, (S)-3-(4-methoxy-phenyl)-2-{(S)-2-[(pyrazine-2-carbonyl)-amino]-propionylamino}-propionic acid benzyl ester (592 mg, 1.09 mmol; purity=85%) was dissolved in 6.0 ml methanol and 6.0 ml THF. The flask was three times alternating evacuated and flushed with argon. 20% Palladium hydroxide on carbon (wet, 100 mg, 0.14 mmol) was added carefully. The flask was evacuated, flushed with argon, evacuated and flushed with hydrogen. The reaction mixture was stirred under hydrogen atmosphere (balloon) at room temperature for 1.5 h. The reaction mixture was filtered over Celite, rinsing with ethyl acetate/methanol. The filtrate was concentrated to afford (S)-3-(4-methoxy-phenyl)-2-{(S)-2-[(pyrazine-2-carbonyl)-amino]-propionylamino}-propionic acid as a dark brown oil which was used without further purification.

In a 10 ml round-bottomed flask, ((S)-1-benzyl-2-benzylcarbamoyl-2-hydroxy-ethyl)-carbamic acid tert-butyl ester (130 mg, 0.34 mmol) was dissolved in 3.0 ml dichloromethane. Trifluoroacetic acid (0.68 ml, 8.83 mmol) was added slowly. The reaction mixture was stirred at room temperature for 3.5 h. The solvent was evaporated and then placed under high vacuum for 30 min. The residue and (S)-3-(4-methoxy-phenyl)-2-{(S)-2-[(pyrazine-2-carbonyl)-amino]-propionylamino}-propionic acid (150 mg, 0.32 mmol; purity=80%) were dissolved in 3.0 ml DMF and the dark brown solution was cooled to 0° C. N,N-Diisopropylethylamine (266 mg, 0.36 ml, 2.06 mmol) was added dropwise at 0° C. followed by HATU (135 mg, 0.35 mmol). After the addition was complete, the ice bath was removed and the reaction mixture was stirred at room temperature overnight. The reaction mixture was extracted with 60 ml diethyl ether/ethyl acetate (2:1) and 5 ml water. The aqueous layer was extracted with 60 ml Diethyl ether/Ethyl acetate (2:1). The organic layers were washed three times with 5 ml water and once with 5 ml brine. The organic layers were combined, dried over sodium sulfate, filtered and concentrated. The aqueous layers were combined and extracted three times with 30 ml ethyl acetate. The organic layers were combined, dried over sodium sulfate, filtered, combined with the residue from the first extraction and concentrated. The residue was triturated with methanol. The solid was set aside. The filtrate was concentrated and chromatographed over 12 g silica gel with MeOH/dichloromethane (gradient 0-5% MeOH). All fractions containing product were combined with the solid from the trituration and concentrated to provide 151 mg (73%) pyrazine-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide as a light brown solid and as a mixture of epimers.

In a 25 ml round-bottomed flask, pyrazine-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide (148 mg, 0.23 mmol) was partially dissolved in 10.0 ml dichloromethane and Dess-Martin periodinane (147 mg, 0.35 mmol) was added. The reaction mixture was stirred at room temperature for 2 h. The reaction mixture was quenched with 4 ml saturated NaHCO₃-solution and 4 ml 10% Na₂S₂O₃-solution and stirred vigorously for 15 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue was triturated with dichloromethane/hexanes to afford 81 mg (49%; purity=90%) pyrazine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide as an off-white solid. LC/MS: (M−H)⁻=635.

### Example 30

### 2-Methyl-2H-pyrazole-3-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide

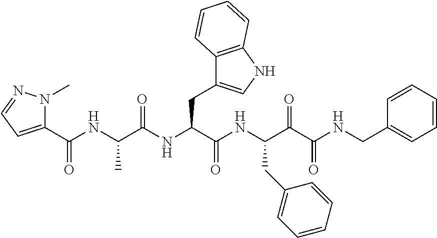 [SMILES: C[C@H](NC(=O)c1ccnn1C)C(=O)N[C@@H](Cc1c[nH]c2ccccc12)C(=O)N[C@@H](Cc1ccccc1)C(=O)C(=O)NCc1ccccc1]

In a 10 ml round-bottomed flask, ((S)-1-benzyl-2-benzylcarbamoyl-2-hydroxy-ethyl)-carbamic acid tert-butyl ester (120 mg, 0.31 mmol) was dissolved in 3.0 ml dichloromethane. Trifluoroacetic acid (0.63 ml, 8.18 mmol) was added slowly. The reaction mixture was stirred at room temperature for 3 h. The solvent was evaporated and then placed under high vacuum for 30 min. The residue and (S)-3-(1H-indol-3-yl)-2-{(S)-2-[(2-methyl-2H-pyrazole-3-carbonyl)-amino]-propionylamino}-propionic acid (190 mg, 0.27 mmol; purity=55%) were dissolved in 2.0 ml DMF and the brown solution was cooled to 0° C. N,N-Diisopropylethylamine (222 mg, 0.30 ml, 1.72 mmol) was added dropwise at 0° C. followed by HATU (114 mg, 0.30 mmol). After the addition was complete, the ice bath was removed and the reaction mixture was stirred at room temperature for 2 d. The reaction mixture was extracted with diethyl ether/EtOAc (1:1) and water. The aqueous layer was back extracted with diethyl ether/EtOAc (1:1). The organic layers were washed twice with water and once with brine. The organic layers were combined, dried over sodium sulfate, filtered and concentrated. The residue was chromatographed over 12 g silica gel with MeOH/dichloromethane (gradient 0-5% MeOH). All fractions containing product were combined and concentrated to afford 118 mg (67%) 2-methyl-2H-pyrazole-3-carboxylic acid {(S)-1-[(S)-1-((S)-1-benzyl-2-benzylcarbamoyl-2-hydroxy-ethylcarbamoyl)-2-(1H-indol-3-yl)-ethylcarbamoyl]-ethyl}-amide as an off-white solid and as a mixture of epimers.

In a 25 ml round-bottomed flask, 2-methyl-2H-pyrazole-3-carboxylic acid {(S)-1-[(S)-1-((S)-1-benzyl-2-benzylcarbamoyl-2-hydroxy-ethylcarbamoyl)-2-(1H-indol-3-yl)-ethylcarbamoyl]-ethyl}-amide (116 mg, 0.18 mmol) was partially dissolved in 8.0 ml dichloromethane and Dess-Martin periodinane (114 mg, 0.27 mmol) was added. The reaction mixture was stirred at room temperature for 2.5 h. The reaction mixture was quenched with 3 ml saturated NaHCO₃-solution and 3 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was absorbed on 2 g silica gel and chromatographed over 12 g silica gel with MeOH/dichloromethane (gradient 0-5% MeOH). All fractions containing product were combined and concentrated. The residue was triturated with dichloromethane/hexanes to afford 34 mg (26%; purity=90%) 2-methyl-2H-pyrazole-3-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide as a light brown solid. LC/MS: (M−H)⁻=648.

### Example 31

### {(S)-1-[(S)-1-((S)-1-Benzyl-2-methylcarbamoyl-2-oxo-ethylcarbamoyl)-2-(1H-indol-3-yl)-ethylcarbamoyl]-ethyl}-carbamic acid benzyl ester

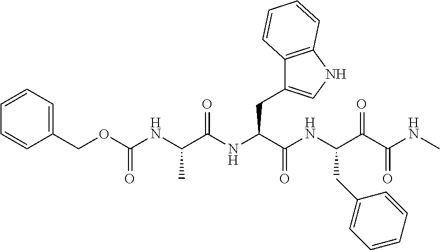 [SMILES: CNC(=O)C(=O)[C@H](Cc1ccccc1)NC(=O)[C@H](Cc1c[nH]c2ccccc12)NC(=O)[C@H](C)NC(=O)OCc1ccccc1]

A 10 ml round-bottomed flask was charged with (S)-3-tert-butoxycarbonylamino-2-hydroxy-4-phenyl-butyric acid (200 mg, 0.64 mmol), methylamine hydrochloride (61 mg, 0.90 mmol) and 2.5 ml dichloromethane. N,N-Diisopropylethylamine (237 mg, 0.32 ml, 1.83 mmol) was added followed by HATU (269 mg, 0.71 mmol). The yellow solution was stirred at room temperature overnight. The reaction mixture was extracted with 5 ml water and 30 ml dichloromethane. The aqueous layers were extracted with 30 ml dichloromethane. The organic layers were combined, dried over sodium sulfate, filtered and concentrated. The residue was chromatographed over 12 g silica gel with EtOAc/hexanes (gradient 0-80% EtOAc). All fractions containing product were combined and concentrated to afford 178 mg (72%; purity=80%) ((S)-1-benzyl-2-hydroxy-2-methylcarbamoyl-ethyl)-carbamic acid tert-butyl ester as an off-white solid and as a mixture of epimers.

In a 10 ml round-bottomed flask, ((S)-1-benzyl-2-hydroxy-2-methylcarbamoyl-ethyl)-carbamic acid tert-butyl ester (170 mg, 0.44 mmol; purity=80%) was dissolved in 3.0 ml dichloromethane. Trifluoroacetic acid (0.76 ml, 9.86 mmol) was added slowly. The reaction mixture was stirred at room temperature for 2.5 h. The solvent was evaporated and then placed under high vacuum for 30 min. The residue (light yellow oil) was dissolved in 2.0 ml DMF and cooled to 0° C. N,N-Diisopropylethylamine (259 mg, 0.35 ml, 2.00 mmol) was added dropwise at 0° C. followed by (S)-2-((S)-2-benzyloxycarbonylamino-propionylamino)-3-(1H-indol-3-yl)-propionic acid (170 mg, 0.33 mmol; purity=80%) and HATU (139 mg, 0.37 mmol). After the addition was complete, the ice bath was removed and the reaction mixture was stirred at room temperature overnight. The reaction mixture was diluted with water (a precipitate was formed). The suspension was filtered, rinsing with water and a little petroleum ether. The resulting off-white solid was dried using the rotavap and then placed under high vacuum to provide 164 mg (82%) {(S)-1-[(S)-1-((S)-1-benzyl-2-hydroxy-2-methylcarbamoyl-ethylcarbamoyl)-2-(1H-indol-3-yl)-ethylcarbamoyl]-ethyl}-carbamic acid benzyl ester as a mixture of epimers.

In a 25 ml round-bottomed flask, {(S)-1-[(S)-1-((S)-1-benzyl-2-hydroxy-2-methylcarbamoyl-ethylcarbamoyl)-2-(1H-indol-3-yl)-ethylcarbamoyl]-ethyl}-carbamic acid benzyl ester (161 mg, 0.27 mmol) was partially dissolved in 11 ml dichloromethane and Dess-Martin periodinane (171 mg, 0.40 mmol) was added. The reaction mixture was stirred at room temperature for 2 h (the reaction mixture turned into a dark brown solution). The reaction mixture was quenched with 4.5 ml saturated NaHCO₃-solution and 4.5 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was triturated with ethyl acetate and a minimal amount of diethyl ether to afford 46 mg (27%) {(S)-1-[(S)-1-((S)-1-benzyl-2-methylcarbamoyl-2-oxo-ethylcarbamoyl)-2-(1H-indol-3-yl)-ethylcarbamoyl]-ethyl}-carbamic acid benzyl ester as a brown solid. LC/MS: (M+H)⁺=598.

### Example 32

### {(S)-1-[(S)-1-((S)-1-Benzyl-2-cyclopropylcarbamoyl-2-oxo-ethylcarbamoyl)-2-(1H-indol-3-yl)-ethylcarbamoyl]-ethyl}-carbamic acid benzyl ester

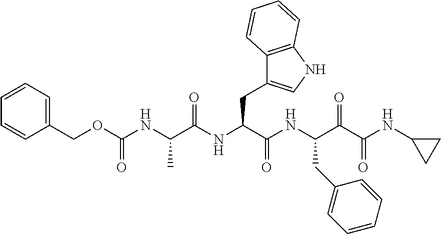 [SMILES: C[C@H](NC(=O)OCc1ccccc1)C(=O)N[C@@H](Cc1c[nH]c2ccccc12)C(=O)N[C@@H](Cc1ccccc1)C(=O)C(=O)NC1CC1]

A 10 ml round-bottomed flask was charged with (S)-3-tert-butoxycarbonylamino-2-hydroxy-4-phenyl-butyric acid (200 mg, 0.64 mmol), cyclopropylamine (56 mg, 0.98 mmol) and 2.5 ml dichloromethane. N,N-Diisopropylethylamine (170 mg, 0.23 ml, 1.32 mmol) was added followed by HATU (269 mg, 0.71 mmol). The yellow solution was stirred at room temperature overnight. The reaction mixture was extracted with 5 ml water and 30 ml dichloromethane. The aqueous layers were extracted with 30 ml dichloromethane. The organic layers were combined, dried over sodium sulfate, filtered and concentrated. The residue was chromatographed over 12 g silica gel with EtOAc/hexanes (gradient 0-70% EtOAc). All fractions containing product were combined and concentrated to afford 61 mg (28%) ((S)-1-benzyl-2-cyclopropylcarbamoyl-2-hydroxy-ethyl)-carbamic acid tert-butyl ester as an off-white waxy solid and as a mixture of epimers.

In a 10 ml round-bottomed flask, ((S)-1-benzyl-2-cyclopropylcarbamoyl-2-hydroxy-ethyl)-carbamic acid tert-butyl ester (60 mg, 0.18 mmol) was dissolved in 1.5 ml dichloromethane. Trifluoroacetic acid (0.31 ml, 4.02 mmol) was added slowly. The reaction mixture was stirred at room temperature for 2.5 h. The solvent was evaporated and then placed under high vacuum for 15 min. The residue (light yellow oil) was dissolved in 1.0 ml DMF and cooled to 0° C. N,N-Diisopropylethylamine (104 mg, 0.14 ml, 0.80 mmol) was added dropwise at 0° C. followed by (S)-2-((S)-2-benzyloxycarbonylamino-propionylamino)-3-(1H-indol-3-yl)-propionic acid (70 mg, 0.14 mmol; purity=80%) and HATU (58 mg, 0.15 mmol). After the addition was complete, the ice bath was removed and the reaction mixture was stirred at room temperature overnight. The reaction mixture was diluted with water (a precipitate was formed). The suspension was filtered, rinsing with water and a little petroleum ether. The resulting light yellow solid was dried using the rotavap and then placed under high vacuum to afford 67 mg (78%) {(S)-1-[(S)-1-((S)-1-benzyl-2-cyclopropylcarbamoyl-2-hydroxy-ethylcarbamoyl)-2-(1H-indol-3-yl)-ethylcarbamoyl]-ethyl}-carbamic acid benzyl ester as a mixture of epimers.

In a 25 ml round-bottomed flask, {(S)-1-[(S)-1-((S)-1-benzyl-2-cyclopropylcarbamoyl-2-hydroxy-ethylcarbamoyl)-2-(1H-indol-3-yl)-ethylcarbamoyl]-ethyl}-carbamic acid benzyl ester (65 mg, 0.10 mmol) was partially dissolved in 4.4 ml dichloromethane and Dess-Martin periodinane (66 mg, 0.16 mmol) was added. The reaction mixture was stirred at room temperature for 2.5 h (the reaction mixture turned into a dark brown solution). The reaction mixture was quenched with 1.5 ml saturated NaHCO₃-solution and 1.5 ml 10% Na₂S₂O₃-solution and stirred vigorously for 30 min at room temperature. The biphasic mixture was then extracted with 30 ml dichloromethane. The organic layer was washed with 3 ml saturated NaHCO₃-solution. The aqueous layers were extracted twice with 30 ml dichloromethane. The organic layers were combined, dried over sodium sulfate, filtered and concentrated. The residue was triturated with ethyl acetate/diethyl ether to afford 29 mg (43%) {(S)-1-[(S)-1-((S)-1-benzyl-2-cyclopropylcarbamoyl-2-oxo-ethylcarbamoyl)-2-(1H-indol-3-yl)-ethylcarbamoyl]-ethyl}-carbamic acid benzyl ester as a brown solid. LC/MS: (M+H)⁺=624.

### Example 33

### {(S)-1-[(S)-1-[(S)-1-Benzyl-2-(2-methoxy-ethylcarbamoyl)-2-oxo-ethylcarbamoyl]-2-(1H-indol-3-yl)-ethylcarbamoyl]-ethyl}-carbamic acid benzyl ester

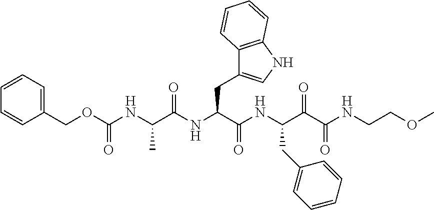 [SMILES: COCCNC(=O)C(=O)[C@H](Cc1ccccc1)NC(=O)[C@H](Cc1c[nH]c2ccccc12)NC(=O)[C@H](C)NC(=O)OCc1ccccc1]

A 10 ml round-bottomed flask was charged with S)-3-tert-butoxycarbonylamino-2-hydroxy-4-phenyl-butyric acid (200 mg, 0.64 mmol), 2-methoxyethylamine (86.4 mg, 0.10 ml, 1.15 mmol) and 2.5 ml dichloromethane. N,N-Diisopropylethylamine (170 mg, 0.23 ml, 1.32 mmol) was added followed by HATU (269 mg, 0.71 mmol). The yellow solution was stirred at room temperature overnight. The reaction mixture was extracted with 5 ml water and 30 ml dichloromethane. The aqueous layers were extracted with 30 ml dichloromethane. The organic layers were combined, dried over sodium sulfate, filtered and concentrated. The residue was chromatographed over 12 g silica gel with EtOAc/Hexanes (gradient 0-70% EtOAc). All fractions containing product were combined and concentrated to afford 75 mg (30%; purity=90%) [(S)-1-benzyl-2-hydroxy-2-(2-methoxy-ethylcarbamoyl)-ethyl]-carbamic acid tert-butyl ester as a light yellow oil and as a mixture of epimers.

In a 10 ml round-bottomed flask, [(S)-1-benzyl-2-hydroxy-2-(2-methoxy-ethylcarbamoyl)-ethyl]-carbamic acid tert-butyl ester (70 mg, 0.18 mmol; purity=90%) was dissolved in 1.5 ml dichloromethane. Trifluoroacetic acid (0.36 ml, 4.67 mmol) was added slowly. The reaction mixture was stirred at room temperature for 2.5 h. The solvent was evaporated and then placed under high vacuum for 15 min. The residue was dissolved in 1.0 ml DMF and cooled to 0° C. N,N-Diisopropylethylamine (118 mg, 0.16 ml, 0.92 mmol) was added dropwise at 0° C. followed by (S)-2-((S)-2-benzyloxycarbonylamino-propionylamino)-3-(1H-indol-3-yl)-propionic acid (80 mg, 0.16 mmol; purity=80%) and HATU (66 mg, 0.17 mmol). After the addition was complete, the ice bath was removed and the reaction mixture was stirred at room temperature overnight. The reaction mixture was extracted with EtOAc and water. The aqueous layer was back extracted with EtOAc. The organic layers were washed twice with water and once with brine. The organic layers were combined, dried over sodium sulfate, filtered and concentrated. The residue was absorbed on 1 g silica gel and chromatographed over 4 g silica gel with MeOH/Dichloromethane (gradient 0-10% MeOH). All fractions containing product were combined and concentrated to afford 67 mg (67%) {(S)-1-[(S)-1-[(S)-1-benzyl-2-hydroxy-2-(2-methoxy-ethylcarbamoyl)-ethylcarbamoyl]-2-(1H-indol-3-yl)-ethylcarbamoyl]-ethyl}-carbamic acid benzyl ester as a light yellow solid and as a mixture of epimers.

In a 25 ml round-bottomed flask, {(S)-1-[(S)-1-[(S)-1-benzyl-2-hydroxy-2-(2-methoxy-ethylcarbamoyl)-ethylcarbamoyl]-2-(1H-indol-3-yl)-ethylcarbamoyl]-ethyl}-carbamic acid benzyl ester (64 mg, 0.10 mmol) was dissolved in 4.2 ml dichloromethane and Dess-Martin periodinane (64 mg, 0.15 mmol) was added. The reaction mixture was stirred at room temperature for 2 h. The reaction mixture was quenched with 1.5 ml saturated NaHCO₃-solution and 1.5 ml 10% Na₂S₂O₃-solution and stirred vigorously for 30 min at room temperature. The biphasic mixture was then extracted with 30 ml dichloromethane. The organic layer was washed with 3 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 4 g silica gel with MeOH/dichloromethane (gradient 0-10% MeOH). All fractions containing product were combined and concentrated. The residue was triturated with ethyl acetate/diethyl ether and dichloromethane/hexanes to afford 14 mg (20%; purity=90%) {(S)-1-[(S)-1-[(S)-1-benzyl-2-(2-methoxy-ethylcarbamoyl)-2-oxo-ethylcarbamoyl]-2-(1H-indol-3-yl)-ethylcarbamoyl]-ethyl}-carbamic acid benzyl ester as a light brown solid. LC/MS: (M+H)⁺=642.

### Example 34

### {(S)-1-[(S)-1-{(S)-1-Benzyl-2-oxo-2-[(pyridin-2-ylmethyl)-carbamoyl]-ethylcarbamoyl}-2-(1H-indol-3-yl)-ethylcarbamoyl]-ethyl}-carbamic acid benzyl ester

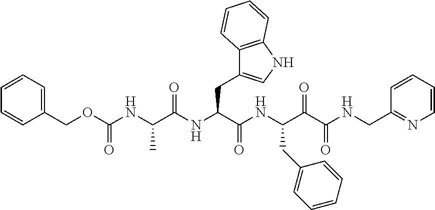 [SMILES: C[C@H](NC(=O)OCc1ccccc1)C(=O)N[C@@H](Cc1c[nH]c2ccccc12)C(=O)N[C@@H](Cc1ccccc1)C(=O)C(=O)NCc1ccccn1]

A 10 ml round-bottomed flask was charged with (S)-3-tert-butoxycarbonylamino-2-hydroxy-4-phenyl-butyric acid (220 mg, 0.71 mmol), 2-(aminomethyl)pyridine (157 mg, 0.15 ml, 1.46 mmol) and 2.5 ml DMF. N,N-Diisopropylethylamine (185 mg, 0.25 ml, 1.43 mmol) was added followed by HATU (296 mg, 0.78 mmol). The yellow solution was stirred at room temperature for four days. The reaction mixture was extracted with 50 ml EtOAc and 5 ml water. The aqueous layer was extracted with 50 ml EtOAc. The organic layers were washed three times with 5 ml water and once with 5 ml brine. The organic layers were combined, dried over sodium sulfate, filtered and concentrated. The residue was chromatographed over 12 g silica gel with EtOAc/hexanes (gradient 50-100% EtOAc). All fractions containing product were combined and concentrated to afford 107 mg (31%; purity=80%) as a light yellow waxy solid and as a mixture of epimers.

In a 25 ml round-bottomed flask, {(S)-1-benzyl-2-hydroxy-2-[(pyridin-2-ylmethyl)-carbamoyl]-ethyl}-carbamic acid tert-butyl ester (105 mg, 0.22 mmol; purity=80%) was dissolved in 2.1 ml dichloromethane. Trifluoroacetic acid (0.42 ml, 5.45 mmol) was added slowly. The reaction mixture was stirred at room temperature for 2.5 h. The solvent was evaporated and then placed under high vacuum for 30 min. The residue (light yellow oil) was dissolved in 1.4 ml DMF and cooled to 0° C. N,N-Diisopropylethylamine (148 mg, 0.20 ml, 1.15 mmol) was added dropwise at 0° C. (S)-2-((S)-2-benzyloxycarbonylamino-propionylamino)-3-(1H-indol-3-yl)-propionic acid (95 mg, 0.19 mmol; purity=80%) was added followed by HATU (78 mg, 0.21 mmol). After the addition was complete, the ice bath was removed and the reaction mixture was stirred at room temperature overnight. The reaction mixture was diluted with water (a precipitate was formed). The suspension was filtered, rinsing with water and a little petroleum ether. The resulting light yellow solid was dried using the rotavap and then placed under high vacuum to provide 112 mg (89%) {(S)-1-[(S)-1-{(S)-1-Benzyl-2-hydroxy-2-[(pyridin-2-ylmethyl)-carbamoyl]-ethylcarbamoyl}-2-(1H-indol-3-yl)-ethylcarbamoyl]-ethyl}-carbamic acid benzyl ester as a mixture of epimers.

In a 25 ml round-bottomed flask, {(S)-1-[(S)-1-{(S)-1-benzyl-2-hydroxy-2-[(pyridin-2-ylmethyl)-carbamoyl]-ethylcarbamoyl}-2-(1H-indol-3-yl)-ethylcarbamoyl]-ethyl}-carbamic acid benzyl ester (107 mg, 0.16 mmol) was partially dissolved in 7.0 ml dichloromethane and Dess-Martin periodinane (101 mg, 0.24 mmol) was added. The reaction mixture was stirred at room temperature for 2 h. The reaction mixture was quenched with 3 ml saturated NaHCO₃-solution and 3 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated to afford 22 mg (19%; purity=90%) {(S)-1-[(S)-1-{(S)-1-Benzyl-2-oxo-2-[(pyridin-2-ylmethyl)-carbamoyl]-ethylcarbamoyl}-2-(1H-indol-3-yl)-ethylcarbamoyl]-ethyl}-carbamic acid benzyl ester as a light brown solid. LC/MS: (M+H)⁺=675.

### Example 35

### (S)—N-Benzyl-3-[(S)-2-[(S)-2-(2-2,3-dihydro-benzo[1,4]oxazin-4-yl-acetylamino)-propionylamino]-3-(4-methoxy-phenyl)-propionylamino]-2-oxo-4-phenyl-butyramide

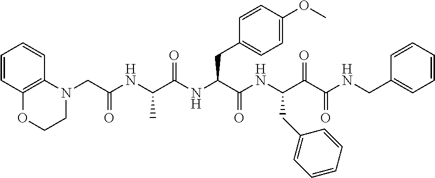 [SMILES: COc1ccc(C[C@H](NC(=O)[C@H](C)NC(=O)CN2CCOc3ccccc32)C(=O)N[C@@H](Cc2ccccc2)C(=O)C(=O)NCc2ccccc2)cc1]

To a solution of 3,4-dihydro-2H-benzo[b][1,4]oxazine (500 mg, 3.7 mmol) in DMF (12 ml) at room temperature was added potassium carbonate (1.02 g, 7.4 mmol) followed by methyl 2-bromoacetate (622 mg, 0.38 ml, 4.07 mmol). The reaction mixture was stirred at room temperature for 30 min then heated at 50° C. for 3 hr. Cooled to room temperature overnight. Quenched with water and extracted with Et₂O/EtOAc (2×). The combined organic layers were washed with water (3×) and brine then dried over MgSO₄ and concentrated. The residue was purified by chromatography over 40 g silica gel with 10% to 25% EtOAc/hexanes to afford 545 mg (71%) of (2,3-dihydro-benzo[1,4]oxazin-4-yl)-acetic acid methyl ester as an orange oil.

To a solution of (2,3-dihydro-benzo[1,4]oxazin-4-yl)-acetic acid methyl ester (545 mg, 2.63 mmol) in THF (4 ml), MeOH (4 ml), and water (2 ml) was added lithium hydroxide monohydrate (166 mg, 3.94 mmol). The orange reaction mixture was stirred at room temperature overnight then concentrated under reduced pressure. The orange aqueous residue which remained was acidified with 1.0M HCl. The resultant light brown precipitate was collected via filtration, rinsed with water and dried under high vacuum to afford 323 mg (64%) of (2,3-dihydro-benzo[1,4]oxazin-4-yl)-acetic acid.

In a 25 ml round-bottomed flask, (S)-2-((S)-2-tert-butoxycarbonylamino-propionylamino)-3-(4-methoxy-phenyl)-propionic acid benzyl ester (557 mg, 1.16 mmol) was dissolved in 6.0 ml dichloromethane. Trifluoroacetic acid (2.3 ml, 29.9 mmol) was added slowly. The reaction mixture was stirred at room temperature for 2.5 h. The solvent was evaporated and then put under high vacuum for 15 min. The residue (light brown solid) was dissolved in 4.0 ml DMF and cooled to 0° C. N,N-Diisopropylethylamine (814 mg, 1.1 ml, 6.3 mmol) was added dropwise at 0° C. (2,3-Dihydro-benzo[1,4]oxazin-4-yl)-acetic acid (200 mg, 1.04 mmol) was added followed by HATU (433 mg, 1.14 mmol). After the addition was complete, the ice bath was removed and the reaction mixture was stirred at room temperature overnight. The reaction mixture was diluted with water (a precipitate was formed). The suspension was filtered, rinsing with water and a little petroleum ether. The resulting off-white solid was dried using the rotavap and then placed under high vacuum to provide 508 mg (92%) (S)-2-[(S)-2-(2-2,3-dihydro-benzo[1,4]oxazin-4-yl-acetylamino)-propionylamino]-3-(4-methoxy-phenyl)-propionic acid benzyl ester.

In a 100 ml round-bottomed flask, (S)-2-[(S)-2-(2-2,3-dihydro-benzo[1,4]oxazin-4-yl-acetylamino)-propionylamino]-3-(4-methoxy-phenyl)-propionic acid benzyl ester (505 mg, 0.95 mmol) was dissolved in 6.0 ml methanol and 6.0 ml THF. The flask was three times alternating evacuated and flushed with argon. 20% Palladium hydroxide on carbon (wet, 86 mg, 0.12 mmol) was added carefully. The flask was evacuated, flushed with argon, evacuated and flushed with hydrogen. The reaction mixture was stirred under hydrogen atmosphere (balloon) at room temperature for 2 h. The reaction mixture was filtered over Celite, rinsing with ethyl acetate/methanol. The filtrate was concentrated to afford 464 mg (99%; purity=90%) (S)-2-[(S)-2-(2-2,3-dihydro-benzo[1,4]oxazin-4-yl-acetylamino)-propionylamino]-3-(4-methoxy-phenyl)-propionic acid as a light brown solid.

In a 10 ml round-bottomed flask, ((S)-1-benzyl-2-benzylcarbamoyl-2-hydroxy-ethyl)-carbamic acid tert-butyl ester (130 mg, 0.34 mmol) was dissolved in 3.0 ml dichloromethane. Trifluoroacetic acid (0.68 ml, 8.83 mmol) was added slowly. The reaction mixture was stirred at room temperature for 3.5 h. The solvent was evaporated and then placed under high vacuum for 15 min. The residue (light brown oil) was dissolved in 2.0 ml DMF and cooled to 0° C. N,N-Diisopropylethylamine (222 mg, 0.30 ml, 1.72 mmol) was added dropwise at 0° C. (S)-2-[(S)-2-(2-2,3-dihydro-benzo[1,4]oxazin-4-yl-acetylamino)-propionylamino]-3-(4-methoxy-phenyl)-propionic acid (140 mg, 0.29 mmol; purity=90%) was added followed by HATU (119 mg, 0.31 mmol). After the addition was complete, the ice bath was removed and the reaction mixture was stirred at room temperature overnight. The reaction mixture was diluted with water (a precipitate was formed). The suspension was filtered, rinsing with water and a little petroleum ether. The resulting light brown solid was dried using the rotavap and then placed under high vacuum to afford 195 mg (97%) (S)—N-benzyl-3-[(S)-2-[(S)-2-(2-2,3-dihydro-benzo[1,4]oxazin-4-yl-acetylamino)-propionylamino]-3-(4-methoxy-phenyl)-propionylamino]-2-hydroxy-4-phenyl-butyramide as a mixture of epimers.

In a 50 ml round-bottomed flask, (S)—N-benzyl-3-[(S)-2-[(S)-2-(2-2,3-dihydro-benzo[1,4]oxazin-4-yl-acetylamino)-propionylamino]-3-(4-methoxy-phenyl)-propionylamino]-2-hydroxy-4-phenyl-butyramide (184 mg, 0.26 mmol) was partially dissolved in 11 ml dichloromethane and Dess-Martin periodinane (165 mg, 0.39 mmol) was added. The reaction mixture was stirred at room temperature for 1.5 h. The reaction mixture was quenched with 4.5 ml saturated NaHCO₃-solution and 4.5 ml 10% Na₂S₂O₃-solution and stirred vigorously for 15 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was absorbed on 1 g silica gel and chromatographed over 12 g silica gel with MeOH/dichloromethane (gradient 0-5% MeOH). All fractions containing product were combined and concentrated. The residue was triturated with ethyl acetate/diethyl ether and a few drops of dichloromethane to afford 54 mg (26%; purity=90%) (S)—N-benzyl-3-[(S)-2-[(S)-2-(2-2,3-dihydro-benzo[1,4]oxazin-4-yl-acetylamino)-propionylamino]-3-(4-methoxy-phenyl)-propionylamino]-2-oxo-4-phenyl-butyramide as an orange solid. LC/MS: (M−H)⁻=704.

### Example 36

### {(S)-1-[(S)-1-((S)-1-Benzyl-2-benzylcarbamoyl-2-oxo-ethylcarbamoyl)-3-methyl-butylcarbamoyl]-ethyl}-carbamic acid benzyl ester

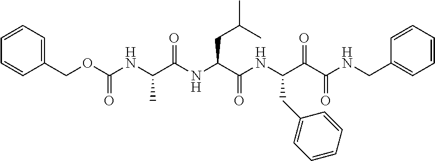 [SMILES: CC(C)C[C@H](NC(=O)[C@H](C)NC(=O)OCc1ccccc1)C(=O)N[C@@H](Cc1ccccc1)C(=O)C(=O)NCc1ccccc1]

In a 50 ml round-bottomed flask, L-leucine tert-butyl ester hydrochloride (662 mg, 2.96 mmol) was dissolved in 10 ml DMF and cooled to 0° C. N,N-Diisopropylethylamine (1.04 g, 1.4 ml, 8.02 mmol) was added dropwise at 0° C. N-Benzyloxycarbonyl-L-alanine (600 mg, 2.69 mmol) was added followed by HATU (1.12 g, 2.96 mmol). After the addition was complete, the ice bath was removed and the reaction mixture was stirred at room temperature overnight. The reaction mixture was extracted with 100 ml diethyl ether and 10 ml water. The aqueous layer was extracted with 100 ml diethyl ether. The organic layers were washed three times with 10 ml water and once with 10 ml brine. The organic layers were combined, dried over sodium sulfate, filtered and concentrated to afford (S)-2-((S)-2-benzyloxycarbonylamino-propionylamino)-4-methyl-pentanoic acid tert-butyl ester as a light yellow oil which was used without further purification.

A microwave vial was charged with (S)-2-((S)-2-benzyloxycarbonylamino-propionylamino)-4-methyl-pentanoic acid tert-butyl ester (125 mg, 0.27 mmol; purity=85%) and 1,1,1,3,3,3-hexafluoro-2-propanol (2.2 ml, 20.9 mmol). The vial was flushed with argon and sealed. The colorless solution was heated at 120° C. for 2 h under microwave irradiation. The reaction mixture was concentrated to provide (S)-2-((S)-2-benzyloxycarbonylamino-propionylamino)-4-methyl-pentanoic acid as a colorless oil which was used without further purification.

In a 10 ml round-bottomed flask, ((S)-1-benzyl-2-benzylcarbamoyl-2-hydroxy-ethyl)-carbamic acid tert-butyl ester (125 mg, 0.33 mmol) was dissolved in 3.0 ml dichloromethane. Trifluoroacetic acid (0.60 ml, 7.79 mmol) was added slowly. The reaction mixture was stirred at room temperature for 3 h. The solvent was evaporated and then placed under high vacuum for 15 min. The residue and (S)-2-((S)-2-benzyloxycarbonylamino-propionylamino)-4-methyl-pentanoic acid (111 mg, 0.23 mmol; purity=70%) were dissolved in 2.0 ml DMF and cooled to 0° C. N,N-Diisopropylethylamine (222 mg, 0.30 ml, 1.72 mmol) was added dropwise at 0° followed by HATU (97 mg, 0.26 mmol). After the addition was complete, the ice bath was removed and the reaction mixture was stirred at room temperature overnight. The reaction mixture was diluted with water (a precipitate was formed). The suspension was filtered, rinsing with water and a little petroleum ether. The resulting off-white solid was dried using the rotavap and then placed under high vacuum to afford 123 mg (80%; purity=90%) {(S)-1-[(S)-1-((S)-1-benzyl-2-benzylcarbamoyl-2-hydroxy-ethylcarbamoyl)-3-methyl-butylcarbamoyl]-ethyl}-carbamic acid benzyl ester as a mixture of epimers.

In a 25 ml round-bottomed flask, {(S)-1-[(S)-1-((S)-1-benzyl-2-benzylcarbamoyl-2-hydroxy-ethylcarbamoyl)-3-methyl-butylcarbamoyl]-ethyl}-carbamic acid benzyl ester (117 mg, 0.17 mmol; purity=90%) was partially dissolved in 7.0 ml dichloromethane and Dess-Martin periodinane (111 mg, 0.26 mmol) was added. The reaction mixture was stirred at room temperature for 2 h. The reaction mixture was quenched with 3.5 ml saturated NaHCO₃-solution and 3.5 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was triturated with ethyl acetate/diethyl ether to afford 71 mg (64%) {(S)-1-[(S)-1-((S)-1-benzyl-2-benzylcarbamoyl-2-oxo-ethylcarbamoyl)-3-methyl-butylcarbamoyl]-ethyl}-carbamic acid benzyl ester as a white solid. LC/MS: (M+H)⁺=601.

### Example 37

### 2-Methyl-2H-pyrazole-3-carboxylic acid {(S)-1-[(S)-1-((S)-1-benzyl-2-benzylcarbamoyl-2-oxo-ethylcarbamoyl)-3-methyl-butylcarbamoyl]-ethyl}-amide

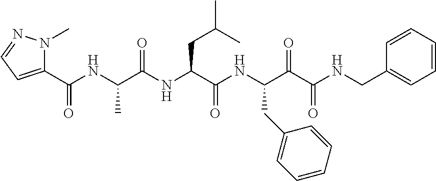 [SMILES: CC(C)C[C@H](NC(=O)[C@H](C)NC(=O)c1ccnn1C)C(=O)N[C@@H](Cc1ccccc1)C(=O)C(=O)NCc1ccccc1]

In a 50 ml round-bottomed flask, (S)-2-((S)-2-tert-butoxycarbonylamino-propionylamino)-4-methyl-pentanoic acid benzyl ester (400 mg, 0.97 mmol) was dissolved in 6.0 ml dichloromethane. Trifluoroacetic acid (2.0 ml, 26.0 mmol) was added slowly. The reaction mixture was stirred at room temperature for 2.5 h. The solvent was evaporated and then placed under high vacuum for 15 min. The residue (colorless oil) was dissolved in 4.0 ml DMF and cooled to 0° C. N,N-Diisopropylethylamine (1.48 g, 2.0 ml, 11.5 mmol) was added dropwise at 0° C. 1-Methyl-1H-pyrazole-5-carboxylic acid (115 mg, 0.91 mmol) was added followed by HATU (381 mg, 1.00 mmol). After the addition was complete, the ice bath was removed and the reaction mixture was stirred at room temperature overnight. The reaction mixture was extracted with 70 ml diethyl ether and 5 ml water. The aqueous layer was extracted with 70 ml diethyl ether. The organic layers were washed three times with 5 ml water and once with 5 ml brine. The organic layers were combined, dried over sodium sulfate, filtered and concentrated. The residue was chromatographed over 12 g silica gel with EtOAc/hexanes (gradient 0-70% EtOAc). All fractions containing product were combined and concentrated to afford 382 mg (99%; purity=95%) (S)-4-methyl-2-{(S)-2-[(2-methyl-2H-pyrazole-3-carbonyl)-amino]-propionylamino}-pentanoic acid benzyl ester as a colorless oil.

In a 50 ml round-bottomed flask, (S)-4-methyl-2-{(S)-2-[(2-methyl-2H-pyrazole-3-carbonyl)-amino]-propionylamino}-pentanoic acid benzyl ester (380 mg, 0.90 mmol) was dissolved in 9.0 ml methanol. The flask was three times alternating evacuated and flushed with argon. 20% Palladium hydroxide on carbon (wet, 82 mg, 0.12 mmol) was added carefully. The flask was evacuated, flushed with argon, evacuated and flushed with hydrogen. The reaction mixture was stirred under hydrogen atmosphere (balloon) at room temperature for 2 h. The reaction mixture was filtered over Celite, rinsing with ethyl acetate/methanol. The filtrate was concentrated to afford 292 mg (94%; purity=90%) (S)-4-methyl-2-{(S)-2-[(2-methyl-2H-pyrazole-3-carbonyl)-amino]-propionylamino}-pentanoic acid as a white solid.

In a 10 ml round-bottomed flask, ((S)-1-benzyl-2-benzylcarbamoyl-2-hydroxy-ethyl)-carbamic acid tert-butyl ester (125 mg, 0.33 mmol) was dissolved in 3.0 ml dichloromethane. Trifluoroacetic acid (0.60 ml, 7.79 mmol) was added slowly. The reaction mixture was stirred at room temperature for 2.5 h. The solvent was evaporated and then placed under high vacuum for 15 min. The residue and (S)-4-methyl-2-{(S)-2-[(2-methyl-2H-pyrazole-3-carbonyl)-amino]-propionylamino}-pentanoic acid (100 mg, 0.29 mmol; purity=90%) were dissolved in 2.0 ml DMF and cooled to 0° C. N,N-Diisopropylethylamine (259 mg, 0.35 ml, 2.00 mmol) was added dropwise at 0° followed by HATU (121 mg, 0.32 mmol). After the addition was complete, the ice bath was removed and the reaction mixture was stirred at room temperature overnight. The reaction mixture was diluted with water (a precipitate was formed). The suspension was filtered, rinsing with water and a little petroleum ether. The resulting off-white solid was dried using the rotavap and then placed under high vacuum to afford 146 mg (87%) 2-methyl-2H-pyrazole-3-carboxylic acid {(S)-1-[(S)-1-((S)-1-benzyl-2-benzylcarbamoyl-2-hydroxy-ethylcarbamoyl)-3-methyl-butylcarbamoyl]-ethyl}-amide as a mixture of epimers.

In a 25 ml round-bottomed flask, 2-methyl-2H-pyrazole-3-carboxylic acid {(S)-1-[(S)-1-((S)-1-benzyl-2-benzylcarbamoyl-2-hydroxy-ethylcarbamoyl)-3-methyl-butylcarbamoyl]-ethyl}-amide (141 mg, 0.24 mmol) was partially dissolved in 10 ml dichloromethane and Dess-Martin periodinane (156 mg, 0.37 mmol) was added. The reaction mixture was stirred at room temperature for 2 h. The reaction mixture was quenched with 4 ml saturated NaHCO₃-solution and 4 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was triturated with dichloromethane/diethyl ether to afford 111 mg (75%) 2-methyl-2H-pyrazole-3-carboxylic acid {(S)-1-[(S)-1-((S)-1-benzyl-2-benzylcarbamoyl-2-oxo-ethylcarbamoyl)-3-methyl-butylcarbamoyl]-ethyl}-amide as an off-white solid. LC/MS: (M+H)⁺=575.

### Example 38

### {(S)-1-[(S)-1-[(S)-1-Benzyl-2-(benzyl-methyl-carbamoyl)-2-oxo-ethylcarbamoyl]-2-(1H-indol-3-yl)-ethylcarbamoyl]-ethyl}-carbamic acid benzyl ester

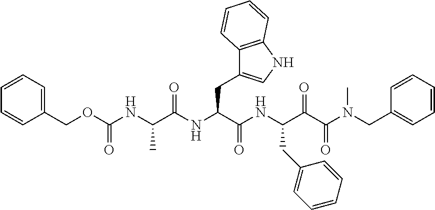 [SMILES: C[C@H](NC(=O)OCc1ccccc1)C(=O)N[C@@H](Cc1c[nH]c2ccccc12)C(=O)N[C@@H](Cc1ccccc1)C(=O)C(=O)N(C)Cc1ccccc1]

A 10 ml round-bottomed flask was charged with (S)-3-tert-butoxycarbonylamino-2-hydroxy-4-phenyl-butyric acid (220 mg, 0.71 mmol), N-benzylmethylamine (170 mg, 0.18 ml, 1.4 mmol) and 2.5 ml DMF. N,N-Diisopropylethylamine (185 mg, 0.25 ml, 1.43 mmol) was added followed by HATU (296 mg, 0.78 mmol). The yellow solution was stirred at room temperature overnight. The reaction mixture was extracted with 50 ml EtOAc and 5 ml water. The aqueous layer was extracted with 50 ml EtOAc. The organic layers were washed three times with 5 ml water and once with 5 ml brine. The organic layers were combined, dried over sodium sulfate, filtered and concentrated. The residue was chromatographed over 12 g silica gel with EtOAc/hexanes (gradient 0-30% EtOAc). All fractions containing product were combined and concentrated to afford 213 mg (76%) [(S)-1-benzyl-2-(benzyl-methyl-carbamoyl)-2-hydroxy-ethyl]-carbamic acid tert-butyl ester as a light yellow oil and as a mixture of epimers.

In a 25 ml round-bottomed flask, [(S)-1-benzyl-2-(benzyl-methyl-carbamoyl)-2-hydroxy-ethyl]-carbamic acid tert-butyl ester (209 mg, 0.52 mmol) was dissolved in 3.0 ml dichloromethane. Trifluoroacetic acid (0.81 ml, 10.5 mmol) was added slowly. The reaction mixture was stirred at room temperature for 2.5 h. The solvent was evaporated and then placed under high vacuum for 15 min. The residue was dissolved in 2.0 ml DMF and cooled to 0° C. N,N-Diisopropylethylamine (274 mg, 0.37 ml, 2.12 mmol) was added dropwise at 0° C. (S)-2-((S)-2-Benzyloxycarbonylamino-propionylamino)-3-(1H-indol-3-yl)-propionic acid (180 mg, 0.35 mmol; purity=80%) was added followed by HATU (147 mg, 0.39 mmol). After the addition was complete, the ice bath was removed and the reaction mixture was stirred at room temperature for 2 d. The reaction mixture was extracted with 40 ml diethyl ether and 4 ml water. The aqueous layer was extracted with 40 ml diethyl ether. The organic layers were washed twice with 4 ml water and once with 4 ml brine. The organic layers were combined, dried over sodium sulfate, filtered and concentrated. The residue was chromatographed over 12 g silica gel with MeOH/dichloromethane (gradient 0-5% MeOH). All fractions containing product were combined and concentrated to afford 212 mg (87%) {(S)-1-[(S)-1-[(S)-1-benzyl-2-(benzyl-methyl-carbamoyl)-2-hydroxy-ethylcarbamoyl]-2-(1H-indol-3-yl)-ethylcarbamoyl]-ethyl}-carbamic acid benzyl ester as a light yellow solid and as a mixture of epimers.

In a 25 ml round-bottomed flask, {(S)-1-[(S)-1-[(S)-1-benzyl-2-(benzyl-methyl-carbamoyl)-2-hydroxy-ethylcarbamoyl]-2-(1H-indol-3-yl)-ethylcarbamoyl]-ethyl}-carbamic acid benzyl ester (205 mg, 0.30 mmol) was dissolved in 12 ml dichloromethane and Dess-Martin periodinane (189 mg, 0.45 mmol) was added. The reaction mixture was stirred at room temperature for 3.5 h. The reaction mixture was quenched with 4.5 ml saturated NaHCO₃-solution and 4.5 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was chromatographed over 12 g silica gel with MeOH/dichloromethane (gradient 0-5% MeOH). All fractions containing product were combined and concentrated to afford 29 mg (13%; purity=90%) {(S)-1-[(S)-1-[(S)-1-benzyl-2-(benzyl-methyl-carbamoyl)-2-oxo-ethylcarbamoyl]-2-(1H-indol-3-yl)-ethylcarbamoyl]-ethyl}-carbamic acid benzyl ester as a light brown foam. LC/MS: (M-OBn)⁻=578.

### Example 39

### (S)-3-[(S)-2-((S)-2-Benzenesulfonylamino-propionylamino)-3-(4-methoxy-phenyl)-propionylamino]-N-benzyl-2-oxo-4-phenyl-butyramide

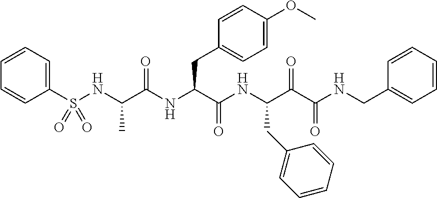 [SMILES: COc1ccc(C[C@H](NC(=O)[C@H](C)NS(=O)(=O)c2ccccc2)C(=O)N[C@@H](Cc2ccccc2)C(=O)C(=O)NCc2ccccc2)cc1]

In a 50 ml round-bottomed flask, (S)-2-((S)-2-tert-butoxycarbonylamino-propionylamino)-3-(4-methoxy-phenyl)-propionic acid benzyl ester (400 mg, 0.70 mmol; purity=80%) was dissolved in 6.0 ml dichloromethane. Trifluoroacetic acid (1.3 ml, 16.9 mmol) was added slowly. The reaction mixture was stirred at room temperature for 2.5 h. The solvent was evaporated and then placed under high vacuum for 30 min. The residue (off-white solid) was partially dissolved in 4.8 ml dichloromethane and cooled to 0° C. N,N-Diisopropylethylamine (459 mg, 0.62 ml, 3.55 mmol) was added at 0° C. followed by benzenesulfonyl chloride (137 mg, 0.10 ml, 0.78 mmol). The reaction mixture was allowed to warm slowly to room temperature with stirring overnight. The reaction mixture was quenched with 5 ml water and extracted twice with 30 ml Dichloromethane. The organic layers were combined, dried over sodium sulfate, filtered and concentrated. The residue (brown oil) was chromatographed over 12 g silica gel with EtOAc/hexanes (gradient 0-40% EtOAc). All fractions containing product were combined and concentrated to afford 187 mg (51%) (S)-2-((S)-2-benzenesulfonylamino-propionylamino)-3-(4-methoxy-phenyl)-propionic acid benzyl ester as a brown oil.

In a 25 ml round-bottomed flask, (S)-2-((S)-2-benzenesulfonylamino-propionylamino)-3-(4-methoxy-phenyl)-propionic acid benzyl ester (184 mg, 0.35 mmol) was dissolved in 4.0 ml methanol. The flask was three times alternating evacuated and flushed with argon. 20% Palladium hydroxide on carbon (33 mg, 0.05 mmol) was added carefully. The flask was evacuated, flushed with argon, evacuated and flushed with hydrogen. The reaction mixture was stirred under hydrogen atmosphere (balloon) at room temperature for 2 h. The reaction mixture was filtered over Celite, rinsing with ethyl acetate/methanol. The filtrate was concentrated to afford 142 mg (94%; purity=95%) (S)-2-((S)-2-benzenesulfonylamino-propionylamino)-3-(4-methoxy-phenyl)-propionic acid as a light yellow foam.

In a 10 ml round-bottomed flask, ((S)-1-benzyl-2-benzylcarbamoyl-2-hydroxy-ethyl)-carbamic acid tert-butyl ester (125 mg, 0.33 mmol) was dissolved in 3.0 ml dichloromethane. Trifluoroacetic acid (0.60 ml, 7.79 mmol) was added slowly. The reaction mixture was stirred at room temperature for 2.5 h. The solvent was evaporated and then placed under high vacuum for 30 min. The residue and (S)-2-((S)-2-benzenesulfonylamino-propionylamino)-3-(4-methoxy-phenyl)-propionic acid (125 mg, 0.29 mmol) were dissolved in 2.0 ml DMF and cooled to 0° C. N,N-Diisopropylethylamine (222 mg, 0.30 ml, 1.72 mmol) was added dropwise at 0° followed by HATU (122 mg, 0.32 mmol). After the addition was complete, the ice bath was removed and the reaction mixture was stirred at room temperature overnight (a precipitate was formed). The reaction mixture was diluted with water. The suspension was filtered, rinsing with water and a little petroleum ether. The resulting light yellow solid was dissolved in dichloromethane/methanol, dried over sodium sulfate, filtered, concentrated and then placed under high vacuum to provide 157 mg (76%) (S)-3-[(S)-2-((S)-2-benzenesulfonylamino-propionylamino)-3-(4-methoxy-phenyl)-propionylamino]-N-benzyl-2-hydroxy-4-phenyl-butyramide as a mixture of epimers.

In a 50 ml round-bottomed flask, (S)-3-[(S)-2-((S)-2-benzenesulfonylamino-propionylamino)-3-(4-methoxy-phenyl)-propionylamino]-N-benzyl-2-hydroxy-4-phenyl-butyramide (153 mg, 0.22 mmol) was partially dissolved in 9.0 ml dichloromethane and Dess-Martin periodinane (137 mg, 0.32 mmol) was added. The reaction mixture was stirred at room temperature for 2 h. The reaction mixture was quenched with 4 ml saturated NaHCO₃-solution and 4 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue (light yellow solid) was triturated with dichloromethane/diethyl ether to afford 87 mg (60%) (S)-3-[(S)-2-((S)-2-benzenesulfonylamino-propionylamino)-3-(4-methoxy-phenyl)-propionylamino]-N-benzyl-2-oxo-4-phenyl-butyramide as a white solid. LC/MS: (M−H)⁻=669.

### Example 40

### (S)—N-Benzyl-3-{(S)-3-(4-methoxy-phenyl)-2-[(S)-2-(toluene-2-sulfonylamino)-propionylamino]-propionylamino}-2-oxo-4-phenyl-butyramide

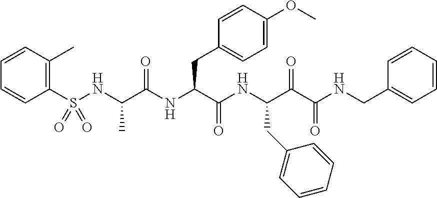 [SMILES: COc1ccc(C[C@H](NC(=O)[C@H](C)NS(=O)(=O)c2ccccc2C)C(=O)N[C@@H](Cc2ccccc2)C(=O)C(=O)NCc2ccccc2)cc1]

In a 50 ml round-bottomed flask, (S)-2-((S)-2-tert-butoxycarbonylamino-propionylamino)-3-(4-methoxy-phenyl)-propionic acid benzyl ester (400 mg, 0.70 mmol; purity=80%) was dissolved in 6.0 ml dichloromethane. Trifluoroacetic acid (1.3 ml, 16.9 mmol) was added slowly. The reaction mixture was stirred at room temperature for 2.5 h. The solvent was evaporated and then placed under high vacuum for 30 min. The residue (off-white solid) was partially dissolved in 4.8 ml dichloromethane and cooled to 0° C. N,N-Diisopropylethylamine (459 mg, 0.62 ml, 3.55 mmol) was added at 0° C. followed by o-toluenesulfonyl chloride (145 mg, 0.11 ml, 0.76 mmol). The reaction mixture was allowed to warm slowly to room temperature with stirring overnight. The reaction mixture was quenched with 5 ml water and extracted twice with 30 ml dichloromethane. The organic layers were combined, dried over sodium sulfate, filtered and concentrated. The residue (brown oil) was chromatographed over 12 g silica gel with EtOAc/hexanes (gradient 0-30% EtOAc). All fractions containing product were combined and concentrated to provide 212 mg (56%) (S)-3-(4-methoxy-phenyl)-2-[(S)-2-(toluene-2-sulfonylamino)-propionylamino]-propionic acid benzyl ester as a light brown oil.

In a 25 ml round-bottomed flask, (S)-3-(4-methoxy-phenyl)-2-[(S)-2-(toluene-2-sulfonylamino)-propionylamino]-propionic acid benzyl ester (210 mg, 0.39 mmol) was dissolved in 4.0 ml methanol. The flask was three times alternating evacuated and flushed with argon. 20% Palladium hydroxide on carbon (36 mg, 0.05 mmol) was added carefully. The flask was evacuated, flushed with argon, evacuated and flushed with hydrogen. The reaction mixture was stirred under hydrogen atmosphere (balloon) at room temperature for 2 h. The reaction mixture was filtered over Celite, rinsing with ethyl acetate/methanol. The filtrate was concentrated to afford 164 mg (95%; purity=95%) (S)-3-(4-methoxy-phenyl)-2-[(S)-2-(toluene-2-sulfonylamino)-propionylamino]-propionic acid as a light yellow foam.

In a 10 ml round-bottomed flask, ((S)-1-benzyl-2-benzylcarbamoyl-2-hydroxy-ethyl)-carbamic acid tert-butyl ester (125 mg, 0.33 mmol) was dissolved in 3.0 ml dichloromethane. Trifluoroacetic acid (0.60 ml, 7.79 mmol) was added slowly. The reaction mixture was stirred at room temperature for 3 h. The solvent was evaporated and then put under high vacuum for 30 min. The residue and (S)-3-(4-methoxy-phenyl)-2-[(S)-2-(toluene-2-sulfonylamino)-propionylamino]-propionic acid (130 mg, 0.29 mmol) were dissolved in 2.0 ml DMF and cooled to 0° C. N,N-Diisopropylethylamine (222 mg, 0.30 ml, 1.72 mmol) was added dropwise at 0° followed by HATU (123 mg, 0.32 mmol). After the addition was complete, the ice bath was removed and the reaction mixture was stirred at room temperature overnight. The reaction mixture was diluted with water (a precipitate was formed). The suspension was filtered, rinsing with water and a little petroleum ether. The resulting light brown solid was dissolved in dichloromethane/methanol, dried over sodium sulfate, filtered, concentrated and then placed under high vacuum to provide 175 mg (82%) (S)—N-benzyl-2-hydroxy-3-{(S)-3-(4-methoxy-phenyl)-2-[(S)-2-(toluene-2-sulfonylamino)-propionylamino]-propionylamino}-4-phenyl-butyramide as a mixture of epimers.

In a 50 ml round-bottomed flask, (S)—N-benzyl-2-hydroxy-3-{(S)-3-(4-methoxy-phenyl)-2-[(S)-2-(toluene-2-sulfonylamino)-propionylamino]-propionylamino}-4-phenyl-butyramide (170 mg, 0.24 mmol) was partially dissolved in 10.0 ml dichloromethane and Dess-Martin periodinane (150 mg, 0.35 mmol) was added. The reaction mixture was stirred at room temperature for 2 h. The reaction mixture was quenched with 4 ml saturated NaHCO₃-solution and 4 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue (light yellow solid) was triturated with dichloromethane/diethyl ether to afford 122 mg (72%) (S)—N-benzyl-3-{(S)-3-(4-methoxy-phenyl)-2-[(S)-2-(toluene-2-sulfonylamino)-propionylamino]-propionylamino}-2-oxo-4-phenyl-butyramide as a white solid. LC/MS: (M−H)⁻=683.

### Example 41

### (S)—N-Benzyl-3-((S)-3-(4-methoxy-phenyl)-2-{(S)-2-[3-(2-methyl-2H-pyrazol-3-yl)-propionylamino-]propionylamino}-propionylamino)-2-oxo-4-phenyl-butyramide

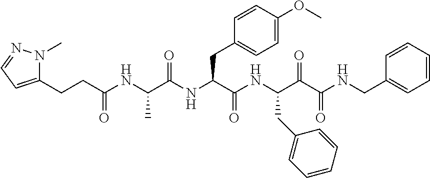 [SMILES: COc1ccc(C[C@H](NC(=O)[C@H](C)NC(=O)CCc2ccnn2C)C(=O)N[C@@H](Cc2ccccc2)C(=O)C(=O)NCc2ccccc2)cc1]

A 50 ml 2-neck round-bottomed flask was charged with 1-methyl-1H-pyrazole-5-boronic acid pinacol ester (1.15 g, 5.51 mmol), sodium bicarbonate (154 mg, 1.84 mmol), chloro(1,5-cyclooctadiene)rhodium(I) dimer (55 mg, 0.11 mmol), 12 ml 1,4-dioxane and 2.0 ml water. The reaction mixture was stirred at room temperature for 15 min then ethyl acrylate (368 mg, 0.40 ml, 3.68 mmol) was added. The reaction mixture was stirred at 80° C. for 2 h then cooled to room temperature and extracted with 70 ml EtOAc and 10 ml water. The aqueous layer was extracted with 70 ml EtOAc. The organic layers were washed with 10 ml water and 10 ml brine. The organic layers were combined, dried over sodium sulfate, filtered and concentrated. The residue was chromatographed over 25 g silica gel with EtOAc/hexanes (gradient 0-40% EtOAc). All fractions containing product were combined and concentrated to afford 314 mg (47%) 3-(2-methyl-2H-pyrazol-3-yl)-propionic acid ethyl ester as a yellow oil.

In a 25 ml round-bottomed flask, 3-(2-methyl-2H-pyrazol-3-yl)-propionic acid ethyl ester (313 mg, 1.72 mmol) was dissolved in 3.6 ml THF and 3.6 ml methanol. Lithium hydroxide (185 mg, 7.73 mmol) was added followed by 3.6 ml water. The yellow suspension was stirred at room temperature for 2 d. The organic solvents were evaporated and the aqueous layer was extracted with 20 ml dichloromethane. The organic layer was set aside and later discarded. The aqueous layer was acidified with 1M HCl to pH=4 and then extracted twice with 40 ml EtOAc. The organic layers were washed with 3 ml water and 3 ml brine then combined, dried over sodium sulfate, filtered and concentrated to afford 149 mg (56%) 3-(2-methyl-2H-pyrazol-3-yl)-propionic acid as a yellow solid.

In a 50 ml round-bottomed flask, (S)-2-((S)-2-tert-butoxycarbonylamino-propionylamino)-3-(4-methoxy-phenyl)-propionic acid benzyl ester (620 mg, 1.09 mmol; purity=80%) was dissolved in 6.0 ml dichloromethane. Trifluoroacetic acid (2.0 ml, 26.0 mmol) was added slowly. The reaction mixture was stirred at room temperature for 2.5 h. The solvent was evaporated and then placed under high vacuum for 15 min. The residue (off-white solid) and 3-(2-methyl-2H-pyrazol-3-yl)-propionic acid (147 mg, 0.95 mmol) were dissolved in 4.0 ml DMF. The yellow solution was cooled to 0° C. N,N-Diisopropylethylamine (740 mg, 1.0 ml, 5.73 mmol) was added dropwise at 0° C. followed by HATU (399 mg, 1.05 mmol). After the addition was complete, the ice bath was removed and the yellow solution was stirred at room temperature overnight. The reaction mixture was extracted with 70 ml diethyl ether and 5 ml water. The aqueous layer was extracted with 70 ml diethyl ether. The organic layers were washed twice with 5 ml water and once with 5 ml brine. The organic layers were combined, dried over sodium sulfate, filtered and concentrated. The residue (light yellow oil) was chromatographed over 25 g silica gel with EtOAc/hexanes (gradient 0-100% EtOAc) and then EtOAc/MeOH 19:1. All fractions containing product were combined and concentrated to provide 223 mg (47%) (S)-3-(4-methoxy-phenyl)-2-{(S)-2-[3-(2-methyl-2H-pyrazol-3-yl)-propionylamino]-propionylamino}-propionic acid benzyl ester as an off-white solid.

In a 50 ml round-bottomed flask, (S)-3-(4-methoxy-phenyl)-2-{(S)-2-[3-(2-methyl-2H-pyrazol-3-yl)-propionylamino]-propionylamino}-propionic acid benzyl ester (222 mg, 0.45 mmol) was dissolved in 5.0 ml methanol. The flask was three times alternating evacuated and flushed with argon. 20% Palladium hydroxide on carbon (42 mg, 0.06 mmol) was added carefully. The flask was evacuated, flushed with argon, evacuated and flushed with hydrogen. The reaction mixture was stirred under hydrogen atmosphere (balloon) at room temperature for 2 h (the product precipitated). The reaction mixture was filtered, rinsing with hot ethyl acetate/methanol. The filtrate was concentrated to afford 172 mg (95%) (S)-3-(4-methoxy-phenyl)-2-{(S)-2-[3-(2-methyl-2H-pyrazol-3-yl)-propionylamino]-propionylamino}-propionic acid as a white solid.

In a 10 ml round-bottomed flask, ((S)-1-benzyl-2-benzylcarbamoyl-2-hydroxy-ethyl)-carbamic acid tert-butyl ester (125 mg, 0.33 mmol) was dissolved in 3.0 ml dichloromethane. Trifluoroacetic acid (0.60 ml, 7.79 mmol) was added slowly. The reaction mixture was stirred at room temperature for 3.5 h. The solvent was evaporated and then placed under high vacuum for 30 min. The residue and (S)-3-(4-methoxy-phenyl)-2-{(S)-2-[3-(2-methyl-2H-pyrazol-3-yl)-propionylamino]-propionylamino}-propionic acid (115 mg, 0.29 mmol) were dissolved in 2.0 ml DMF and cooled to 0° C. N,N-Diisopropylethylamine (222 mg, 0.30 ml, 1.72 mmol) was added dropwise at 0° followed by HATU (120 mg, 0.31 mmol). After the addition was complete, the ice bath was removed and the reaction mixture was stirred at room temperature overnight (a precipitate was formed). The reaction mixture was diluted with water. The suspension was filtered, rinsing with water and a little petroleum ether. The resulting (wet) light yellow solid was dissolved in dichloromethane/methanol, dried over sodium sulfate, filtered, concentrated and then placed under high vacuum to afford 221 mg (98%; purity=85%) (S)—N-benzyl-2-hydroxy-3-((S)-3-(4-methoxy-phenyl)-2-{(S)-2-[3-(2-methyl-2H-pyrazol-3-yl)-propionylamino]-propionylamino}-propionylamino)-4-phenyl-butyramide as a mixture of epimers.

In a 50 ml round-bottomed flask, (S)—N-benzyl-2-hydroxy-3-((S)-3-(4-methoxy-phenyl)-2-{(S)-2-[3-(2-methyl-2H-pyrazol-3-yl)-propionylamino]-propionylamino}-propionylamino)-4-phenyl-butyramide (215 mg, 0.27 mmol; purity=85%) was partially dissolved in 12 ml dichloromethane and Dess-Martin periodinane (174 mg, 0.41 mmol) was added. The reaction mixture was stirred at room temperature for 2 h. The reaction mixture was quenched with 4.5 ml saturated NaHCO₃-solution and 4.5 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue (light yellow solid) was triturated with dichloromethane/diethyl ether/ethyl acetate. The residue (off-white solid) was absorbed on 1 g silica gel and chromatographed over 4 g silica gel with MeOH/Dichloromethane (gradient 0-10% MeOH). All fractions containing product were combined and concentrated to afford 41 mg (20%; purity=90%) (S)—N-benzyl-3-((S)-3-(4-methoxy-phenyl)-2-{(S)-2-[3-(2-methyl-2H-pyrazol-3-yl)-propionylamino]-propionylamino}-propionylamino)-2-oxo-4-phenyl-butyramide as a white solid. LC/MS: (M−H)⁻=665.

### Example 42

### Indan-2-carboxylic acid {(S)-1-[(S)-1-((S)-1-benzyl-2-benzylcarbamoyl-2-oxo-ethylcarbamoyl)-3-phenyl-propylcarbamoyl]-ethyl}-amide

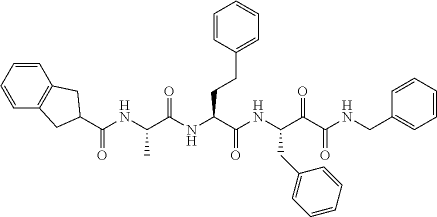 [SMILES: C[C@H](NC(=O)C1Cc2ccccc2C1)C(=O)N[C@@H](CCc1ccccc1)C(=O)N[C@@H](Cc1ccccc1)C(=O)C(=O)NCc1ccccc1]

To a solution of (S)-2-(tert-butoxycarbonylamino)-4-phenylbutanoic acid (1.5 g, 5.37 mmol) in dichloromethane (75 ml) at 0° C. were added triethylamine (1.59 ml, 11.4 mmol) and DMAP (95.1 mg, 0.78 mmol). Then a solution of benzyl chloroformate (0.92 ml, 6.44 mmol) in dichloromethane (10 ml) was added. The reaction mixture was stirred at 0° C. for 3 h and then at room temperature for 1 h. The mixture was then quenched with saturated sodium bicarbonate and extracted with dichloromethane (3×). The combined extracts were washed with water (3×) and brine and dried over magnesium sulfate. The organic phase was concentrated and the residue was purified by silica gel chromatography (10% to 20% EtOAc/hexanes) to afford 1.5 g (76%) (S)-2-tert-butoxycarbonylamino-4-phenyl-butyric acid benzyl ester as a colorless oil. LC/MS: (M+H)=370.

To a solution of (S)-2-tert-butoxycarbonylamino-4-phenyl-butyric acid benzyl ester (1.5 g, 4.0 mmol) in dichloromethane (30 ml) was added trifluoroacetic acid (9 ml). The reaction mixture was stirred at room temperature for 2.5 h and then concentrated. The residue was dissolved in DMF (20 ml) and N,N-diisopropylethylamine (5.39 ml, 30.9 mmol) was added followed by Boc-L-alanine (768 mg, 4.06 mmol) and HATU (1.7 g, 4.47 mmol). The yellow reaction mixture was stirred at room temperature for 2.5 h then quenched with water and extracted with EtOAc (3×). The combined extracts were washed with water (3×) and brine then dried over magnesium sulfate and concentrated. The residue was purified by silica gel chromatography (10% to 20% EtOAc/hexanes) to afford 0.26 g (15%) (S)-2-((S)-2-tert-butoxycarbonylamino-propionylamino)-4-phenyl-butyric acid benzyl ester. ¹H NMR (DMSO-d₆) δ: 8.26 (d, J=7.5 Hz, 1H), 7.35 (d, J=2.5 Hz, 5H), 7.21-7.29 (m, 2H), 7.13-7.20 (m, 3H), 6.85-6.99 (m, 1H), 5.10 (d, J=5.5 Hz, 2H), 4.18-4.35 (m, 1H), 3.96-4.10 (m, 1H), 2.60 (br. s., 2H), 1.87-2.02 (m, 2H), 1.38 (s, 9H), 1.16 (d, J=7.0 Hz, 3H).

To a solution of (S)-2-((S)-2-tert-butoxycarbonylamino-propionylamino)-4-phenyl-butyric acid benzyl ester (0.25 g, 0.57 mmol) in dichloromethane (30 ml) was added trifluoroacetic acid (1.09 ml). The reaction was stirred at room temperature for 2.5 h and then concentrated. The residue was dissolved in DMF (20 ml) and N,N-diisopropylethylamine (0.60 ml, 3.40 mmol) was added followed by indan-2-carboxylic acid (92 mg, 0.57 mmol) and HATU (0.237 g, 624 mmol). The brown reaction mixture was stirred at room temperature for 24 h then quenched with water. The resultant precipitate was collected via filtration, rinsed with water and dried under high vacuum to afford 270 mg (98%) (S)-2 {(S)-2-[(indane-2-carbonyl)-amino]-propionyl}-4-phenyl-butyric acid benzyl ester as off white solid. ¹H NMR (DMSO-d₆) δ: 8.39 (d, J=7.3 Hz, 1H), 8.14 (d, J=7.5 Hz, 1H), 6.95-7.46 (m, 14H), 5.11 (d, J=6.0 Hz, 2H), 4.40 (t, J=7.3 Hz, 1H), 4.18-4.30 (m, 1H), 3.27 (br. s., 1H), 3.05 (d, J=8.0 Hz, 4H), 2.58-2.64 (m, 2H), 1.85-2.08 (m, 2H), 1.21 (d, J=7.0 Hz, 3H).

To a solution of (S)-2 {(S)-2-[lindane-2-carbonyl)-amino]-propionyl}-4-phenyl-butyric acid benzyl ester (0.27 g, 0.56 mmol) in 1:1 THF/MeOH (20 ml) was added palladium hydroxide on carbon (50 mg, 0.072 mmol). The reaction was stirred under hydrogen balloon at room temperature for 2.5 h then filtered over Celite rinsing with ethyl acetate and methanol. The filtrate was concentrated to afford 130 mg (59%) (S)-2-{(S)-2-[(Indane-2-carbonyl)-amino]-propionylamino}-4-phenyl-butyric acid as an off white solid. ¹H NMR (DMSO-d₆) δ: 8.22 (t, J=8.5 Hz, 2H), 6.96-7.40 (m, 9H), 4.47 (t, J=7.2 Hz, 1H), 4.21 (dd, J=8.0, 4.3 Hz, 1H), 3.37 (br. s., 1H), 3.13 (d, J=8.0 Hz, 4H), 2.65-2.72 (m, 2H), 1.89-2.16 (m, 2H), 1.31 (dd, J=6.7, 2.4 Hz, 3H).

To a solution of ((S)-1-benzyl-2-benzylcarbamoyl-2-hydroxy-ethyl)-carbamic acid tert-butyl ester (0.142 g, 0.37 mmol) in dichloromethane (20 ml) was added trifluoroacetic acid (0.66 ml). The reaction was stirred at room temperature for 2 h and then concentrated. The residue was dissolved in DMF (20 ml) and N,N-diisopropylethylamine (0.39 ml, 2.24 mmol) was added followed by (S)-2 {(S)-2-[(indane-2-carbonyl)-amino]-propionyl}-4-phenyl-butyric acid (0.13 g, 0.33 mmol) and HATU (0.237 g, 0.62 mmol). The pale brown reaction mixture was stirred at room temperature for 24 h and quenched with water. The resultant precipitate was collected via filtration, rinsed with petroleum ether and dried under high vacuum to afford 120 mg (55%) indan-2-carboxylic acid{(S)-1-[(S)-1-((S)-benzyl-2-benzylcarbamoyl-2-hydroxy-ethylcarbamoyl)-3-phenyl-propylcarbamoyl]-ethyl}-amide as an off white solid. LC/MS: (M+H)=661.

To a solution of indan-2-carboxylic acid{(S)-1-[(S)-1-((S)-benzyl-2-benzylcarbamoyl-2-hydroxy-ethylcarbamoyl)-3-phenyl-propylcarbamoyl]-ethyl}-amide (0.12 g, 0.18 mmol) was added Dess-Martin periodinane (0.116 mg, 0.27 mmol). Within a few minutes a thick white precipitate had formed. The slurry was stirred at room temperature for 2.5 h then saturated sodium bicarbonate (4 ml) and aqueous 10% Na₂S₂O₃ (4 ml) were added. The biphasic mixture was stirred vigorously for 15 min then the layers were separated and the aqueous phase was extracted with dichloromethane (3×). The combined organics were dried over magnesium sulfate and concentrated. The crude residue was triturated with ether/dichloromethane/hexane to afford indan-2-carboxylic acid{(S)-1-[(S)-1-((S)-benzyl-2-benzylcarbamoyl-2-oxo-ethylcarbamoyl)-3-phenyl-propylcarbamoyl]-ethyl}-amide 47 mg (39%) as an off white solid. LC/MS: (M+H)=659; ¹H NMR (DMSO-d₆) δ: 9.23 (t, J=6.4 Hz, 1H), 8.34 (d, J=6.8 Hz, 1H), 8.16 (d, J=7.3 Hz, 1H), 7.94 (d, J=8.0 Hz, 1H), 7.01-7.36 (m, 19H), 5.11-5.35 (m, 1H), 4.23-4.44 (m, 4H), 3.21-3.32 (m, 2H), 3.12 (dd, J=14.1, 4.5 Hz, 1H), 3.04 (dd, J=8.3, 2.5 Hz, 4H), 2.86 (dd, J=13.9, 8.9 Hz, 1H), 1.70-1.99 (m, 2H), 1.21 (d, J=7.0 Hz, 3H).

### Example 43

### Indan-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide

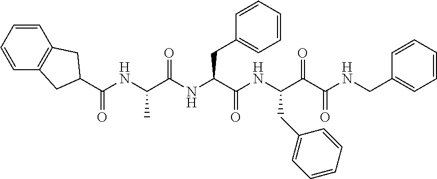 [SMILES: C[C@H](NC(=O)C1Cc2ccccc2C1)C(=O)N[C@@H](Cc1ccccc1)C(=O)N[C@@H](Cc1ccccc1)C(=O)C(=O)NCc1ccccc1]

To a solution of (S)-2-tert-butoxycarbonylamino-phenylpropionic acid (1.5 g, 5.65 mmol) in dichloromethane (75 ml) at 0° C. were added DMAP (100 mg, 0.82 mmol) and triethyl amine (1.67 ml, 12.0 mmol). Then a solution of benzyl chloroformate (0.97 ml, 6.44 mmol) in dichloromethane (10 ml) was added. The reaction mixture was stirred at 0° C. for 3 h and then room temperature for 1 h. The mixture was then quenched with saturated sodium bicarbonate and extracted with dichloromethane (3×). The combined extracts were washed with water (3×) and brine and then dried over magnesium sulfate. The organic phase was concentrated and the residue was purified by silica gel chromatography (10% to 20% EtOAc/hexanes) to afford 1.57 g (78%) (S)-2-tert-butoxycarbonylamino-3-phenyl-propionic acid benzyl ester. ¹H NMR (DMSO-d₆) δ: 7.12-7.47 (m, 10H), 5.10 (s, 2H), 4.17-4.34 (m, 1H), 2.82-3.10 (m, 2H), 1.32 (s, 9H).

To a solution of (S)-2-tert-butoxycarbonylamino-3-phenyl-propionic acid benzyl ester (1.5 g, 4.22 mmol) in dichloromethane (30 ml) was added trifluoroacetic acid (9.75 ml). The reaction mixture was stirred at room temperature for 2.5 h and then concentrated. The residue was dissolved in DMF (20 ml) and N,N-diisopropylethylamine (5.6 ml, 32.0 mmol) was added followed by Boc-L-alanine (799 mg, 4.22 mmol) and HATU (1.77 g, 4.64 mmol). The yellow reaction mixture was stirred at room temperature for 2.5 h then quenched with water and extracted with EtOAc (3×). The combined extracts were washed with water (3×) and brine and then dried over magnesium sulfate. The residue was purified by silica gel chromatography (10% to 20% EtOAc/hexanes) to afford 1.6 g (89%) (S)-2-((S)-2-tert-butoxycarbonylamino-propionylamino)-3-phenyl-propionic acid benzyl ester. ¹H NMR (DMSO-d₆) δ: 8.18 (d, J=7.5 Hz, 1H), 7.11-7.42 (m, 10H), 6.81 (d, J=7.8 Hz, 1H), 5.06 (d, J=4.5 Hz, 2H), 4.53 (q, J=7.2 Hz, 1H), 2.87-3.08 (m, 2H), 1.36 (s, 9H), 1.11 (d, J=7.0 Hz, 3H).

To a solution of (S)-2-((S)-2-tert-butoxycarbonylamino-propionylamino)-3-phenyl-propionic acid benzyl ester (0.83 g, 1.95 mmol) in dichloromethane (30 ml) was added trifluoroacetic acid (3.75 ml). The reaction was stirred at room temperature for 2.5 hours and then concentrated. The residue was dissolved in DMF (20 ml) and N,N-diisopropylethylamine (1.36 ml, 7.78 mmol) was added followed by indan-2-carboxylic acid (0.32 g, 1.97 mmol) and HATU (0.81 g, 2.14 mmol). The brown reaction mixture was stirred at room temperature for 24 h then quenched with water and extracted with EtOAc (3×). The combined extracts were washed with water (3×) and brine then dried over magnesium sulfate and concentrated. The crude residue was purified using silica gel chromatography (10% to 20% ethyl acetate/hexanes) to afford 220 mg (24%) (S)-2{(S)-2-[(indane2-carbonyl)-amino]-propionylamino}-3-phenyl-propionic acid benzyl ester as white solid. LC/MS: (M+H)=471.

To a solution of (S)-2 {(S)-2-[(indane-2-carbonyl)-amino]-propionylamino}-3-phenyl-propionic acid benzyl ester (0.22 g, 0.47 mmol) in 1:1 THF/MeOH (20 ml) was added palladium hydroxide on carbon (50 mg, 0.072 mmol). The reaction was stirred under hydrogen balloon at room temperature for 2.5 h then filtered over Celite rinsing with ethyl acetate and methanol. The filtrate was concentrated to afford 170 mg (95%) (S)-2-{(S)-2-[(Indane-2-carbonyl)-amino]-propionylamino}-3-phenyl-propionic acid as an off white solid. LC/MS: (M+H)=381.

To a solution of ((S)-1-benzyl-2-benzylcarbamoyl-2-hydroxy-ethyl)-carbamic acid tert-butyl ester (0.92 g, 0.50 mmol) in dichloromethane (20 ml) was added trifluoroacetic acid (0.9 ml). The reaction mixture was stirred at room temperature for 2 h and then concentrated. The residue was dissolved in DMF (20 ml) and N,N-diisopropylethylamine (0.53 ml, 2.24 mmol) was added followed by (S)-2-{(S)-2-[(indane-2-carbonyl)-amino]-propionylamino}-3-phenyl-propionic acid (0.17 g, 0.45 mmol) and HATU (0.187 g, 0.492 mmol). The pale brown reaction mixture was stirred at room temperature for 24 h and quenched with water. The resultant white precipitate was collected via filtration, rinsed with petroleum ether and dried under high vacuum to afford 110 mg (38%) indan-2-carboxylic acid {(S)-1-[(S)-1-((S)-1-benzyl-2-benzylcarbamoyl-2-hydroxy-ethylcarbamoyl)-2-phenyl-propylcarbamoyl]-ethyl}-amide as an off white solid. LC/MS: (M+H)=647.

To a solution of indan-2-carboxylic acid {(S)-1-[(S)-1-((S)-1-benzyl-2-benzylcarbamoyl-2-hydroxy-ethylcarbamoyl)-2-phenyl-propylcarbamoyl]-ethyl}-amide (0.110 g, 0.170 mmol) was added Dess-Martin periodinane (0.108 g, 0.272 mmol). Within a few minutes a thick white precipitate had formed. The slurry was stirred at room temperature for 2.5 h then saturated sodium bicarbonate (4 ml) and aqueous 10% Na₂S₂O₃ (4 ml) were added. The biphasic mixture was stirred vigorously for 15 min then the layers were separated and the aqueous phase was extracted with dichloromethane (3×). The combined organics were dried over magnesium sulfate and concentrated. The crude residue was triturated with ether/dichloromethane/hexane to afford 52 mg (47%) indan-2-carboxylic acid {(S)-1-[(S)-1-((S)-1-benzyl-2-benzylcarbamoyl-2-oxo-ethylcarbamoyl)-2-phenyl-propylcarbamoyl]-ethyl}-amide as an off white solid. LC/MS: (M+H)=645; ¹H NMR (DMSO-d₆) δ: 9.24 (t, J=6.1 Hz, 1H), 8.41 (d, J=6.8 Hz, 1H), 8.03 (d, J=7.3 Hz, 1H), 7.87 (d, J=8.3 Hz, 1H), 7.07-7.37 (m, 19H), 5.19-5.38 (m, 1H), 4.46-4.64 (m, 1H), 4.32 (d, J=6.0 Hz, 2H), 4.25 (t, J=7.2 Hz, 1H), 3.08-3.24 (m, 3H), 2.94-3.06 (m, 4H), 2.88 (dd, J=13.8, 8.5 Hz, 1H), 2.75 (dd, J=13.8, 9.8 Hz, 1H), 1.12 (d, J=7.0 Hz, 3H).

### Example 44

### 1-Methyl-1H-indole-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide

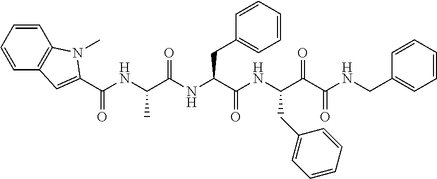 [SMILES: C[C@H](NC(=O)c1cc2ccccc2n1C)C(=O)N[C@@H](Cc1ccccc1)C(=O)N[C@@H](Cc1ccccc1)C(=O)C(=O)NCc1ccccc1]

To a solution of (S)-2-((S)-2-tert-butoxycarbonylamino-propionylamino)-3-phenyl-propionic acid benzyl ester (1.2 g, 2.81 mmol) in dichloromethane (40 ml) was added trifluoroacetic acid (4.34 ml). The reaction was stirred at room temperature for 2.0 h and then concentrated. The residue was dissolved in DMF (20 ml) and N,N-diisopropylethylamine (0.96 ml, 5.51 mmol) was added followed by 1-methyl-1H-indole-2-carboxylic acid (0.161 g, 0.919 mmol) and HATU (0.384 g, 1.01 mmol). The brown reaction mixture was stirred at room temperature for 24 h then quenched with water and extracted with EtOAc (3×). The combined extracts were dried over magnesium sulfate and concentrated. The residue was washed with water and triturated with petroleum ether to afford 330 mg (74%) (S)-2-{(S)-2-[(1-methyl-1H-indole-2-carbonyl)-amino]-propionylamino}-3-phenyl-propionic acid benzyl ester as a white solid. LC/MS: (M+H)=445.

To a solution of (S)-2-{(S)-2-[(1-methyl-1H-indole-2-carbonyl)-amino]-propionylamino}-3-phenyl-propionic acid benzyl ester (0.32 g, 0.662 mmol) in 1:1 THF/MeOH (10 ml) was added palladium hydroxide on carbon (60.4 mg, 0.09 mmol). The reaction was stirred under hydrogen balloon at room temperature for 2.5 h then filtered over Celite rinsing with ethyl acetate and methanol. The filtrate was concentrated to afford 260 mg (99%) (S)-2-{(S)-2-[(1-methyl-1H-indole-2-carbonyl)-amino]-propionylamino}-3-phenyl-propionic acid as an off white solid. LC/MS: (M+H)=394.

To a solution of ((S)-1-benzyl-2-benzylcarbamoyl-2-hydroxy-ethyl)-carbamic acid tert-butyl ester (0.188 g, 0.489 mmol) in dichloromethane (20 ml) was added trifluoroacetic acid (0.90 ml). The reaction mixture was stirred at room temperature for 2 h and then concentrated. The residue was dissolved in DMF (20 ml) and N,N-diisopropylethylamine (0.46 ml, 2.64 mmol) was added followed by (S)-2-{(S)-2-[(1-methyl-1H-indole-2-carbonyl)-amino]-propionylamino}-3-phenyl-propionic acid (0.26 g, 0.66 mmol), 1-hydroxybenzotriazole (89.3 mg, 0.66 mmol) and 1-(3-dimethylaminopropyl)-3-ethylcarbodiimide hydrochloride (0.177 g, 0.93 mmol). The reaction mixture was stirred at room temperature for 72 h and then concentrated and triturated with water and petroleum ether. The resultant precipitate was collected via filtration and dried under high vacuum to afford 300 mg (68%) 1-methyl-1H-indole-2-carboxylic acid {(S)-1-[(S)-1-((S)-1-benzyl-2-benzylcarbamoyl-2-hydroxy-ethylcarbamoyl)-2-phenyl-ethylcarbamoyl]-ethyl}-amide as a white solid. LC/MS: (M+H)=659.

To a solution of 1-methyl-1H-indole-2-carboxylic acid {(S)-1-[(S)-1-((S)-1-benzyl-2-benzylcarbamoyl-2-hydroxy-ethylcarbamoyl)-2-phenyl-ethylcarbamoyl]-ethyl}-amide (0.153 g, 0.232 mmol) was added Dess-Martin periodinane (147 mg, 0.348 mmol). Within a few minutes a thick white precipitate had formed. The slurry was stirred at room temperature for 2.5 h then saturated sodium bicarbonate (4 ml) and aqueous 10% Na₂S₂O₃ (4 ml) were added. The biphasic mixture was stirred vigorously for 15 min then the layers were separated and the aqueous phase was extracted with dichloromethane (2×). The combined organics were dried over magnesium sulfate and concentrated to afford 13 mg (8%) 1-methyl-1H-indole-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}amide as a white solid. LC/MS: (M+H)=658; ¹H NMR (DMSO-d₆) δ: 8.47 (d, J=7.5 Hz, 1H), 7.92 (d, J=8.3 Hz, 1H), 7.65 (d, J=8.0 Hz, 1H), 7.52 (d, J=8.3 Hz, 1H), 6.99-7.38 (m, 20H), 5.27 (dd, J=8.4, 4.9 Hz, 1H), 4.53-4.65 (m, 1H), 4.37-4.44 (m, 1H), 4.29-4.35 (m, 2H), 3.91 (s, 3H), 3.13 (dd, J=13.9, 4.6 Hz, 1H), 2.99 (dd, J=13.9, 4.4 Hz, 1H), 2.72-2.91 (m, 2H), 1.23 (d, J=7.0 Hz, 3H).

### Example 45

### 1-Methyl-1H-pyrrolo[3,2-1)]pyridine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide

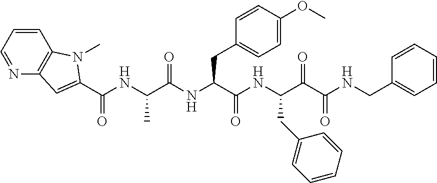 [SMILES: COc1ccc(C[C@H](NC(=O)[C@H](C)NC(=O)c2cc3ncccc3n2C)C(=O)N[C@@H](Cc2ccccc2)C(=O)C(=O)NCc2ccccc2)cc1]

In a 50 ml 2-neck round-bottomed flask, methyl 1H-pyrrolo[3,2-b]pyridine-2-carboxylate (300 mg, 1.7 mmol) was dissolved in 6.0 ml DMF. The reaction mixture was cooled to 0° C. Sodium hydride, 60% dispersion in mineral oil (85 mg, 2.13 mmol) was added at 0° C. The reaction mixture was stirred at 0° C. for 30 min. Then methyl iodide (295 mg, 0.13 ml, 2.08 mmol) was added and the reaction mixture was stirred at 0° C. for 1 h. The reaction mixture was quenched with water and extracted with 60 ml diethyl ether/EtOAc (1:1) and 5 ml water. The aqueous layer was extracted with 60 ml diethyl ether/EtOAc (1:1). The organic layers were washed twice with 5 ml water and once with 5 ml brine. The organic layers were combined, dried over sodium sulfate, filtered and concentrated. The residue was chromatographed over 25 g silica gel with EtOAc/hexanes (gradient 0-40% EtOAc). All fractions containing product were combined and concentrated to afford 206 mg (64%) 1-methyl-1H-pyrrolo[3,2-b]pyridine-2-carboxylic acid methyl ester as an off-white solid.

In a 25 ml round-bottomed flask, 1-methyl-1H-pyrrolo[3,2-b]pyridine-2-carboxylic acid methyl ester (200 mg, 1.05 mmol) was dissolved in 2.2 ml THF and 2.2 ml methanol. Lithium hydroxide (113 mg, 4.73 mmol) was added followed by 2.2 ml water. The reaction mixture was stirred at room temperature overnight. The aqueous layer was acidified with 1M HCl to pH=6 and the organic solvents were evaporated. The residue was extracted four times with 30 ml EtOAc. The organic layers were combined, dried over sodium sulfate, filtered and concentrated. The residue (˜30 mg off-white solid) was recombined with the aqueous layer, concentrated and then put under high vacuum to afford 236 mg (64%; purity=50%) 1-methyl-1H-pyrrolo[3,2-b]pyridine-2-carboxylic acid as an off-white waxy solid which was used without further purification (the isolated product contains LiCl as impurity).

In a 25 ml round-bottomed flask, (S)-2-((S)-2-tert-butoxycarbonylamino-propionylamino)-3-(4-methoxy-phenyl)-propionic acid benzyl ester (400 mg, 0.70 mmol; purity=80%) was dissolved in 6.0 ml dichloromethane. Trifluoroacetic acid (1.3 ml, 16.9 mmol) was added slowly. The reaction mixture was stirred at room temperature for 3 h. The solvent was evaporated and then put under high vacuum for 30 min. The residue (off-white solid) and 1-methyl-1H-pyrrolo[3,2-b]pyridine-2-carboxylic acid (229 mg, 0.65 mmol; purity=50%) were dissolved in 4.0 ml DMF. The light yellow solution was cooled to 0° C. N,N-Diisopropylethylamine (503 mg, 0.68 ml, 3.89 mmol) was added dropwise at 0° C. followed by HATU (272 mg, 0.72 mmol). After the addition was complete, the ice bath was removed and the yellow solution was stirred at room temperature overnight. The reaction mixture was quenched with 5 ml water and extracted twice with 60 ml diethyl ether/EtOAc (1:1). The organic layers were washed twice with 5 ml water and once with 5 ml brine. The organic layers were combined, dried over sodium sulfate, filtered and concentrated. The residue was chromatographed over 25 g silica gel with EtOAc/hexanes (gradient 0-100% EtOAc). All fractions containing product were combined and concentrated to afford 264 mg (79%) (S)-3-(4-methoxy-phenyl)-2-{(S)-2-[(1-methyl-1H-pyrrolo[3,2-b]pyridine-2-carbonyl)-amino]-propionylamino}-propionic acid benzyl ester as an off-white solid.

In a 50 ml round-bottomed flask, (S)-3-(4-methoxy-phenyl)-2-{(S)-2-[(1-methyl-1H-pyrrolo[3,2-b]pyridine-2-carbonyl)-amino]-propionylamino}-propionic acid benzyl ester (262 mg, 0.51 mmol) was dissolved in 5.2 ml methanol and 2.6 ml THF. The flask was three times alternating evacuated and flushed with argon. 20% Palladium hydroxide on carbon (wet, 47 mg, 0.07 mmol) was added carefully. The flask was evacuated, flushed with argon, evacuated and flushed with hydrogen. The reaction mixture was stirred under hydrogen atmosphere (balloon) at room temperature for 2 h (the product precipitated). The reaction mixture was diluted with methanol and ethyl acetate and heated to dissolve the desired product. The suspension was filtered and rinsed with warm methanol/ethyl acetate. The filtrate was concentrated to afford 199 mg (92%) (S)-3-(4-methoxy-phenyl)-2-{(S)-2-[(1-methyl-1H-pyrrolo[3,2-b]pyridine-2-carbonyl)-amino]-propionylamino}-propionic acid as an off-white solid.

In a 10 ml round-bottomed flask, ((S)-1-benzyl-2-benzylcarbamoyl-2-hydroxy-ethyl)-carbamic acid tert-butyl ester (125 mg, 0.33 mmol) was dissolved in 3.0 ml dichloromethane. Trifluoroacetic acid (0.60 ml, 7.79 mmol) was added slowly. The reaction mixture was stirred at room temperature for 3 h. The solvent was evaporated and then placed under high vacuum for 1 h. The residue and (S)-3-(4-methoxy-phenyl)-2-{(S)-2-[(1-methyl-1H-pyrrolo[3,2-b]pyridine-2-carbonyl)-amino]-propionylamino}-propionic acid (125 mg, 0.29 mmol) were dissolved in 2.0 ml DMF and cooled to 0° C. N,N-Diisopropylethylamine (222 mg, 0.30 ml, 1.72 mmol) was added dropwise at 0° followed by HATU (112 mg, 0.29 mmol). After the addition was complete, the ice bath was removed and the reaction mixture was stirred at room temperature overnight. The reaction mixture was diluted with water (a precipitate was formed). The suspension was filtered, rinsing with water and a little petroleum ether. The resulting light brown solid was dried using the rotavap and then placed under high vacuum to afford 144 mg (71%) 1-methyl-1H-pyrrolo[3,2-b]pyridine-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide as a mixture of epimers.

In a 50 ml round-bottomed flask, 1-methyl-1H-pyrrolo[3,2-b]pyridine-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide (136 mg, 0.20 mmol) was partially dissolved in 8.0 ml dichloromethane and Dess-Martin periodinane (125 mg, 0.30 mmol) was added. The reaction mixture was stirred at room temperature for 2 h. The reaction mixture was quenched with 3.5 ml saturated NaHCO₃-solution and 3.5 ml 10% Na₂S₂O₃-solution and stirred vigorously for 30 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was triturated with dichloromethane/diethyl ether/ethyl acetate to afford 74 mg (49%; purity=90%) 1-methyl-1H-pyrrolo[3,2-b]pyridine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide as a yellow solid. LC/MS: (M−H)⁻=687.

### Example 46

### (S)—N-Benzyl-3-{(S)-3-(4-methoxy-phenyl)-2-[(S)-2-(2-morpholin-4-yl-acetylamino)-propionylamino]-propionylamino}-2-oxo-4-phenyl-butyramide

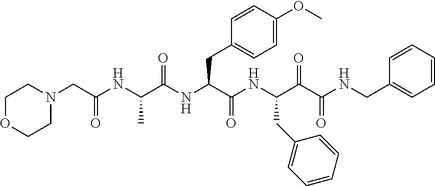 [SMILES: COc1ccc(C[C@H](NC(=O)[C@H](C)NC(=O)CN2CCOCC2)C(=O)N[C@@H](Cc2ccccc2)C(=O)C(=O)NCc2ccccc2)cc1]

A solution of (S)-3-amino-N-benzyl-2-hydroxy-4-phenyl-butyramide trifluoroacetate (100 mg, 0.25 mmol) in DMF (2.5 ml) was cooled to 0° C. and stirred for 5 min. N,N-Diisopropylethylamine (0.24 ml, 1.37 mmol) was added dropwise followed by (S)-3-(4-methoxy-phenyl)-2-[(S)-2-(2-morpholin-4-yl-acetylamino)-propionylamino]-propionic acid (90 mg, 0.23 mmol) and HATU (96 mg, 0.25 mmol). After the addition was complete, the ice-bath removed and the reaction mixture was stirred at room temperature for 18 h then diluted with water and extracted with EtOAc. The organics were washed with brine, dried over MgSO₄ and concentrated. The residue was purified by chromatography (silica gel, gradient: 0-10% MeOH/dichloromethane) to obtain 140 mg (93%) of (S)—N-benzyl-2-hydroxy-3-{(S)-3-(4-methoxy-phenyl)-2-[(S)-2-(2-morpholin-4-yl-acetylamino)-propionylamino]-propionylamino}-4-phenyl-butyramide as a light brown powder.

To a solution of (S)—N-benzyl-2-hydroxy-3-{(S)-3-(4-methoxy-phenyl)-2-[(S)-2-(2-morpholin-4-yl-acetylamino)-propionylamino]-propionylamino}-4-phenyl-butyramide (140 mg, 0.21 mmol) in dichloromethane (10 ml) was added Dess-Martin periodinane (99 mg, 0.23 mmol). The reaction mixture was stirred at room temperature for 4 h then quenched with 4 ml of satd. NaHCO₃ solution and 4 ml of 10% Na₂S₂O₃ solution. The biphasic mixture was stirred vigorously for 30 min then extracted with dichloromethane. The organics were washed with satd. NaHCO₃ solution, dried (Na₂SO₄) and concentrated. The residue was purified by chromatography (silica gel, gradient: 0-10% MeOH/dichloromethane) to obtain 43 mg (27%) of (S)—N-benzyl-3-{(S)-3-(4-methoxy-phenyl)-2-[(S)-2-(2-morpholin-4-yl-acetylamino)-propionylamino]-propionylamino}-2-oxo-4-phenyl-butyramide as a light yellow powder. LC/MS: (M+H)⁺=658.

### Example 47

### 5-Methyl-isoxazole-3-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide

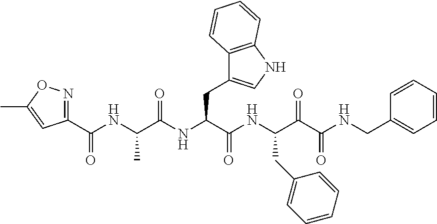 [SMILES: Cc1cc(C(=O)N[C@@H](C)C(=O)N[C@@H](Cc2c[nH]c3ccccc23)C(=O)N[C@@H](Cc2ccccc2)C(=O)C(=O)NCc2ccccc2)no1]

A solution of (S)-3-amino-N-benzyl-2-hydroxy-4-phenyl-butyramide trifluoroacetate (131 mg, 0.33 mmol) in DMF (2 ml) was cooled to 0° C. and stirred for 5 min. N,N-Diisopropylethylamine (0.31 ml, 1.8 mmol) was added dropwise followed by (S)-3-(1H-indol-3-yl)-2-{(S)-2-[(5-methyl-isoxazole-3-carbonyl)-amino]-propionylamino}-propionic acid (115 mg, 0.30 mmol) and HATU (125 mg, 0.33 mmol). After the addition was complete, the ice-bath was removed and the reaction mixture was stirred at room temperature for 18 h then diluted with water and extracted with EtOAc. The organics were washed with brine, dried (MgSO₄) and concentrated. The residue was purified by chromatography (silica gel, gradient: 0-10% MeOH/dichloromethane) to obtain 163 mg (83%) of 5-methyl-isoxazole-3-carboxylic acid {(S)-1-[(S)-1-((S)-1-benzyl-2-benzylcarbamoyl-2-hydroxy-ethylcarbamoyl)-2-(1H-indol-3-yl)-ethylcarbamoyl]-ethyl}-amide as a light yellow powder.

To a solution of 5-methyl-isoxazole-3-carboxylic acid {(S)-1-[(S)-1-((S)-1-benzyl-2-benzylcarbamoyl-2-hydroxy-ethylcarbamoyl)-2-(1H-indol-3-yl)-ethylcarbamoyl]-ethyl}-amide (160 mg, 0.25 mmol) in dichloromethane (8 ml) at 0° C. was added Dess-Martin periodinane (115 mg, 0.27 mmol). The reaction mixture was stirred at room temperature for 4 h then quenched with ˜4 ml satd. NaHCO₃ solution and ˜4 ml of 10% Na₂S₂O₃ solution. The biphasic mixture was stirred vigorously for 30 min then extracted with dichloromethane. The organics were washed with satd. NaHCO₃ solution, dried (Na₂SO₄) and concentrated. The residue was purified by chromatography (silica gel, gradient: 0-10% MeOH/dichloromethane) to obtain 15 mg (9%) of 5-methyl-isoxazole-3-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide as a light yellow powder. LC/MS: (M+H)⁺=649

### Example 48

### 3-Methyl-1H-indene-2-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide

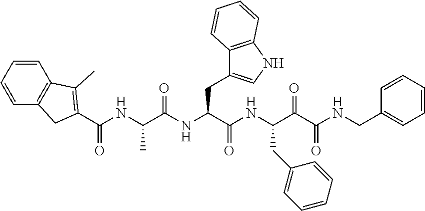 [SMILES: CC1=C(C(=O)N[C@@H](C)C(=O)N[C@@H](Cc2c[nH]c3ccccc23)C(=O)N[C@@H](Cc2ccccc2)C(=O)C(=O)NCc2ccccc2)Cc2ccccc21]

A microwave vial was charged with (S)-3-(1H-indol-3-yl)-2-{(S)-2-[(3-methyl-1H-indene-2-carbonyl)-amino]-propionylamino}-propionic acid tert-butyl ester (290 mg, 0.60 mmol) and 1,1,1,3,3,3-hexafluoro-2-propanol (2.75 ml, 26.1 mmol). The vial was flushed with nitrogen and sealed. The colorless solution was heated at 120° C. for 4 h under microwave irradiation.

The reaction mixture was concentrated to afford (S)-3-(1H-indol-3-yl)-2-{(S)-2-[(3-methyl-1H-indene-2-carbonyl)-amino]-propionylamino}-propionic acid as an off-white foam and used without further purification

A solution of (S)-3-amino-N-benzyl-2-hydroxy-4-phenyl-butyramide trifluoroacetate (147 mg, 0.37 mmol) in DMF (2 ml) was cooled to 0° C. and stirred for 5 min. N,N-Diisopropylethylamine (0.35 ml, 2.0 mmol) was added dropwise followed by a solution of (S)-3-(1H-indol-3-yl)-2-{(S)-2-[(3-methyl-1H-indene-2-carbonyl)-amino]-propionylamino}-propionic acid (145 mg, 0.34 mmol) in DMF (1 ml) and HATU (141 mg, 0.37 mmol). After the addition was complete, the ice-bath was removed and the reaction mixture was stirred at room temperature for 18 h then diluted with water and extracted with EtOAc. The organics were washed with brine, dried (MgSO₄) and concentrated. The residue was purified by chromatography (silica gel, gradient: 0-10% MeOH/dichloromethane) to obtain 178 mg (76%) of 3-methyl-1H-indene-2-carboxylic acid {(S)-1-[(S)-1-((S)-1-benzyl-2-benzylcarbamoyl-2-hydroxy-ethylcarbamoyl)-2-(1H-indol-3-yl)-ethylcarbamoyl]-ethyl}-amide as an off-white powder.

To a solution of 3-methyl-1H-indene-2-carboxylic acid {(S)-1-[(S)-1-((S)-1-benzyl-2-benzylcarbamoyl-2-hydroxy-ethylcarbamoyl)-2-(1H-indol-3-yl)-ethylcarbamoyl]-ethyl}-amide (73 mg, 0.105 mmol) in dichloromethane (10 ml) at 0° C. was added Dess-Martin periodinane (49 mg, 0.115 mmol). The reaction mixture was stirred at 0° C. for 2 h then at room temperature for 4 h. The reaction was quenched with satd. NaHCO₃ solution and 10% Na₂S₂O₃ solution. The biphasic mixture was stirred vigorously for 30 min then extracted with dichloromethane. The organics were washed with satd. NaHCO₃ solution, dried (Na₂SO₄) and concentrated. The residue was purified by chromatography (silica gel, gradient: 1-5% MeOH/dichloromethane) to obtain 7 mg (9%) of 3-methyl-1H-indene-2-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide as an off-white powder. LC/MS: (M+H)⁺=696.

### Example 49

### Imidazo[1,2-a]pyridine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide

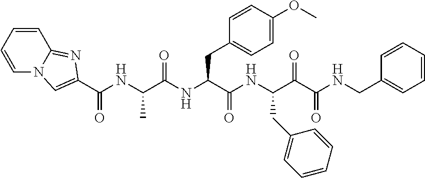 [SMILES: COc1ccc(C[C@H](NC(=O)[C@H](C)NC(=O)c2cn3ccccc3n2)C(=O)N[C@@H](Cc2ccccc2)C(=O)C(=O)NCc2ccccc2)cc1]

To a solution of (S)-2-tert-butoxycarbonylamino-3-(4-methoxy-phenyl)-propionic acid methyl ester (1.01 g, 3.26 mmol) in dichloromethane (10 ml) was added TFA (5.03 ml, 65.3 mmol). The reaction mixture was stirred at room temperature for 2.5 h then the solvent was removed in vacuo. The residue was treated with a 50% Et₂O-petroleum ether mixture and the resultant precipitate was filtered and dried to obtain 1.01 g (96%) of (S)-2-amino-3-(4-methoxy-phenyl)-propionic acid methyl ester trifluoroacetate as a white powder.

To a solution of (S)-2-amino-3-(4-methoxy-phenyl)-propionic acid methyl ester trifluoroacetate (1.01 g, 3.12 mmol) in DMF (10 ml) at 0° C. was added N,N-diisopropylethylamine (5.46 ml, 31.2 mmol) dropwise. The reaction mixture was stirred for 5 min then (S)-2-(tert-butoxycarbonylamino)propanoic acid (591 mg, 3.12 mmol) was added followed by HATU (1.31 g, 3.44 mmol). After the addition was complete, the ice-bath removed and the reaction mixture was stirred at room temperature for 18 h then quenched with water and extracted with EtOAc. The organics were washed with brine, dried (MgSO₄) and concentrated. The residue was purified by chromatography (silica gel, gradient: 10-50% EtOAc/hexanes) to obtain 1.07 g (90%) of (S)-2-((S)-2-tert-butoxycarbonylamino-propionylamino)-3-(4-methoxy-phenyl)-propionic acid methyl ester as light yellow foam.

To a solution of (S)-2-((S)-2-tert-butoxycarbonylamino-propionylamino)-3-(4-methoxy-phenyl)-propionic acid methyl ester (0.65 g, 1.71 mmol) in dichloromethane (10 ml) was added TFA (3.3 ml, 42.7 mmol). The reaction mixture was stirred at room temperature for 2.5 h then the solvent was removed in vacuo. The residue was treated with a 50% Et₂O-petroleum ether mixture and the resultant precipitate was filtered, washed with ether and dried to obtain 0.66 g (98%) of (S)-2-((S)-2-amino-propionylamino)-3-(4-methoxy-phenyl)-propionic acid methyl ester trifluoroacetate as an off-white powder.

A solution of (S)-2-((S)-2-amino-propionylamino)-3-(4-methoxy-phenyl)-propionic acid methyl ester trifluoroacetate (250 mg, 0.63 mmol) in DMF (4 ml) was cooled to 0° C. and stirred for 5 min. N,N-Diisopropylethylamine was added dropwise at 0° C. followed by a solution of imidazo[1,2-a]pyridine-2-carboxylic acid (113 mg, 0.70 mmol) in DMF (1 ml) and HATU (265 mg, 0.70 mmol). After the addition, the ice-bath was removed and the reaction mixture was stirred at room temperature for 18 h then quenched with water and extracted with EtOAc. The organics were washed with brine, dried (MgSO₄) and concentrated. The residue was purified by chromatography (silica gel, gradient: 2-10% MeOH/dichloromethane) to obtain 177 mg (66%) of (S)-2-{(S)-2-[(imidazo[1,2-a]pyridine-2-carbonyl)-amino]-propionylamino}-3-(4-methoxy-phenyl)-propionic acid methyl ester as an off-white foam.

To a solution of (S)-2-{(S)-2-[(imidazo[1,2-a]pyridine-2-carbonyl)-amino]-propionylamino}-3-(4-methoxy-phenyl)-propionic acid methyl ester (177 mg, 0.42 mmol) in 1,2-dichloroethane (10 ml) was added trimethyltin hydroxide (302 mg, 1.67 mmol). The reaction mixture was heated at 80° C. for 4 h then concentrated under reduced pressure. The residue was dissolved in dichloromethane and washed with 1M HCl then dried over MgSO₄ and concentrated to obtain 160 mg (94%) of (S)-2-{(S)-2-[(imidazo[1,2-a]pyridine-2-carbonyl)-amino]-propionylamino}-3-(4-methoxy-phenyl)-propionic acid as an off-white powder.

To a mixture of (S)-3-amino-N-benzyl-2-hydroxy-4-phenyl-butyramide trifluoroacetate (161 mg, 0.40 mmol) in DMF (2.5 ml) at 0° C. was added N,N-diisopropylethylamine (0.67 ml, 3.85 mmol) dropwise. Then (S)-2-{(S)-2-[(imidazo[1,2-a]pyridine-2-carbonyl)-amino]-propionylamino}-3-(4-methoxy-phenyl)-propionic acid (158 mg, 0.39 mmol) was added, followed by HATU (161 mg, 0.42 mmol). After the addition was completed, the ice-bath was removed and the reaction mixture was stirred at room temperature for 18 h then quenched by the addition of ice. The resultant solid was collected by filtration, washed with cold water and Et₂O, and dried. The crude solid was purified by chromatography (silica gel, gradient: 1-15% MeOH/dichloromethane) to obtain 131 mg (50%) of imidazo[1,2-a]pyridine-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide as an off-white powder.

To a solution of imidazo[1,2-a]pyridine-2-carboxylic acid {(S)-1-[(S)-1-((S)-1-benzyl-2-benzylcarbamoyl-2-hydroxy-ethylcarbamoyl)-2-(4-methoxy-phenyl)-ethylcarbamoyl]-ethyl}-amide (131 mg, 0.19 mmol) in dichloromethane (10 ml) was added Dess-Martin periodinane (123 mg, 0.29 mmol). The cloudy mixture was stirred at room temperature for 4 h then quenched with 10% aqueous Na₂S₂O₃ and satd. NaHCO₃. The biphasic mixture was stirred vigorously for 30 min, then diluted with water and extracted with dichloromethane. The organics were washed with NaHCO₃, dried over MgSO₄ and concentrated. The residue was triturated with Et₂O to obtain 96 mg (74%) of imidazo[1,2-a]pyridine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide as a light yellow powder. LC/MS: (M+H)⁺=675.

### Example 50

### {(S)-1-[(S)-1-((S)-1-Benzyl-2-benzylcarbamoyl-2-oxo-ethylcarbamoyl)-2-pyridin-2-yl-ethylcarbamoyl]-ethyl}-carbamic acid benzyl ester

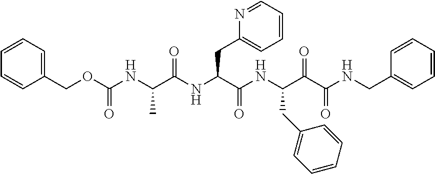 [SMILES: C[C@H](NC(=O)OCc1ccccc1)C(=O)N[C@@H](Cc1ccccn1)C(=O)N[C@@H](Cc1ccccc1)C(=O)C(=O)NCc1ccccc1]

To a solution of (S)-2-(tert-butoxycarbonylamino)-3-(pyridin-2-yl)propanoic acid (705 mg, 2.65 mmol) in MeOH (15 ml) was added dropwise trimethylsilyldiazomethane (2.0 M in hexane, 7.9 ml, 15.9 mmol). The reaction mixture was stirred for 30 min at room temperature at which time a pale yellow color persisted. The reaction was quenched with a few drops of acetic acid where upon solution became colorless. The mixture was concentrated to obtain 740 mg of (S)-2-tert-butoxycarbonylamino-3-pyridin-2-yl-propionic acid methyl ester as a brown oil which was used without further purification.

To a solution of (S)-2-tert-butoxycarbonylamino-3-pyridin-2-yl-propionic acid methyl ester (740 mg, 2.65 mmol) in dichloromethane (15 ml) was added TFA (3.06 ml, 39.7 mmol). The reaction mixture was stirred at room temperature for 2 h then concentrated, chased with hexanes and dried under high vacuum. The residue was dissolved in DMF (5 ml) and cooled to 0° C. N,N-Diisopropylethylamine (4.6 ml, 26.5 mmol) was added and the mixture was stirred for 5 min. Then added (S)-2-(benzyloxycarbonylamino)propanoic acid (591 mg, 2.65 mmol) followed by HATU (1.11 g, 2.91 mmol). The reaction mixture was warmed to room temperature and stirred overnight then quenched with cold water. The resultant precipitate was collected via filtration and dried to obtain 1.01 g (99%) of (S)-2-((S)-2-benzyloxycarbonylamino-propionylamino)-3-pyridin-2-yl-propionic acid methyl ester as an off-white powder.

To a solution of (S)-2-((S)-2-benzyloxycarbonylamino-propionylamino)-3-pyridin-2-yl-propionic acid methyl ester (1.01 g, 2.62 mmol) in 1,2-dichloroethane (20 ml) was added trimethyltin hydroxide (1.9 g, 10.5 mmol). The cloudy mixture was heated at 85° C. for 4 h then the solvent was removed in vacuo. The residue was dissolved in EtOAc and washed with 1M HCl then dried over MgSO₄ and concentrated to give 630 mg (65%) of (S)-2-((S)-2-benzyloxycarbonylamino-propionylamino)-3-pyridin-2-yl-propionic acid as a light brown oil.

To a solution of ((S)-1-benzyl-2-benzylcarbamoyl-2-hydroxy-ethyl)-carbamic acid tert-butyl ester (214 mg, 0.56 mmol) in dichloromethane (10 ml) was added TFA (0.975 ml, 12.7 mmol). The reaction mixture was stirred at room temperature for 2 h then the solvent was removed in vacuo. The residue was dried under high vacuum then dissolved in DMF (5 ml). To this solution were added (S)-2-((S)-2-benzyloxycarbonylamino-propionylamino)-3-pyridin-2-yl-propionic acid (188 mg, 0.51 mmol), HATU (212 mg, 0.56 mmol) and N,N-diisopropylethylamine (0.88 ml, 5.06 mmol). The reaction mixture was stirred at room temperature for 4 h then quenched with water and extracted with EtOAc. The organic layer was dried over MgSO₄ and concentrated. The residue was purified by chromatography (silica gel, gradient: 0-5% MeOH/dichloromethane) to obtain 198 mg (61%) of {(S)-1-[(S)-1-((S)-1-benzyl-2-benzylcarbamoyl-2-hydroxy-ethylcarbamoyl)-2-pyridin-2-yl-ethylcarbamoyl]-ethyl}-carbamic acid benzyl ester as a light yellow oil.

To a solution of {(S)-1-[(S)-1-((S)-1-benzyl-2-benzylcarbamoyl-2-hydroxy-ethylcarbamoyl)-2-pyridin-2-yl-ethylcarbamoyl]-ethyl}-carbamic acid benzyl ester (198 mg, 0.31 mmol) in dichloromethane (10 ml) was added Dess-Martin periodinane (145 mg, 0.34 mmol). The cloudy mixture was stirred at room temperature for 4 h then quenched with 10% aqueous Na₂S₂O₃ and satd. NaHCO₃. The biphasic mixture was stirred vigorously for 30 min, then diluted with water and extracted with dichloromethane. The organics were washed with satd. NaHCO₃, dried over MgSO₄ and concentrated. The residue was purified by chromatography (silica gel, gradient: 0% to 5% MeOH/dichloromethane) to obtain 27 mg (14%) of {(S)-1-[(S)-1-((S)-1-benzyl-2-benzylcarbamoyl-2-oxo-ethylcarbamoyl)-2-pyridin-2-yl-ethylcarbamoyl]-ethyl}-carbamic acid benzyl ester as a light yellow powder. LC/MS: (M+H)⁺=636.

### Example 51

### {(S)-1-[(S)-1-((S)-1-Benzyl-2-benzylcarbamoyl-2-oxo-ethylcarbamoyl)-2-(4-chloro-phenyl)-ethylcarbamoyl]-ethyl}-carbamic acid benzyl ester

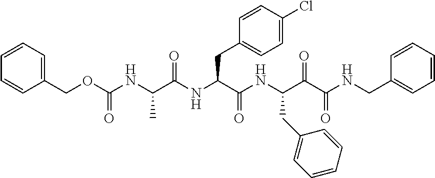 [SMILES: C[C@H](NC(=O)OCc1ccccc1)C(=O)N[C@@H](Cc1ccc(Cl)cc1)C(=O)N[C@@H](Cc1ccccc1)C(=O)C(=O)NCc1ccccc1]

To a solution of (S)-2-(tert-butoxycarbonylamino)-3-(4-chlorophenyl)propanoic acid (2.5 g, 8.34 mmol) in MeOH (40 ml) was added dropwise trimethylsilyldiazomethane (2.0M in hexane, 25.0 ml, 50.0 mmol). The reaction mixture was stirred for 30 min at room temperature at which time a pale yellow color persisted. The reaction was quenched with a few drops of acetic acid where upon the solution became colorless. The mixture was concentrated to obtain 2.62 g of (S)-2-tert-butoxycarbonylamino-3-(4-chloro-phenyl)-propionic acid methyl ester as a light yellow solid which was used without further purification.

To a solution of (S)-methyl 2-(tert-butoxycarbonylamino)-3-(4-chlorophenyl)propanoate (2.62 g, 8.35 mmol) in dichloromethane (50 ml) was added TFA (9.6 ml, 125 mmol). The reaction mixture was stirred at room temperature for 2 h then concentrated, chased with hexanes and dried under high vacuum. The residue was dissolved DMF (20 ml) and cooled to 0° C. N,N-Diisopropylethylamine (14.6 ml, 83.3 mmol) was added and the mixture was stirred for 5 min. Then added (S)-2-(benzyloxycarbonylamino)propanoic acid (1.86 g, 8.33 mmol) followed by HATU (3.49 g, 9.17 mmol). The reaction mixture was warmed to room temperature and stirred overnight then quenched with water and extracted with EtOAc. The organics were dried over MgSO₄ and concentrated. The residue was purified by chromatography (silica gel, gradient: 10-75% EtOAc/hexanes) to obtain 1.78 g (51%) of (S)-2-((S)-2-benzyloxycarbonylamino-propionylamino)-3-(4-chloro-phenyl)-propionic acid methyl ester as a white powder.

To a solution of (S)-2-((S)-2-benzyloxycarbonylamino-propionylamino)-3-(4-chloro-phenyl)-propionic acid methyl ester (550 mg, 1.31 mmol) in 1,2-dichloroethane (10 ml) was added trimethyltin hydroxide (950 mg, 5.25 mmol). The cloudy mixture was heated at 85° C. for 4 h then the solvent was removed in vacuo. The residue was dissolved in EtOAc and washed with 1M HCl then dried over MgSO₄ and concentrated to obtain 395 mg (74%) of (S)-2-((S)-2-benzyloxycarbonylamino-propionylamino)-3-(4-chloro-phenyl)-propionic acid as a white powder.

To a solution of (S)-3-amino-N-benzyl-2-hydroxy-4-phenyl-butyramide trifluoroacetate (102 mg, 0.26 mmol) in DMF (2 ml) at 0° C. was added N,N-diisopropylethylamine (0.22 ml, 1.28 mmol). The reaction was stirred for 5 min then (S)-2-((S)-2-benzyloxycarbonylamino-propionylamino)-3-(4-chloro-phenyl)-propionic acid (104 mg, 0.26 mmol) and HATU (107 mg, 0.28 mmol) were added. The ice-bath was removed and the reaction mixture was stirred at room temperature for 4 h then quenched with water. The resultant precipitate was filtered, washed with water and dried. The crude solid was triturated with Et₂O to obtain 128 mg (74%) of {(S)-1-[(S)-1-((S)-1-benzyl-2-benzylcarbamoyl-2-hydroxy-ethylcarbamoyl)-2-(4-chloro-phenyl)-ethylcarbamoyl]-ethyl}-carbamic acid benzyl ester as an off-white powder.

To a solution of {(S)-1-[(S)-1-((S)-1-benzyl-2-benzylcarbamoyl-2-hydroxy-ethylcarbamoyl)-2-(4-chloro-phenyl)-ethylcarbamoyl]-ethyl}-carbamic acid benzyl ester (128 mg, 0.19 mmol) in dichloromethane (5 ml) was added Dess-Martin periodinane (89 mg, 0.21 mmol). The cloudy reaction mixture was stirred at room temperature for 6 h then quenched with 10% aq. Na₂S₂O₃ and saturated NaHCO₃. The biphasic mixture was stirred vigorously for 30 min, then diluted with water and extracted with dichloromethane. The organic phase was washed with saturated NaHCO₃, then dried over MgSO₄ and concentrated. The residue was triturated with Et₂O to obtain 82 mg (64%) of {(S)-1-[(S)-1-((S)-1-benzyl-2-benzylcarbamoyl-2-oxo-ethylcarbamoyl)-2-(4-chloro-phenyl)-ethylcarbamoyl]-ethyl}-carbamic acid benzyl ester as a white powder. LC/MS: (M+H)⁺=670.

### Example 52

### 1,3-Dihydro-isoindole-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide

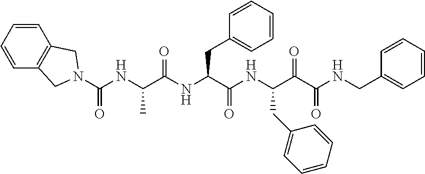 [SMILES: C[C@H](NC(=O)N1Cc2ccccc2C1)C(=O)N[C@@H](Cc1ccccc1)C(=O)N[C@@H](Cc1ccccc1)C(=O)C(=O)NCc1ccccc1]

To a solution of triphosgene (2.49 g, 8.39 mmol) in dichloromethane (10 ml) at 0° C. was added pyridine (0.68 ml, 8.39 mmol). Then a solution of isoindoline (1.0 g, 8.39 mmol) in 5 ml of dichloromethane was added over a period of 10 min. The reaction mixture was allowed to warm to room temperature and stirred for 2 h then quenched by careful addition of 1N HCl (10 ml). The organic phase was separated and washed with satd. NaHCO₃ solution, then dried over Na₂SO₄ and concentrated. The residue was purified by chromatography (silica gel, gradient: 0-25% EtOAc/hexanes) to obtain 420 mg (28%) of 1,3-dihydro-isoindole-2-carbonyl chloride as an off-white powder.

To a mixture of (S)-2-((S)-2-tert-butoxycarbonylamino-propionylamino)-3-phenyl-propionic acid benzyl ester (0.61 g, 1.43 mmol) and palladium hydroxide on carbon (26 mg, 0.19 mmol) was carefully added MeOH (5 ml). The reaction mixture stirred under hydrogen, using a balloon (1 atm) for 2 h. The reaction was filtered through a bed of Celite, washed with MeOH and concentrated to obtain 480 mg of (S)-2-((S)-2-tert-butoxycarbonylamino-propionylamino)-3-phenyl-propionic acid as a white foam.

To a solution of ((S)-1-benzyl-2-benzylcarbamoyl-2-hydroxy-ethyl)-carbamic acid tert-butyl ester (454 mg, 1.18 mmol) in dichloromethane (10 ml) was added TFA (1.8 ml, 23.6 mmol). The reaction mixture was stirred at room temperature for 2 h then concentrated and dried under high vacuum. The residue was dissolved in DMF (5 ml) and (S)-2-((S)-2-tert-butoxycarbonylamino-propionylamino)-3-phenyl-propionic acid (437 mg, 1.3 mmol), HATU (494 mg, 1.3 mmol) and N,N-diisopropylethylamine (1.03 ml, 5.9 mmol) were added. The reaction mixture was stirred at room temperature for 2 h then quenched with water. The resultant precipitate was filtered and washed with water then dried under high vacuum. The crude solid was triturated with Et₂O to obtain 602 mg (85%) of {(S)-1-[(S)-1-((S)-1-benzyl-2-benzylcarbamoyl-2-hydroxy-ethylcarbamoyl)-2-phenyl-ethylcarbamoyl]-ethyl}-carbamic acid tert-butyl ester as an off-white powder.

To a solution of {(S)-1-[(S)-1-((S)-1-benzyl-2-benzylcarbamoyl-2-hydroxy-ethylcarbamoyl)-2-phenyl-ethylcarbamoyl]-ethyl}-carbamic acid tert-butyl ester (601 mg, 1.0 mmol) in dichloromethane (25 ml) at room temperature was added Dess-Martin periodinane (465 mg, 1.1 mmol). The cloudy mixture was stirred at room temperature for 3 h then quenched with 10% aq. Na₂S₂O₃ and satd. NaHCO₃. The biphasic mixture was stirred vigorously for 30 min, then diluted with water and extracted with dichloromethane. The organics were washed with satd. NaHCO₃ and dried over MgSO₄ then concentrated. The residue was purified by chromatography (silica gel, gradient: 0% to 5% MeOH/dichloromethane) to obtain 369 mg (62%) of {(S)-1-[(S)-1-((S)-1-benzyl-2-benzylcarbamoyl-2-oxo-ethylcarbamoyl)-2-phenyl-ethylcarbamoyl]-ethyl}-carbamic acid tert-butyl ester as an off-white powder.

To a solution of {(S)-1-[(S)-1-((S)-1-benzyl-2-benzylcarbamoyl-2-oxo-ethylcarbamoyl)-2-phenyl-ethylcarbamoyl]-ethyl}-carbamic acid tert-butyl ester (114 mg, 0.19 mmol) in dichloromethane (5 ml) was added TFA (0.29 ml, 3.8 mmol). The reaction mixture was stirred at room temperature for 2 h then concentrated and dried under high vacuum. The residue was dissolved in dichloromethane (5 ml) and N,N-diisopropylethylamine (0.73 ml, 4.18 mmol) was added, followed by 1,3-dihydro-isoindole-2-carbonyl chloride (35 mg, 0.19 mmol). The reaction mixture was stirred at room temperature for 2 h then quenched with water and extracted with dichloromethane. The organic phase was dried over MgSO₄ and concentrated. The residue was purified by chromatography (silica gel, gradient: 0-5% MeOH/dichloromethane) to obtain 27 mg (22%) of 1,3-dihydro-isoindole-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide as a white powder. LC/MS: (M+H)⁺=646.

### Example 53

### 2,5-Dimethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide

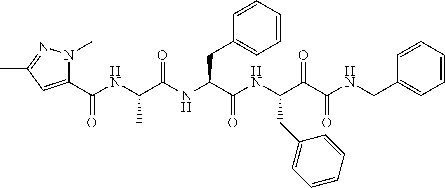 [SMILES: Cc1cc(C(=O)N[C@@H](C)C(=O)N[C@@H](Cc2ccccc2)C(=O)N[C@@H](Cc2ccccc2)C(=O)C(=O)NCc2ccccc2)n(C)n1]

To a solution of {(S)-1-[(S)-1-((S)-1-benzyl-2-benzylcarbamoyl-2-oxo-ethylcarbamoyl)-2-phenyl-ethylcarbamoyl]-ethyl}-carbamic acid tert-butyl ester (252 mg, 0.42 mmol) in dichloromethane (10 ml) was added TFA (0.65 ml, 8.39 mmol). The reaction mixture was stirred at room temperature for 2 h then concentrated and dried under high vacuum. The residue was triturated with Et₂O to afford 247 mg (95%) of (S)-3-[(S)-2-((S)-2-amino-propionylamino)-3-phenyl-propionylamino]-N-benzyl-2-oxo-4-phenyl-butyramide trifluoroacetate as an off-white powder.

To a solution of (S)-3-[(S)-2-((S)-2-amino-propionylamino)-3-phenyl-propionylamino]-N-benzyl-2-oxo-4-phenyl-butyramide trifluoroacetate (100 mg, 0.16 mmol) in DMF (2 ml) was added 1,3-dimethyl-1H-pyrazole-5-carboxylic acid (24 mg, 0.17 mmol), HATU (68 mg, 0.18 mmol) and N,N-diisopropylethylamine (0.28 ml, 1.63 mmol). The reaction mixture was stirred at room temperature for 1 h then quenched with cold water. The resultant precipitate was filtered, washed with water and Et₂O and dried to obtain 76 mg (75%) of 2,5-dimethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide as white powder. LC/MS: (M+H)⁺=623.

### Example 54

### 2-Ethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide

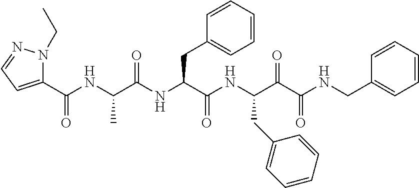 [SMILES: CCn1nccc1C(=O)N[C@@H](C)C(=O)N[C@@H](Cc1ccccc1)C(=O)N[C@@H](Cc1ccccc1)C(=O)C(=O)NCc1ccccc1]

To a solution of (S)-3-[(S)-2-((S)-2-amino-propionylamino)-3-phenyl-propionylamino]-N-benzyl-2-oxo-4-phenyl-butyramide trifluoroacetate (60 mg, 0.098 mmol) in DMF (2 ml) was added 1-ethyl-1H-pyrazole-5-carboxylic acid (21 mg, 0.15 mmol), HATU (41 mg, 0.107 mmol) and N,N-diisopropylethylamine (0.085 ml, 0.49 mmol). The reaction mixture was stirred at room temperature for 30 min then quenched with cold water. The resultant precipitate was filtered and washed with water then dried. The crude solid was triturated with Et₂O then purified by chromatography (silica gel, gradient: 0-10% MeOH/dichloromethane) to obtain 15 mg (24%) of 2-ethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide as a light yellow powder. LC/MS: (M+H)⁺=623.

### Example 55

### 2-Methyl-5-trifluoromethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide

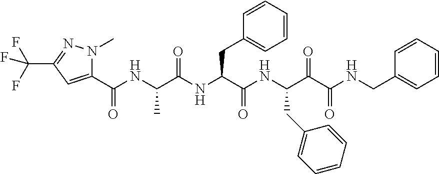 [SMILES: C[C@H](NC(=O)c1cc(C(F)(F)F)nn1C)C(=O)N[C@@H](Cc1ccccc1)C(=O)N[C@@H](Cc1ccccc1)C(=O)C(=O)NCc1ccccc1]

To a solution of (S)-3-[(S)-2-((S)-2-amino-propionylamino)-3-phenyl-propionylamino]-N-benzyl-2-oxo-4-phenyl-butyramide trifluoroacetate (60 mg, 0.098 mmol) in DMF (2 ml) was added 1-methyl-3-(trifluoromethyl)-1H-pyrazole-5-carboxylic acid (20 mg, 0.103 mmol), HATU (41 mg, 0.107 mmol) and N,N-diisopropylethylamine (0.085 ml, 0.49 mmol). The reaction mixture was stirred at room temperature for 30 min then quenched with cold water. The resultant precipitate was filtered, washed with water and Et₂O and dried to obtain 33 mg (49%) of 2-methyl-5-trifluoromethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide as a light yellow powder. LC/MS: (M+H)⁺=677.

### Example 56

### {(S)-1-[1-((S)-1-Benzyl-2-benzylcarbamoyl-2-oxo-ethylcarbamoyl)-2-(tetrahydro-pyran-4-yl)-ethylcarbamoyl]-ethyl}-carbamic acid benzyl ester

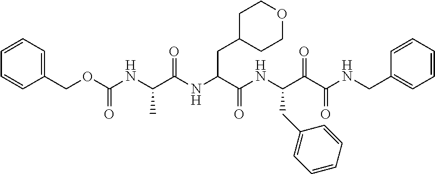 [SMILES: C[C@H](NC(=O)OCc1ccccc1)C(=O)NC(CC1CCOCC1)C(=O)N[C@@H](Cc1ccccc1)C(=O)C(=O)NCc1ccccc1]

To a solution of ((S)-1-benzyl-2-benzylcarbamoyl-2-hydroxy-ethyl)-carbamic acid tert-butyl ester (200 mg, 0.52 mmol) in dichloromethane (5 ml) was added TFA (0.80 ml, 10.4 mmol). The reaction mixture was stirred at room temperature for 2 h then concentrated, chased with hexanes and dried under high vacuum. The residue was dissolved in DMF (2 ml) and 2-tert-butoxycarbonylamino-3-(tetrahydro-pyran-4-yl)-propionic acid (149 mg, 0.55 mmol), HATU (218 mg, 0.57 mmol), and N,N-diisopropylethylamine (0.91 ml, 5.2 mmol) were added. The reaction mixture was stirred at room temperature for 2 h then quenched with water and extracted with EtOAc. The organics were dried over MgSO₄ and concentrated to afford 251 mg (89%) of [1-((S)-1-benzyl-2-benzylcarbamoyl-2-hydroxy-ethylcarbamoyl)-2-(tetrahydro-pyran-4-yl)-ethyl]-carbamic acid tert-butyl ester as an epimeric mixture which was used without further purification.

To a solution of [1-((S)-1-benzyl-2-benzylcarbamoyl-2-hydroxy-ethylcarbamoyl)-2-(tetrahydro-pyran-4-yl)-ethyl]-carbamic acid tert-butyl ester (270 mg, 0.50 mmol) in dichloromethane (10 ml) was added TFA (0.77 ml, 10.0 mmol). The reaction mixture was stirred at room temperature for 2 h then concentrated and dried under high vacuum. The residue was dissolved in DMF (4 ml) and (S)-2-(benzyloxycarbonylamino)propanoic acid (123 mg, 0.55 mmol), HATU (209 mg, 0.55 mmol), and N,N-diisopropylethylamine (0.44 ml, 2.5 mmol) were added. The reaction mixture was stirred at room temperature for 2 h then quenched with water. The resultant precipitate was filtered, washed with water, and dried. The crude solid was triturated with Et₂O then purified by chromatography (silica gel, gradient: 0-5% MeOH/dichloromethane) to obtain 270 mg (84%) of {(S)-1-[1-((S)-1-benzyl-2-benzylcarbamoyl-2-hydroxy-ethylcarbamoyl)-2-(tetrahydro-pyran-4-yl)-ethylcarbamoyl]-ethyl}-carbamic acid benzyl ester.

To a solution of {(S)-1-[1-((S)-1-benzyl-2-benzylcarbamoyl-2-hydroxy-ethylcarbamoyl)-2-(tetrahydro-pyran-4-yl)-ethylcarbamoyl]-ethyl}-carbamic acid benzyl ester (270 mg, 0.42 mmol) in dichloromethane (10 ml) at room temperature was added Dess-Martin periodinane (195 mg, 0.46 mmol). The reaction mixture was stirred at room temperature overnight then quenched with 10% aq. Na₂S₂O₃ and satd. NaHCO₃. The biphasic mixture was stirred vigorously for 30 min then diluted with water and extracted with dichloromethane. The organic phase was washed with satd. NaHCO₃ then dried over MgSO₄ and concentrated. The residue was purified by chromatography (silica gel, gradient: 0-5% MeOH/dichloromethane) to obtain 80 mg (30%) of {(S)-1-[1-((S)-1-benzyl-2-benzylcarbamoyl-2-oxo-ethylcarbamoyl)-2-(tetrahydro-pyran-4-yl)-ethylcarbamoyl]-ethyl}-carbamic acid benzyl ester as a light yellow powder. LC/MS: (M+H)⁺=643.

### Example 57

### {(S)-1-[1-((S)-1-Benzyl-2-benzylcarbamoyl-2-oxo-ethylcarbamoyl)-3,3,3-trifluoro-propylcarbamoyl]-ethyl}-carbamic acid benzyl ester

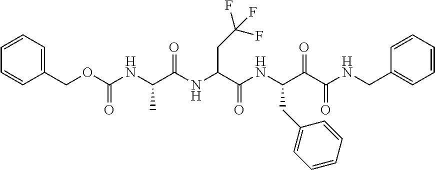 [SMILES: C[C@H](NC(=O)OCc1ccccc1)C(=O)NC(CC(F)(F)F)C(=O)N[C@@H](Cc1ccccc1)C(=O)C(=O)NCc1ccccc1]

To a solution of (S)-3-amino-N-benzyl-2-hydroxy-4-phenyl-butyramide trifluoroacetate (98 mg, 0.25 mmol) in DMF (1 ml) were added (S)-2-(tert-butoxycarbonylamino)-4,4,4-trifluorobutanoic acid (70 mg, 0.27 mmol), HATU (103 mg, 0.27 mmol), and N,N-diisopropylethylamine (0.22 ml, 1.23 mmol). The reaction mixture was stirred at room temperature for 3 h then quenched with water. The resultant precipitate was filtered and dried. The crude solid was triturated with Et₂O to obtain 91 mg (71%) of [(S)-1-((S)-1-benzyl-2-benzylcarbamoyl-2-hydroxy-ethylcarbamoyl)-3,3,3-trifluoro-propyl]-carbamic acid tert-butyl ester as an off-white powder.

To a solution of [(S)-1-((S)-1-benzyl-2-benzylcarbamoyl-2-hydroxy-ethylcarbamoyl)-3,3,3-trifluoro-propyl]-carbamic acid tert-butyl ester (85 mg, 0.16 mmol) in dichloromethane (5 ml) was added TFA (0.25 ml, 3.25 mmol). The reaction mixture was stirred at room temperature for 16 h then concentrated and dried under high vacuum. The residue was dissolved in DMF (1 ml) and (S)-2-(benzyloxycarbonylamino)propanoic acid (40 mg, 0.18 mmol), HATU (68 mg, 0.18 mmol), and N,N-diisopropylethylamine (0.14 ml, 0.81 mmol) were added. The reaction mixture was stirred at room temperature for 1 h then quenched with water. The resultant precipitate was filtered, washed with water and dried. The crude solid was triturated with Et₂O to obtain 95 mg (93%) of {(S)-1-[1-((S)-1-benzyl-2-benzylcarbamoyl-2-hydroxy-ethylcarbamoyl)-3,3,3-trifluoro-propylcarbamoyl]-ethyl}-carbamic acid benzyl ester as an off-white powder.

To a solution of {(S)-1-[1-((S)-1-benzyl-2-benzylcarbamoyl-2-hydroxy-ethylcarbamoyl)-3,3,3-trifluoro-propylcarbamoyl]-ethyl}-carbamic acid benzyl ester (82 mg, 0.13 mmol) in dichloromethane (5 ml) at room temperature was added Dess-Martin periodinane (61 mg, 0.14 mmol). The reaction mixture was stirred at room temperature overnight then quenched with 10% aq. Na₂S₂O₃ and satd. NaHCO₃. The biphasic mixture was stirred vigorously for 30 min then diluted with water and extracted with dichloromethane. The organics were washed with satd. NaHCO₃ then dried over MgSO₄ and concentrated. The residue was triturated with Et₂O to afford 28 mg (34%) of {(S)-1-[1-((S)-1-benzyl-2-benzylcarbamoyl-2-oxo-ethylcarbamoyl)-3,3,3-trifluoro-propylcarbamoyl]-ethyl}-carbamic acid benzyl ester as a white powder. (M+H)⁺=627.

### Example 58

### {(S)-1-[(S)-1-((S)-1-Benzyl-2-benzylcarbamoyl-2-oxo-ethylcarbamoyl)-2-pyridin-4-yl-ethylcarbamoyl]-ethyl}-carbamic acid benzyl ester

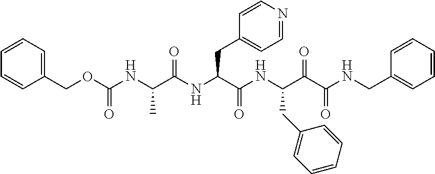 [SMILES: C[C@H](NC(=O)OCc1ccccc1)C(=O)N[C@@H](Cc1ccncc1)C(=O)N[C@@H](Cc1ccccc1)C(=O)C(=O)NCc1ccccc1]

To a solution of ((S)-1-benzyl-2-benzylcarbamoyl-2-hydroxy-ethyl)-carbamic acid tert-butyl ester (541 mg, 1.41 mmol) in dichloromethane (5 ml) was added TFA (2.17 ml, 28.1 mmol). The reaction mixture was stirred at room temperature for 4 h then concentrated, chased with hexanes and dried under high vacuum. The residue was dissolved in DMF (2 ml) and (S)-2-(tert-butoxycarbonylamino)-3-(pyridin-4-yl)propanoic acid (375 mg, 1.41 mmol), HATU (589 mg, 1.55 mmol) and N,N-diisopropylethylamine (2.46 ml, 14.1 mmol) were added. The reaction mixture was stirred at room temperature for 16 h then quenched with water. The resultant precipitate was filtered, washed with water, and dried. The crude solid was triturated with Et₂O to provide 710 mg (95%) of [(S)-1-((S)-1-benzyl-2-benzylcarbamoyl-2-hydroxy-ethylcarbamoyl)-2-pyridin-4-yl-ethyl]-carbamic acid tert-butyl ester as a white powder.

To a solution of [(S)-1-((S)-1-benzyl-2-benzylcarbamoyl-2-hydroxy-ethylcarbamoyl)-2-pyridin-4-yl-ethyl]-carbamic acid tert-butyl ester (710 mg, 1.33 mmol) in dichloromethane (10 ml) was added TFA (2.05 ml, 26.7 mmol). The reaction mixture was stirred at room temperature for 4 h then concentrated, chased with hexanes, and dried under high vacuum. The residue was dissolved in DMF (5.0 ml) and (S)-2-(benzyloxycarbonylamino)propanoic acid (327 mg, 1.47 mmol), HATU (558 mg, 1.47 mmol) and N,N-diisopropylethylamine (1.16 ml, 6.67 mmol) were added. The reaction mixture was stirred at room temperature for 16 h then quenched with water. The resultant precipitate was filtered and washed with water then dried. The crude solid was triturated with Et₂O to obtain 750 mg (88%) of {(S)-1-[(S)-1-((S)-1-benzyl-2-benzylcarbamoyl-2-hydroxy-ethylcarbamoyl)-2-pyridin-4-yl-ethylcarbamoyl]-ethyl}-carbamic acid benzyl ester as an off-white powder.

To a solution of {(S)-1-[(S)-1-((S)-1-benzyl-2-benzylcarbamoyl-2-hydroxy-ethylcarbamoyl)-2-pyridin-4-yl-ethylcarbamoyl]-ethyl}-carbamic acid benzyl ester (400 mg, 0.63 mmol) in dichloromethane (10 ml) at room temperature was added Dess-Martin periodinane (293 mg, 0.69 mmol). The reaction mixture was stirred at room temperature overnight then quenched with 10% aq. Na₂S₂O₃ and satd. NaHCO₃. The biphasic mixture was stirred vigorously for 30 min then diluted with water and extracted with dichloromethane. The organics were washed with satd. NaHCO₃ then dried over MgSO₄ and concentrated. The residue was chromatographed (silica gel, gradient: 2-10% MeOH/dichloromethane) to give 49 mg (11%) of {(S)-1-[(S)-1-((S)-1-Benzyl-2-benzylcarbamoyl-2-oxo-ethylcarbamoyl)-2-pyridin-4-yl-ethylcarbamoyl]-ethyl}-carbamic acid benzyl ester as a white powder. LC/MS: (M+H)⁺=636.

### Example 59

### Indan-2-carboxylic acid {(S)-1-[(S)-1-(2-benzylaminooxalyl-indan-2-ylcarbamoyl)-2-(4-methoxy-phenyl)-ethylcarbamoyl]-ethyl}-amide

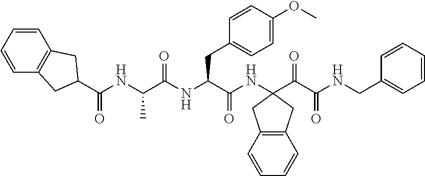 [SMILES: COc1ccc(C[C@H](NC(=O)[C@H](C)NC(=O)C2Cc3ccccc3C2)C(=O)NC2(C(=O)C(=O)NCc3ccccc3)Cc3ccccc3C2)cc1]

In a 50 ml round-bottomed flask, 2-tert-butoxycarbonylamino-indan-2-carboxylic acid (940 mg, 3.39 mmol) and N,O-dimethylhydroxylamine hydrochloride (496 mg, 5.08 mmol) were dissolved in 9.0 ml DMF. N,N-Diisopropylethylamine (1.26 g, 1.7 ml, 9.76 mmol) was added followed by HATU (1.42 g, 3.73 mmol). The yellow solution was stirred at room temperature overnight. The reaction mixture was quenched with 10 ml water and extracted twice with 70 ml diethyl ether. The organic layers were washed twice with 10 ml water and once with 10 ml brine. The organic layers were combined, dried over sodium sulfate, filtered and concentrated. The residue was chromatographed over 40 g silica gel with EtOAc/hexanes (gradient 0-30% EtOAc). All fractions containing product were combined and concentrated to provide 1.03 g (95%) [2-(methoxy-methyl-carbamoyl)-indan-2-yl]-carbamic acid tert-butyl ester as an off-white foam.

In a 100 ml 2-neck round-bottomed flask, [2-(methoxy-methyl-carbamoyl)-indan-2-yl]-carbamic acid tert-butyl ester (1.025 g, 3.2 mmol) was dissolved in 12 ml THF. The colorless solution was cooled to 0° C. Lithium aluminum hydride, 1.0M solution in THF (3.4 ml, 3.4 mmol) was added dropwise at 0° C. (gas evolution observed). The reaction mixture was stirred at 0° C. for 1 h. Sodium sulfate decahydrate was added carefully to quench the reaction. When no more gas evolution was detected, the ice bath was removed, sodium sulfate and ethyl acetate were added and the mixture was stirred vigorously for 30 min at room temperature. The suspension was filtered over filter paper and rinsed with ethyl acetate. The filtrate was concentrated to afford (2-formyl-indan-2-yl)-carbamic acid tert-butyl ester as a colorless oil which was used immediately without further purification.

In a 50 ml round-bottomed flask, (2-formyl-indan-2-yl)-carbamic acid tert-butyl ester (931 mg, 3.03 mmol; purity=85%) was dissolved in 9.5 ml dichloromethane. Acetone cyanohydrin (781 mg, 0.84 ml, 9.18 mmol) was added followed by triethylamine (203 mg, 0.28 ml, 2.01 mmol). The reaction mixture was stirred at room temperature for 4 h. The reaction mixture was extracted with 70 ml diethyl ether and 10 ml water. The aqueous layer was extracted with 70 ml diethyl ether. The organic layers were washed four times with 10 ml water and once with 10 ml brine. The organic layers were combined, dried over sodium sulfate, filtered and concentrated to afford 846 mg (97%) [2-(cyano-hydroxy-methyl)-indan-2-yl]-carbamic acid tert-butyl ester as an off-white solid which was used without further purification.

A 100 ml round-bottomed flask was charged with [2-(cyano-hydroxy-methyl)-indan-2-yl]-carbamic acid tert-butyl ester (0.844 g, 2.93 mmol) and 6M hydrochloric acid (13 ml, 78.0 mmol). The reaction mixture was stirred at 100° C. overnight. The reaction mixture was cooled to room temperature, concentrated on rotavap and then dried under high vacuum to give a sticky brown solid. The residue was dissolved in 5.2 ml 1,4-dioxane and 5.2 ml water. Sodium bicarbonate (2.46 g, 29.3 mmol) was added followed by di-tert-butyl dicarbonate (958 mg, 4.39 mmol). The heterogeneous light brown reaction mixture was stirred vigorously at room temperature for two days. The reaction mixture was diluted with 15 ml water and extracted with 70 ml diethyl ether. The organic layer was washed twice with 5 ml water. The organic layer was set aside and later discarded. The aqueous layers were combined, acidified with conc. HCl until pH=˜2 and then extracted three times with 70 ml diethyl ether. The organic layers were combined, dried over sodium sulfate, filtered and concentrated to afford 229 mg (25%) (2-tert-butoxycarbonylamino-indan-2-yl)-hydroxy-acetic acid as a light yellow solid.

In a 50 ml round-bottomed flask, (2-tert-butoxycarbonylamino-indan-2-yl)-hydroxy-acetic acid (226 mg, 0.74 mmol) was dissolved in 2.5 ml DMF. Benzylamine (98.1 mg, 0.10 ml, 0.92 mmol) was added followed by N,N-Diisopropylethylamine (200 mg, 0.27 ml, 1.55 mmol) and HATU (308 mg, 0.81 mmol). The yellow solution was stirred at room temperature for five days. The reaction mixture was extracted with EtOAc and water. The aqueous layer was back extracted with EtOAc. The organic layers were washed twice with water and once with brine. The organic layers were combined, dried over sodium sulfate, filtered and concentrated. The residue was chromatographed over 25 g silica gel with EtOAc/hexanes (gradient 0-20% EtOAc). All fractions containing product were combined and concentrated to provide 95 mg (33%) [2-(benzylcarbamoyl-hydroxy-methyl)-indan-2-yl]-carbamic acid tert-butyl ester as an off-white solid.

In a 25 ml round-bottomed flask, [2-(benzylcarbamoyl-hydroxy-methyl)-indan-2-yl]-carbamic acid tert-butyl ester (95 mg, 0.24 mmol) was dissolved in 2.7 ml dichloromethane. Trifluoroacetic acid (0.47 ml, 6.1 mmol) was added slowly. The reaction mixture was stirred at room temperature for 2 h. The solvent was evaporated and then placed under high vacuum for 15 min. The residue and (S)-2-{(S)-2-[(indane-2-carbonyl)-amino]-propionylamino}-3-(4-methoxy-phenyl)-propionic acid (100 mg, 0.23 mmol) were dissolved in 1.8 ml DMF and cooled to 0° C. N,N-Diisopropylethylamine (178 mg, 0.24 ml, 1.37 mmol) was added dropwise at 0° C. followed by HATU (97 mg, 0.26 mmol). After the addition was complete, the ice bath was removed and the reaction mixture was stirred at room temperature for two days. The reaction mixture was extracted with EtOAc and water. The aqueous layer was back extracted with EtOAc. The organic layers were washed three times with water and once with brine. The organic layers were combined, dried over sodium sulfate, filtered and concentrated. The residue was chromatographed over 12 g silica gel with MeOH/dichloromethane (gradient 0-5% MeOH). All fractions containing product were combined and concentrated to afford 159 mg (70%; purity=90%) indan-2-carboxylic acid {(S)-1-[(S)-1-[2-(benzylcarbamoyl-hydroxy-methyl)-indan-2-ylcarbamoyl]-2-(4-methoxy-phenyl)-ethylcarbamoyl]-ethyl}-amide as a colorless oil.

In a 50 ml round-bottomed flask, indan-2-carboxylic acid {(S)-1-[(S)-1-[2-(benzylcarbamoyl-hydroxy-methyl)-indan-2-ylcarbamoyl]-2-(4-methoxy-phenyl)-ethylcarbamoyl]-ethyl}-amide (121 mg, 0.16 mmol; purity=90%) was partially dissolved in 6.5 ml dichloromethane and Dess-Martin periodinane (101 mg, 0.24 mmol) was added. The reaction mixture was stirred at room temperature for 2 h. The reaction mixture was quenched with 3 ml saturated NaHCO₃-solution and 3 ml 10% Na₂S₂O₃-solution and stirred vigorously for 15 min at room temperature. The biphasic mixture was then extracted with 30 ml dichloromethane. The organic layer was washed with 10 ml saturated NaHCO₃-solution. The aqueous layers were extracted twice with 30 ml dichloromethane. The organic layers were combined, dried over sodium sulfate, filtered and concentrated. The residue was triturated with diethyl ether/ethyl acetate to afford 61 mg of an off-white solid. The residue was chromatographed over 12 g silica gel with MeOH/dichloromethane (gradient 0-5% MeOH). All fractions containing product were combined and concentrated to afford 39 mg (34%) indan-2-carboxylic acid {(S)-1-[(S)-1-(2-benzylaminooxalyl-indan-2-ylcarbamoyl)-2-(4-methoxy-phenyl)-ethylcarbamoyl]-ethyl}-amide as an off-white solid. LC/MS: (M−H)⁻=685.

### Example 60

### Assay Protocols and Results

Cell-Based Proteasome Activity/Selectivity Assay

The Cell-Based Proteasome subunit activity/selectivity assay was a panel of 5 fluorogenic assays that independently measured the activity of β5c or β5i (chymotrypsin-like activity), β2c/2i (trypsin-like), and β1c or β1i (caspase-like) protease activity associated with the proteasome complex in cultured cells. Specifically, the following substrates were used for respective subunit activities: β1i: (PAL)₂Rh110, β1c: (LLE)₂ Rh110, β2c/2i: (KQL)₂Rh110, β5c: (WLA)₂Rh110, β5i: (ANW)₂Rh110. The following procedure was followed:

Cell preparation: Plated 25 μl of Ramos cells (2×10⁶/ml in DPBS) into half area plate (PerkinElmer Cat 6005569) to final 5×10⁴ cells/well. Added 0.5 μl of 100×4-fold serial diluted test compounds or DMSO to each well. Highest concentration of compound tested was 20 μM, thus compound serial dilution started from 200 mM. Incubated for 30 minutes at 37° C. Then equilibrated at room temperature for 15 minutes. Added 25 μl of 2× reaction mix consisting of 0.025% digitonin, 20 μM of each substrates and 0.5M sucrose in DPBS. Shaked for one minute @ 700 rpm. Incubated for 120 min at room temperature. Then read the plates with an Envision multilabel plate reader (PerkinElmer) with 500 nm excitation/519 nm emission.

Modified PBMC Proteasome Activity Assay

This cell-based proteasome activity assay was similar to previous Ramos cell-based assay as of the substrates, but using human PBMCs in the context of complete RPMI with 10% FBS as reaction buffer. This assay was designed to assess the level of cellular penetration of test compounds in primary human cells. The following procedure was followed: Fresh isolated PBMC from healthy donor were plated at 1×10⁵ cells/well in 100 μl of complete RPMI with 10% FBS in V bottom 96 plates. Added 1 μl of 100×4-fold serial diluted compounds/well and incubated for 1 hr. The highest compound concentration tested was 20 μM (100× working stock start with 2 mM). Spun down the cells @ 2000 rpm for 5 min. Removed all supernatant. Then resuspended the cells in 25 μl DPBS and transferred the cells to a fresh half-area plate (PerkinElmer Cat 6005569). In the final reaction volume was 50 μl, including 25 μl cell suspension, 0.5 μl 100× inhibitor or DMSO, 25 μl substrate mix containing 0.025% digitonin, 20 uM substrate (Substrate: (PAL)₂Rh110, (LLE)₂ Rh110, (KQL)₂Rh110, (WLA)₂Rh110, or (ANW)₂Rh110)/in 10% FBS and 0.5M sucrose mixture. Shaked for one minute (@ 700 rpm). Incubated for 2 hrs, then read the plates with Envision plate reader using 500 nm excitation/519 nm emission.

PBMC IP-10 Assay

PBMCs were isolated from whole blood as follows: Blood was collected in a sterile environment in heparinized tubes. Blood was diluted with an equal volume PBS/2% FCS and 30 ml of this mixture was added to ACCUSPIN tubes containing 15 ml Histopaque-1077 already centrifuged at 800 g for 30 seconds and warmed up at room temperature. The tubes were then centrifuged at 800 g for 20 minutes at room temperature with no brake. The mononuclear band, just above the polyethylene frit, was removed by Pasteur pipet. These mononuclear cells were washed three times with sterile PBS, counted, and resuspended in RPMI 1640 supplemented with 10% heat inactivated fetal calf serum, 10 mM HEPES, 1 mM sodium pyruvate, penicillin (50 U/ml), streptomycin (50 μg/ml) and glutamine (2 mM) to approximately 1.5×10⁶/ml. Approximately 2×10⁵ cells/well were plated in 96 well tissue culture plates (BD Falcon 353072), and preincubated 60 mi/37° C. with a titration of compounds, in a final concentration of 1% DMSO. Cells were then stimulated with CpG Type A (Invivogen, Cat #tlrl-2216; ODN 2216) at a final concentration of 2.5 μM. Cells were incubated overnight, and supernatants were removed. PBMC viability of cells remaining in the well was measured with ATPlite luminescence assay (Perkin-Elmer) per the manufacturer's instructions. Luminescence was measured on the Perkin-Elmer Envision, using the luminescence filter. IP10 level was measured with CXCL10/IP10 AlphaLISA kit (Perkin-Elmer) per the manufacturer's instructions, except halving all volumes. Fluorescence was measured on the Envision Multilabel plate reader, using the AlphaScreen standard settings.

Results:

The results of the above assays for representative compounds of the invention are provided in Table 1 below, wherein the IC50 and EC50 activity values are in μM:

TABLE 1

| | Ic50 | Ic50 | Ic50 | Ic50 | Ic50 | |
|---|---|---|---|---|---|---|
| Example | ramos:ac- | ramos:rh110- | ramos:rh110- | ramos:rh110- | ramos:rh110- | |
| No. | (anw)2-r110 | (wla)2 | (kql)2 | (pal)2 | (lle)2 | Ec50 |
| 1 | 0.146 | 0.45 | 10.568 | 0.91 | 3.546 | 2.0485 |
| 2 | 2.079 | 2.431 | 20 | 0.661 | 8.7 | 12.139 |
| 3 | 0.394 | 3.2 | 20 | 0.56 | 20 | 7.959 |
| 4 | 0.083 | 0.763 | 20 | 0.091 | 5.698 | 3.9135 |
| 5 | 0.018 | 0.56 | 20 | 0.142 | 3.345 | 1.216 |
| 6 | 1.04 | 5.518 | 20 | 0.949 | 20 | 13.569 |
| 7 | 0.102 | 3.048 | 20 | 0.712 | 13.183 | 4.008 |
| 8 | 0.033 | 0.771 | 20 | 0.504 | 11.097 | 1.783 |
| 9 | 0.026 | 0.815 | 20 | 0.034 | 20 | 5.287 |
| 10 | 0.709 | 1.904 | 20 | 0.275 | 20 | |
| 11 | 0.014 | 0.473 | 8.035 | | 2.984 | 1.224 |
| 12 | 0.036 | 0.381 | 20 | | 5.434 | 2.612 |
| 13 | 0.403 | 2.961 | 20 | | 9.56 | 3.974 |
| 14 | 0.699 | 16.545 | 20 | 0.426 | 20 | 5.175 |
| 15 | 0.101 | 3.538 | 20 | 0.21 | 8.428 | 1.912 |
| 16 | 0.017 | 0.926 | 20 | 0.274 | 18.332 | 1.331 |
| 17 | 0.007 | 0.988 | 20 | 0.417 | 20 | 0.939 |
| 18 | 0.005 | 0.173 | 7.023 | 0.441 | 3.275 | 0.672 |
| 19 | 0.003 | 0.055 | 20 | 0.234 | 20 | 0.695 |
| 20 | 0.015 | 0.157 | 20 | 0.738 | 20 | 1.635 |
| 21 | 0.007 | 0.765 | 20 | 0.21 | 20 | 3.486 |
| 22 | 0.124 | 3.298 | 20 | 1.095 | 20 | 5.777 |
| 23 | 0.001 | 0.005 | 20 | 0.097 | 7.324 | 0.356 |
| 24 | 0.005 | 0.272 | 5.71 | 0.441 | 7.048 | 0.433 |
| 25 | 0.009 | 0.846 | 20 | 20 | 20 | 1.075 |
| 26 | 0.004 | 0.015 | 0.38 | 0.181 | 2.384 | 0.203 |
| 27 | 0.04 | 4.52 | 20 | 5.061 | 12.945 | 4.87 |
| 28 | 0.294 | 5.114 | 20 | 4.74 | 20 | 2.793 |
| 29 | 0.275 | 2.056 | 11.947 | 0.218 | 5.622 | 4.167 |
| 30 | 0.047 | 2.476 | 10.041 | 0.569 | 12.519 | 4.089 |
| 31 | 0.034 | 0.738 | 20 | 0.244 | 13.067 | 1.179 |
| 32 | 0.011 | 0.365 | 20 | 0.266 | 12.172 | 0.94 |
| 33 | 0.014 | 0.597 | 20 | 0.205 | 17.513 | 1.382 |
| 34 | 0.01 | 1.092 | 20 | 0.589 | 13.51 | 1.163 |
| 35 | 0.004 | 0.261 | 20 | 0.395 | 13.394 | 0.615 |
| 36 | 0.062 | 0.973 | 20 | 0.359 | 20 | 2.796 |
| 37 | 0.234 | 4.442 | 20 | 1.081 | 20 | 5.103 |
| 38 | 1.21 | 4.113 | 20 | 20 | 20 | 10.116 |
| 39 | 0.029 | 1.379 | 20 | 0.605 | 20 | 2.316 |
| 40 | 0.046 | 1.292 | 20 | 1.144 | 14.746 | 1.833 |
| 41 | 0.026 | 0.385 | 20 | 0.697 | 18.41 | 1.495 |
| 42 | 0.004 | 0.014 | 20 | 0.968 | 18.085 | 0.379 |
| 43 | 0.003 | 0.192 | 20 | 0.257 | 18.262 | 0.784 |
| 44 | 0.013 | 1.238 | 20 | 13.095 | 20 | 0.596 |
| 45 | 0.013 | 0.25 | 20 | 0.496 | 6.38 | 1.318 |
| 46 | 0.121 | 4.414 | 20 | 2.171 | 20 | 5.781 |
| 47 | 0.034 | 1.392 | 20 | 0.705 | 6.267 | 2.292 |
| 48 | 0.008 | 1.013 | 20 | 1.169 | 13.638 | 0.615 |
| 49 | 0.105 | 1.927 | 20 | 0.499 | 17.471 | 4.374 |
| 50 | 0.266 | 2.886 | 20 | 1.277 | 20 | 2.74 |
| 51 | 0.021 | 5.185 | 20 | 9.842 | 20 | 2.588 |
| 52 | 0.221 | 2.351 | 20 | 1.531 | 10.259 | 4.505 |
| 53 | 0.221 | 1.365 | 20 | 1.78 | 20 | 4.898 |
| 54 | 0.066 | 1.683 | 20 | 0.609 | 18.504 | 3.453 |
| 55 | 0.176 | 5.768 | 20 | 8.919 | 20 | 3.885 |
| 56 | 0.115 | 3.116 | 20 | 0.879 | 20 | 3.234 |
| 57 | 0.524 | 6.164 | 20 | 2.729 | 20 | 5.030 |
| 58 | 0.03 | 0.96 | 20 | 0.478 | 20 | 1.331 |
| 59 | 0.089 | 1.36 | 20 | 20 | 20 | 11.67 |

It is to be understood that the invention is not limited to the particular embodiments of the invention described above, as variations of the particular embodiments may be made and still fall within the scope of the appended claims.

1. A compound of formula (I): 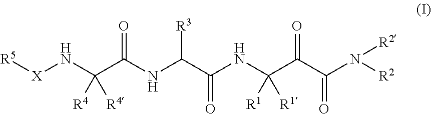
wherein:
X is —C(O)— or —S(O)₂—;
one of R¹ or R^{1′} is H or unsubstituted C₁₋₇ alkyl and the other is unsubstituted C₁₋₇ alkyl or C₁₋₇ alkyl substituted with phenyl, or
R¹ and R^{1′}, together with the carbon atom to which they are attached, combine to form an indanyl moiety;
one of R² or R^{2′} is H or methyl and the other is cycloalkyl, unsubstituted C₁₋₇ alkyl, or C₁₋₇ alkyl substituted with phenyl, alkoxy or heteroaryl;
R³ is unsubstituted C₁₋₇ alkyl or C₁₋₇ alkyl substituted with phenyl, methoxyphenyl, indolyl, alkoxy, —SO₂CH₃, heteroaryl, chlorophenyl, heterocycle or CF₃;
one of R⁴ or R^{4′} is H or unsubstituted C₁₋₇ alkyl and the other is unsubstituted C₁₋₇ alkyl or C₁₋₇ alkyl substituted with alkoxy or cycloalkyl, or
R⁴ or R^{4′}, together with the carbon atom to which they are attached, combine to form a cycloalkyl moiety;
R⁵ is selected from:
CH₃C(O)NHCH(CH₂-phenylmethyl),
isoindolyl,
dihydroisoindolyl,
—CH₂-heterocycle,
—CH₂-heteroaryl,
—CH₂—CH₂-methylpyrazolyl,
methyl-indenyl,
—CH₂-phenyl,
indanyl,
methyl-isoxazolyl,
unsubstituted heteroaryl,
heteroaryl mono- or bi-substituted independently with C₁₋₇ alkyl or CF₃,
unsubstituted phenyl,
phenyl mono- or bi-substituted independently with C₁₋₇ alkyl or halogen,
—CH₂-benzo[1,4]oxazinyl,
—CH₂-dihydrobenzo[1,4]oxazinyl,
—O—CH₂-phenyl,
methyl-indolyl,
methyl-pyrrolo[3,2-b]pyridinyl or
imidazo[1,2-a]pyridinyl,
or a pharmaceutically acceptable salt thereof.

2. The compound according to claim 1, wherein X is —C(O)—.

3. The compound according to claim 1, wherein one of R¹ or R^{1′} is H and the other is butyl or —CH₂-phenyl.

4. The compound according to claim 1, wherein one of R1 or R1′ is H and the other is —CH₂-phenyl.

5. The compound according to claim 1, wherein one of R² or R^{2′} is H and the other is cyclopropyl, methyl, —CH₂-phenyl, —CH₂—CH₂-phenyl, —CH₂CH₂OCH₃ or —CH₂-pyridinyl.

6. The compound according to claim 1, wherein R² or R^{2′} is H and the other is —CH₂-phenyl.

7. The compound according to claim 1, wherein R³ is tert-butyl, iso-butyl, —CH₂-phenyl, —CH₂-methoxyphenyl, —CH₂-indolyl, —CH₂-methoxy, —CH₂CH₂SO₂CH₃, —CH₂-pyranyl, —CH₂-pyridinyl, —CH₂-chlorophenyl, —CH₂-tetrahydropyranyl or —CH₂CF₃.

8. The compound according to claim 1, wherein R³ is —CH₂-methoxyphenyl or —CH₂-indolyl.

9. The compound according to claim 1, wherein one of R⁴ or R^{4′} is H and the other is methyl, tert-butyl, —CH₂—OCH₃ or cyclopropyl.

10. The compound according to claim 1, wherein R⁴ or R^{4′}, together with the carbon atom to which they are attached, combine to form a cyclopropyl moiety.

11. The compound according to claim 1, wherein one of R4 or R4′ is H and the other is methyl.

12. The compound according to claim 1, wherein R⁵ is CH₃C(O)NHCH(CH₂-phenylmethyl), -dihydroindolyl, —CH₂-morpholine, —CH₂—CH₂-methylpyrazolyl, methyl-indenyl, —CH₂-phenyl, indanyl, methyl-isoxazolyl, pyrazinyl, methyl-pyrazolyl, dimethyl-pyrazolyl, ethyl-pyrazolyl, methyl-trifluoromethyl-pyrazolyl, phenyl, dichloro-phenyl, methyl-phenyl, —CH₂-benzo[1,4]oxazinyl, —CH₂-dihydrobenzo[1,4]oxazinyl, —O—CH₂-phenyl, methyl-indolyl, methyl-pyrrolo[3,2-b]pyridinyl or imidazo[1,2-a]pyridinyl.

13. The compound according to claim 1, wherein R⁵ is indanyl or —O—CH₂-phenyl.

14. The compound according to claim 1 having a formula (I′): 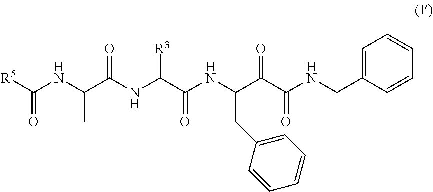
wherein R³ and R⁵ are as defined in claim 1;
or a pharmaceutically acceptable salt thereof.

15. The compound according to claim 14, wherein:
R³ is —CH₂-methoxyphenyl or —CH₂-indolyl,
R⁵ is indanyl or —O—CH₂-phenyl,
or a pharmaceutically acceptable salt thereof.

16. The compound according to claim 1, wherein said compound is:
(S)-3-{(S)-2-[(S)-2-((S)-2-Acetylamino-3-o-tolyl-propionylamino)-3,3-dimethyl-butyrylamino]-4-methyl-pentanoylamino}-2-oxo-heptanoic acid benzylamide;
(S)-3-{(S)-2-[(S)-3,3-Dimethyl-2-(2-morpholin-4-yl-acetylamino)-butyrylamino]-4-methyl-pentanoylamino}-2-oxo-heptanoic acid benzylamide;
(S)-3-(S)-3-(4-M ethoxy-phenyl)-2-[(S)-2-(2-morpholin-4-yl-acetylamino)-propionylamino]-propionylamino)-2-oxo-heptanoic acid benzylamide;
5-Methyl-isoxazole-3-carboxylic acid {(S)-1-[(S)-1-((S)-1-benzylaminooxalyl-pentylcarbamoyl)-2-(1H-indol-3-yl)-ethylcarbamoyl]-ethyl}-amide;
3-Methyl-1H-indene-2-carboxylic acid {(S)-1-[(S)-1-((S)-1-benzylaminooxalyl-pentylcarbamoyl)-2-(1H-indol-3-yl)-ethylcarbamoyl]-ethyl}-amide;
5-Methyl-isoxazole-3-carboxylic acid {(S)-1-[(S)-1-((S)-1-benzylaminooxalyl-pentylcarbamoyl)-2-methoxy-ethylcarbamoyl]-ethyl}-amide;
3-Methyl-1H-indene-2-carboxylic acid ((S)-1-{(S)-2-(1H-indol-3-yl)-1-[(S)-1-((S)-1-phenyl-ethylaminooxalyl)-pentylcarbamoyl]-ethylcarbamoyl}-ethyl)-amide;
3-Methyl-1H-indene-2-carboxylic acid ((S)-1-{(S)-2-(1H-indol-3-yl)-1-[(S)-1-((R)-1-phenyl-ethylaminooxalyl)-pentylcarbamoyl]-ethylcarbamoyl}-ethyl)-amide;
{(S)-1-[(S)-1-((S)-1-Benzylaminooxalyl-pentylcarbamoyl)-2-(1H-indol-3-yl)-ethylcarbamoyl]-ethyl}-carbamic acid benzyl ester;
(S)-3-{(S)-3-(1H-Indol-3-yl)-2-[(S)-2-(2-morpholin-4-yl-acetylamino)-propionylamino]-propionylamino}-2-oxo-heptanoic acid benzylamide;
Indan-2-carboxylic acid {(S)-1-[(S)-1-((S)-1-benzylaminooxalyl-pentylcarbamoyl)-2-(1H-indol-3-yl)-ethylcarbamoyl]-ethyl}-amide;
N-{(S)-1-[(S)-1-((S)-1-Benzylaminooxalyl-pentylcarbamoyl)-2-(1H-indol-3-yl)-ethylcarbamoyl]-ethyl}-2,3-dichloro-benzamide;
2-Methyl-2H-pyrazole-3-carboxylic acid {(S)-1-[(S)-1-((S)-1-benzylaminooxalyl-pentylcarbamoyl)-2-(1H-indol-3-yl)-ethylcarbamoyl]-ethyl}-amide;
(S)-3-(S)-3-(4-Methoxy-phenyl)-2-[(S)-2-(2-morpholin-4-yl-acetylamino)-propionylamino]-propionylamino)-2-oxo-heptanoic acid methylamide;
3-Methyl-1H-indene-2-carboxylic acid {(S)-1-[(S)-1-((S)-1-benzylaminooxalyl-pentylcarbamoyl)-3-methanesulfonyl-propylcarbamoyl]-ethyl}-amide;
{(S)-1-[(S)-1-((S)-1-Benzyl-2-benzylcarbamoyl-2-oxo-ethylcarbamoyl)-2-(1H-indol-3-yl)-ethylcarbamoyl]-ethyl}-carbamic acid benzyl ester;
{(S)-1-[(S)-1-((S)-1-Benzyl-2-benzylcarbamoyl-2-oxo-ethylcarbamoyl)-2-(4-methoxy-phenyl)-ethylcarbamoyl]-ethyl}-carbamic acid benzyl ester;
Indan-2-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide;
Indan-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
3-Methyl-1H-indene-2-carboxylic acid {(S)-1-[(S)-1-((S)-1-benzyl-2-benzylcarbamoyl-2-oxo-ethylcarbamoyl)-3-methanesulfonyl-propylcarbamoyl]-ethyl}-amide;
5-Methyl-isoxazole-3-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
(S)—N-Benzyl-3-{(S)-3-(1H-indol-3-yl)-2-[(S)-2-(2-morpholin-4-yl-acetylamino)-propionylamino]-propionylamino}-2-oxo-4-phenyl-butyramide;
Indan-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-2-methoxy-ethyl}-amide;
Indan-2-carboxylic acid {(S)-1-[(S)-1-((S)-1-benzyl-2-benzylcarbamoyl-2-oxo-ethylcarbamoyl)-3-methyl-butylcarbamoyl]-ethyl}-amide;
3-Methyl-1H-indene-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
Indan-2-carboxylic acid {(S)-[(S)-1-((S)-1-benzyl-2-benzylcarbamoyl-2-oxo-ethylcarbamoyl)-2-(4-methoxy-phenyl)-ethylcarbamoyl]-cyclopropyl-methyl}-amide;
Indan-2-carboxylic acid {1-[(S)-1-((S)-1-benzyl-2-benzylcarbamoyl-2-oxo-ethylcarbamoyl)-2-(4-methoxy-phenyl)-ethylcarbamoyl]-cyclopropyl}-amide;
{1-[(S)-1-((S)-1-Benzyl-2-benzylcarbamoyl-2-oxo-ethylcarbamoyl)-2-(4-methoxy-phenyl)-ethylcarbamoyl]-cyclopropyl}-carbamic acid benzyl ester;
Pyrazine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
2-Methyl-2H-pyrazole-3-carboxylic acid {(S)-1 [(S)-1-((S)-1-benzyl-2-benzylcarbamoyl-2-oxo-ethylcarbamoyl)-2-(1H-indol-3-yl)-ethylcarbamoyl]-ethyl}-amide;
{(S)-1 [(S)-1-((S)-1-Benzyl-2-methylcarbamoyl-2-oxo-ethyl carbamoyl)-2-(1H-indol-3-yl)-ethylcarbamoyl]-ethyl}-carbamic acid benzyl ester;
{(S)-1-[(S)-1-((S)-1-Benzyl-2-cyclopropylcarbamoyl-2-oxo-ethylcarbamoyl)-2-(1H-indol-3-yl)-ethylcarbamoyl]-ethyl}-carbamic acid benzyl ester;
{(S)-1-[(S)-1-[(S)-1-Benzyl-2-(2-methoxy-ethylcarbamoyl)-2-oxo-ethylcarbamoyl]-2-(1H-indol-3-yl)-ethylcarbamoyl]-ethyl}-carbamic acid benzyl ester;
{(S)-1-[(S)-1-(S)-1-Benzyl-2-oxo-2-[(pyridin-2-ylmethyl)-carbamoyl]-ethylcarbamoyl-2-(1H-indol-3-yl)-ethylcarbamoyl]-ethyl}-carbamic acid benzyl ester;
(S)—N-Benzyl-3-[(S)-2-[(S)-2-(2-2,3-dihydro-benzo[1,4]oxazin-4-yl-acetylamino)-propionylamino]-3-(4-methoxy-phenyl)-propionylamino]-2-oxo-4-phenyl-butyramide;
{(S)-1-[(S)-1-((S)-1-Benzyl-2-benzylcarbamoyl-2-oxo-ethylcarbamoyl)-3-methyl-butylcarbamoyl]-ethyl}-carbamic acid benzyl ester;
2-Methyl-2H-pyrazole-3-carboxylic acid {(S)-1-[(s)-1-((S)-1-benzyl-2-benzylcarbamoyl-2-oxo-ethylcarbamoyl)-3-methyl-butylcarbamoyl]-ethyl}-amide;
{(S)-1-[(S)-1-[(S)-1-Benzyl-2-(benzyl-methyl-carbamoyl)-2-oxo-ethylcarbamoyl]-2-(1H-indol-3-yl)-ethylcarbamoyl]-ethyl}-carbamic acid benzyl ester;
(S)-3-[(S)-2-((S)-2-Benzenesulfonylamino-propionylamino)-3-(4-methoxy-phenyl)-propionylamino]-N-benzyl-2-oxo-4-phenyl-butyramide;
(S)—N-Benzyl-3-{(S)-3-(4-methoxy-phenyl)-2-[(S)-2-(toluene-2-sulfonylamino)-propionylamino]-propionylamino}-2-oxo-4-phenyl-butyramide;
(S)—N-Benzyl-3-((S)-3-(4-methoxy-phenyl)-2-{(S)-2-[3-(2-methyl-2H-pyrazol-3-yl)-propionyl amino]-propionylamino}-propionylamino)-2-oxo-4-phenyl-butyramide;
Indan-2-carboxylic acid {(S)-1-[(S)-1-((S)-1-benzyl-2-benzylcarbamoyl-2-oxo-ethyl carbamoyl)-3-phenyl-propylcarbamoyl]-ethyl}-amide;
Indan-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
1-Methyl-1H-indole-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
1-Methyl-1H-pyrrolo[3,2-b]pyridine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
(S)—N-Benzyl-3-{(S)-3-(4-methoxy-phenyl)-2-[(S)-2-(2-morpholin-4-yl-acetylamino)-propionylamino]-propionylamino}-2-oxo-4-phenyl-butyramide;
5-Methyl-isoxazole-3-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide;
3-Methyl-1H-indene-2-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide;
Imidazo[1,2-a]pyridine-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide;
{(S)-1-[(S)-1-((S)-1-Benzyl-2-benzylcarbamoyl-2-oxo-ethylcarbamoyl)-2-pyridin-2-yl-ethylcarbamoyl]-ethyl}-carbamic acid benzyl ester;
{(S)-1-[(S)-1-((S)-1-Benzyl-2-benzylcarbamoyl-2-oxo-ethylcarbamoyl)-2-(4-chloro-phenyl)-ethylcarbamoyl]-ethyl}-carbamic acid benzyl ester;
1,3-Dihydro-isoindole-2-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
2,5-Dimethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
2-Ethyl-2H-pyrazole-3-carboxylic acid {(S)-1-[(S)-1-((S)-1-benzyl-2-benzylcarbamoyl-2-oxo-ethyl carbamoyl)-2-phenyl-ethylcarbamoyl]-ethyl}-amide;
2-Methyl-5-trifluoromethyl-2H-pyrazole-3-carboxylic acid {(S)-1-[(S)-1-((S)-1-benzyl-2-benzylcarbamoyl-2-oxo-ethylcarbamoyl)-2-phenyl-ethylcarbamoyl]-ethyl}-amide;
£{(S)-1-[1-((S)-1-Benzyl-2-benzylcarbamoyl-2-oxo-ethylcarbamoyl)-2-(tetrahydro-pyran-4-yl)-ethylcarbamoyl]-ethyl}-carbamic acid benzyl ester;
{(S)-1-[1-((S)-1-Benzyl-2-benzylcarbamoyl-2-oxo-ethylcarbamoyl)-3,3,3-trifluoro-propylcarbamoyl]-ethyl}-carbamic acid benzyl ester;
{(S)-1-[(S)-1-((S)-1-Benzyl-2-benzylcarbamoyl-2-oxo-ethylcarbamoyl)-2-pyridin-4-yl-ethylcarbamoyl]-ethyl}-carbamic acid benzyl ester; or
Indan-2-carboxylic acid {(S)-1-[(S)-1-(2-benzylaminooxalyl-indan-2-ylcarbamoyl)-2-(4-methoxy-phenyl)-ethylcarbamoyl]-ethyl}-amide; or
pharmaceutically acceptable salts thereof.

17. The compound according to claim 1, wherein said compound is:
{(S)-1-[(S)-1-((S)-1-Benzyl-2-benzyl carbamoyl-2-oxo-ethylcarbamoyl)-2-(1H-indol-3-yl)-ethylcarbamoyl]-ethyl}-carbamic acid benzyl ester;
{(S)-1-[(S)-1-((S)-1-Benzyl-2-benzylcarbamoyl-2-oxo-ethylcarbamoyl)-2-(4-methoxy-phenyl)-ethylcarbamoyl]-ethyl}-carbamic acid benzyl ester;
Indan-2-carboxylic acid {(S)-1-[(S)-1-((S)-1-benzyl-2-benzylcarbamoyl-2-oxo-ethylcarbamoyl)-2-(1H-indol-3-yl)-ethylcarbamoyl]-ethyl}-amide; or
Indan-2-carboxylic acid {(S)-1-[(S)-1-((S)-1-benzyl-2-benzylcarbamoyl-2-oxo-ethylcarbamoyl)-2-(4-methoxy-phenyl)-ethylcarbamoyl]-ethyl}-amide; or
pharmaceutically acceptable salts thereof.

18. A pharmaceutical composition, comprising a therapeutically effective amount of a compound according to claim 1 and a pharmaceutically acceptable carrier.

19-22. (canceled)

23. A method for treating an inflammatory disease or disorder selected from rheumatoid arthritis, lupus and irritable bowel disease, comprising the step of administering a therapeutically effective amount of a compound according to claim 1 to a subject in need thereof.

24. (canceled)
